SEQUENCE LISTING (32 sequences)

SEQ ID NO: 1 - DNA, 2805 bp, Mycobacterium tuberculosis
ttggctgacccgaaaacgaagggccgtggcagcggcggcaatggcagcggccggcgactggtcatcgtcgagtcgcccaccaaggcgcgcaagctggcctcctacctgggctctggctacatcgtcgagtcctcccgggggcacatccgtgacttgccgcgggccgcgtcggatgtacccgcaaagtacaagtcgcagccgtgggcgcggctcggggtcaacgtcgacgccgacttcgaaccgctctacatcatcagcccggagaaacggagcaccgtcagcgagctcaggggcctgctcaaagacgtggacgagctgtatctggccacggatggggaccgtgagggcgaagctattgcctggcatctgctggaaaccctcaaaccgcgcataccggtaaagcggatggtcttccacgagatcaccgaaccggcgatccgcgccgccgccgagcacccccgcgacctagacatcgacctggtcgacgcgcaggagacccggcgcatcctggaccggctgtacggctacgaagtcagcccagtgctgtggaagaaggtcgcccccaagttgtcggcgggccgggtgcagtcggtggccacccgcatcatcgtggcgcgcgaacgcgaccgcatggcgttccgcagcgcggcctactgggacatccttgccaagctggatgccagcgtgtccgacccggacgccgcgccgcccaccttcagcgcccggctgacggccgtggctggccggcgggtggccactggccgcgatttcgactcgctgggcacgctgcgcaaaggcgacgaagtcattgtgctcgacgaggggagcgcgaccgcgttggccgcgggcctggatggcacgcagctgaccgtggcctcggccgaggagaagccctacgcccggcgcccgtacccgccgttcatgacctccacgctgcagcaagaggccagccgcaagctgcggttctccgccgagcggacgatgagcatcgcccagcggctgtacgaaaacggctacatcacctatatgcgtaccgactccaccacgctgtcggagtcggcgatcaacgccgcacgtacccaggcgcgccagctctacggcgacgagtacgtcgcgccggcgccgcgccaatacacccgcaaggtgaagaacgcccaggaagcgcacgaggctatccggcccgccggtgaaacgtttgccaccccggacgcggtgcgtcgcgaactcgacggtcccaacattgatgatttccggctctatgagctgatttggcaacgcaccgtagcctcgcagatggccgatgcgcggggcatgacgctgagcctgcggatcactggcatgtcggggcaccaggaggtggtgttctccgcgaccggacgcaccttgacgttcccgggcttcctcaaggcctacgtggagaccgtggacgagctggtcggcggcgaggctgacgatgccgagcggcgactgccccatctgaccccgggtcaacggttggacatcgtcgagttgaccccagacggccatgccaccaacccgccggcccgctacaccgaggcgtcgctggtcaaagcgctcgaggagctgggcatcggccgcccgtcgacctactcgtcgatcatcaagaccatccaggatcgcggctacgtgcacaagaagggcagtgcactggtgccgtcatgggtggcgttcgcggtaaccggtctgctcgagcagcatttcggtcggctcgtcgactacgacttcaccgcggcgatggaagacgagctcgacgagatcgccgccggcaacgagcgccgcaccaactggctcaacaacttctactttggtggcgatcacggtgtgcccgattcggtagcccgatcgggtggcctcaagaagcttgtcgggatcaatctcgagggcatcgacgcacgagaagtaaactctatcaagctttttgacgacacccacggacgccccatatatgttcgggtgggcaagaacggtccctacctggaacgtttggtggccggcgacaccggtgagcccacgccgcagcgggccaacctcagcgactcgattaccccggacgagctgactctacaggtggccgaagagctctttgccacaccgcaacagggacggactttgggcttggacccagaaaccggccacgagatcgtggccagggaaggccggtttgggccgtatgtgaccgagatcctgccggagcctgcggctgatgcggccgcggccgctcagggagtcaagaaacgccagaaggccgccgggcccaaaccgcgcaccggttcgttgctgcggagcatggacctacagacggtcaccctcgaagacgcgctgaggctgctgtcactgccgcgcgtggtcggagtggaccccgcctcgggtgaggagatcaccgcgcagaacgggcgctacggaccgtatctaaagcgcggcaacgattctcgatcactggtcaccgaagaccagatattcaccatcacgctcgacgaagccctgaagatctacgcagagccgaaacgtcgtggccggcaaagcgcttcggctccgccgctgcgcgagctgggaacagatccggcgtcgggcaagccaatggtcatcaaggacggccgattcgggccgtacgtcaccgacggtgagaccaatgccagcctgcgtaagggcgacgacgtggcttccataaccgacgagcgcgccgccgagctgttggccgatcgccgagcccggggtccggcaaaacggccagccaggaaagctgcccggaaggtgccggcgaagaaggcagccaagcgcgactag

SEQ ID NO: 2 - protein, 934 aa, Mycobacterium tuberculosis
MADPKTKGRGSGGNGSGRRLVIVESPTKARKLASYLGSGYIVESSRGHIRDLPRAASDVPAKYKSQPWARLGVNVDADFEPLYIISPEKRSTVSELRGLLKDVDELYLATDGDREGEAIAWHLLETLKPRIPVKRMVFHEITEPAIRAAAEHPRDLDIDLVDAQETRRILDRLYGYEVSPVLWKKVAPKLSAGRVQSVATRIIVARERDRMAFRSAAYWDILAKLDASVSDPDAAPPTFSARLTAVAGRRVATGRDFDSLGTLRKGDEVIVLDEGSATALAAGLDGTQLTVASAEEKPYARRPYPPFMTSTLQQEASRKLRFSAERTMSIAQRLYENGYITYMRTDSTTLSESAINAARTQARQLYGDEYVAPAPRQYTRKVKNAQEAHEAIRPAGETFATPDAVRRELDGPNIDDFRLYELIWQRTVASQMADARGMTLSLRITGMSGHQEVVFSATGRTLTFPGFLKAYVETVDELVGGEADDAERRLPHLTPGQRLDIVELTPDGHATNPPARYTEASLVKALEELGIGRPSTYSSIIKTIQDRGYVHKKGSALVPSWVAFAVTGLLEQHFGRLVDYDFTAAMEDELDEIAAGNERRTNWLNNFYFGGDHGVPDSVARSGGLKKLVGINLEGIDAREVNSIKLFDDTHGRPIYVRVGKNGPYLERLVAGDTGEPTPQRANLSDSITPDELTLQVAEELFATPQQGRTLGLDPETGHEIVAREGRFGPYVTEILPEPAADAAAAAQGVKKRQKAAGPKPRTGSLLRSMDLQTVTLEDALRLLSLPRVVGVDPASGEEITAQNGRYGPYLKRGNDSRSLVTEDQIFTITLDEALKIYAEPKRRGRQSASAPPLRELGTDPASGKPMVIKDGRFGPYVTDGETNASLRKGDDVASITDERAAELLADRRARGPAKRPARKAARKVPAKKAAKRD

SEQ ID NO: 3 - DNA, 2520 bp, Plasmodium falciparum
atgcaatcaatggaaataaatgataataacagtatcaagaatgaaagtacatctgatgatgatatattaattaataaaattaaacaaaacttgggtaataataaatcatgtaattctagatcttccaaaaaggaatctataaaaaagcaaaagagcaattctgaacttggtataaaaaagaacacaaagaaatcattaggtataaaaaaagaggaagaaaaaaaaaaacaaataagcaaaagaaaaagtaatgaactaaaagaaaaaaataatttgaaagagggaaaaaagaaatatgtggaaaaaaaatctagaacagtaaaagatgaaaccaagttaacgaatgttataaaaaaagaaactcaaaataataagaaacctaaaaaattacttaaaaaatcagaagaaaattttgaaccaataaatagatggtgggaaaaaatagatgatcaaacagatatacaatggaattatttagaacatcgaggattaatattttcccctccatacgttcaacatcatgtaccaattttttataaaagtataaaaattgaattaaatgcaaaatcagaagaattagctacctattggtgtagtgcaattggtagtgattattgtacaaaagaaaagtttatattaaatttttttaaaacatttataaatagtttagaaaatgataatattataaaacaagagaatgaaacgaaattaaaaaaaggagatatatctaattttaagtttattgattttatgccaatcaaagatcatttattaaaattaagagaagaaaagttaaataaaacaaaagaagaaaaagaagaggaaaaaaaaatgagaatggaaaaagaattaccatatacatatgcgttagttgattggattcgtgaaaagatatcaagtaataaagcagaaccacctgggttatttagaggaagaggagaacatccaaaacaaggtttattaaaaaaaagaatttttccagaagatgttgtaattaatattagtaaagatgcacctgtaccacgattatatgataatatgtgtggacataattggggtgatatatatcatgataataaagtaacatggttagcttattataaagatagtataaatgatcaaataaaatatacttttttatctgctcaatcaaaatttaaaggatataaagatcttatgaaatatgaaaatgctcgaaaattaaaatcatgtgttcataaaattagggaagattataaaaataaaatgaaaaataaaaatattattgataaacaattaggaacagctgtttatttaatagattttctagcattaagagtaggaggagaaaaagatatcgatgaagaagcagatactgtaggttgttgtagtttaagagtagaacatattagttttgcacacgatataccttttaaaagtgtagattcaaaagaacaaaaaacaaatgatgaaaaagtaaataaaataccattaccaacaaatttagaaagtatttcatcagaagattgttatataactttagattttttaggaaaagatagtatacgatattttaatacagtcaaaatagataaacaagcatatattaatataataatattttgtaaaaataaaaatagagatgaaggagtttttgatcaaataacttgttcaaaattaaatgaatatctaaaagaaattatgcctactttatcagctaaagtgtttcgtacatataatgcttcaattacattagatcaacaattaaaaagaataaaagaagtttatggaaaaacaacatattcattatattctggtgaaacagaattacacaaatcgaaaaaaagaaaatctagccatttaacttcagatacaaatatattaagtgatgcaagtgattctactattaatgatgtaaataacgagtatgatgaaaatggaataaataaaaaactatcatatgctactactgtaggaaaagaaaatgatgtcgatgataaaaactcaccaatagaagttgacgtttcaaatataaatgaacttattaatttttacaataatgcaaatagagaagtagccatattatgtaaccatcaaagaagtattccaaaacaacatgatacaactatgtcaaaaataaaaaaacaaattgaattatataatgaagatataaaagaatataaaaaatatttgcaacatttaaaaaaaaatagtgataaaaaatttatctttgtttcgaaagtttctactttagatggaactttaagaccaaataaagtcaaagaaaatatgaaagaagaatcttgtaaaaaaaaactaattactcttataaaaaaagttgaattattaaataaccaaatgaaagtaagagatgataataaaactattgctttaggtacatctaaaattaattatatggatccaagaataactgttgctttttgtaaaaaatttgaaatacccatagaaaaagtatttaatagaagtttaagacttaaatttccttgggccatgtttgctacaaaaaattttacattttaa

SEQ ID NO: 4 - protein, 839 aa, Plasmodium falciparum
MQSMEINDNNSIKNESTSDDDILINKIKQNLGNNKSCNSRSSKKESIKKQKSNSELGIKKNTKKSLGIKKEEEKKKQISKRKSNELKEKNNLKEGKKKYVEKKSRTVKDETKLTNVIKKETQNNKKPKKLLKKSEENFEPINRWWEKIDDQTDIQWNYLEHRGLIFSPPYVQHHVPIFYKSIKIELNAKSEELATYWCSAIGSDYCTKEKFILNFFKTFINSLENDNIIKQENETKLKKGDISNFKFIDFMPIKDHLLKLREEKLNKTKEEKEEEKKMRMEKELPYTYALVDWIREKISSNKAEPPGLFRGRGEHPKQGLLKKRIFPEDVVINISKDAPVPRLYDNMCGHNWGDIYHDNKVTWLAYYKDSINDQIKYTFLSAQSKFKGYKDLMKYENARKLKSCVHKIREDYKNKMKNKNIIDKQLGTAVYLIDFLALRVGGEKDIDEEADTVGCCSLRVEHISFAHDIPFKSVDSKEQKTNDEKVNKIPLPTNLESISSEDCYITLDFLGKDSIRYFNTVKIDKQAYINIIIFCKNKNRDEGVFDQITCSKLNEYLKEIMPTLSAKVFRTYNASITLDQQLKRIKEVYGKTTYSLYSGETELHKSKKRKSSHLTSDTNILSDASDSTINDVNNEYDENGINKKLSYATTVGKENDVDDKNSPIEVDVSNINELINFYNNANREVAILCNHQRSIPKQHDTTMSKIKKQIELYNEDIKEYKKYLQHLKKNSDKKFIFVSKVSTLDGTLRPNKVKENMKEESCKKKLITLIKKVELLNNQMKVRDDNKTIALGTSKINYMDPRITVAFCKKFEIPIEKVFNRSLRLKFPWAMFATKNFTF

SEQ ID NO: 5 - DNA, 67 bp, Artificial Sequence
  Oligonucletide primer or probe
cagagtgcgcagttggcctcaatgcacatgtttggctccgagcgagcttccgcttgacatcccaata

SEQ ID NO: 6 - DNA, 77 bp, Artificial Sequence
  Oligonucleotide primer or probe
cagagtgcgcagttggtctctcctcaatgcacatgtttggctcctctctgagcgagcttccgcttgacatcccaata

SEQ ID NO: 7 - DNA, 10 bp, Artificial Sequence
  Oligonucletide primer or probe
nncgcttgnn

SEQ ID NO: 8 - DNA, 108 bp, Artificial Sequence
  Oligonucletide primer or probe
tctagaaagtataggaacttcgaacgactcagaatgactgtgaagatcgcttatcctcaatgcacatgtttggctcccattctgagtcgttcgaagttcctatacttt

SEQ ID NO: 9 - DNA, 108 bp, Artificial Sequence
  Oligonucletide primer or probe
catacattatacgaagttatgagcgtctgagtatgactgtgaagatcgcttatcagtgaatgcgagtccgtctactcatactcagacgctcataacttcgtataatgt

SEQ ID NO: 10 - DNA, 96 bp, Artificial Sequence
  Oligonucletide primer or probe
atttttaaactgtgaagatcgcttatttaaaaatttttctaagtcttttttcccctcaatgctgctgctgtactacgaaaaaagacttagaaaaat

SEQ ID NO: 11 - DNA, 108 bp, Artificial Sequence
  Oligonucletide primer or probe
attataattttttggaacttcgaacgactcagaatgactgtgaagatcgcttatcctcaatgcacatgtttggctcccattctgagtcgttcgaagttccaaaaaatt

SEQ ID NO: 12 - DNA, 107 bp, Artificial Sequence
  Oligonucletide primer or probe
ttataattttttggaacttcgaacgactcagaatgactgtgaagatcgcttatcctcaatgcacatgtttggctcccattctgagtcgttcgaagttccaaaaaatt

SEQ ID NO: 13 - DNA, 108 bp, Artificial Sequence
  Oligonucletide primer or probe
tttataaagtataggaacttcgaacgactcagaatgactgtgaagatcgcttatcctcaatgcacatgtttggctcccattctgagtcgttcgaagttcctatacttt

SEQ ID NO: 14 - DNA, 104 bp, Artificial Sequence
  Oligonucletide primer or probe
aaattttttttggaacttcgaacgactcagaatgaggctcaatctaatggaccctcaatgcacatgtttggctcccattctgagtcgttcgaagttccaaaaaa

SEQ ID NO: 15 - DNA, 108 bp, Artificial Sequence
  Oligonucletide primer or probe
tttataaagtataggaacttcgaacgactcagaatgaggctcaatctaatggaccctcaatgcacatgtttggctcccattctgagtcgttcgaagttcctatacttt

SEQ ID NO: 16 - DNA, 108 bp, Artificial Sequence
  Oligonucletide primer or probe
tctagtaagtataggaacttcgaacgactcagaatgactgtgaagatcgcttatcctcaatgcacatgtttggctcccattctgagtcgttcgaagttcctatactta

SEQ ID NO: 17 - DNA, 110 bp, Artificial Sequence
  Oligonucletide primer or probe
atttttctaagtcttttagatcgaacgactcagaatgactgtgaagatcgcttatcctcaatgcacatgtttggctcccattctgagtcgttcgatctaaaagacttaga

SEQ ID NO: 18 - DNA, 14 bp, Artificial Sequence
  Oligonucletide primer or probe
tctagtaagnctta

SEQ ID NO: 19 - DNA, 14 bp, Artificial Sequence
  Oligonucletide primer or probe
atttttctantaga

SEQ ID NO: 20 - DNA, 23 bp, Artificial Sequence
  Oligonucletide primer or probe
cctcaatgcacatgtttggctcc

SEQ ID NO: 21 - DNA, 23 bp, Artificial Sequence
  Oligonucletide primer or probe
cagtgaatgcgagtccgtctact

SEQ ID NO: 22 - DNA, 23 bp, Artificial Sequence
  Oligonucletide primer or probe
cctcaatgctgctgctgtactac

SEQ ID NO: 23 - DNA, 18 bp, Artificial Sequence
  Oligonucletide primer or probe
actgtgaagatcgcttat

SEQ ID NO: 24 - DNA, 18 bp, Artificial Sequence
  Oligonucletide primer or probe
aggctcaatctaatggac

SEQ ID NO: 25 - DNA, 108 bp, Artificial Sequence
  Oligonucletide primer or probe
agaaaaatttttaaaaaaactgtgaagatcgcttatttttttaaaaatttttctaagtcttttagatccctcaatgctgctgctgtactacgatctaaaagacttaga

SEQ ID NO: 26 - DNA, 108 bp, Artificial Sequence
  Oligonucletide primer or probe
tctagaaagtataggaacttcgaacgactcagaatgaggctcaatctaatggaccctcaatgcacatgtttggctcccattctgagtcgttcgaagttcctatacttt

SEQ ID NO: 27 - DNA, 34 bp, Artificial Sequence
  Oligonucletide primer or probe
ccaaccaaccaaccaaataagcgatcttcacagt

SEQ ID NO: 28 - DNA, 34 bp, Artificial Sequence
  Oligonucletide primer or probe
ccaaccaaccaaccaagtccattagattgagcct

SEQ ID NO: 29 - DNA, 34 bp, Artificial Sequence
  Oligonucletide primer or probe
ccaaccaaccaaccaacatagagtcctggtgagc

SEQ ID NO: 30 - DNA, 23 bp, Artificial Sequence
  Oligonucletide primer or probe
gtagtacagcagcagcattgagg

SEQ ID NO: 31 - DNA, 23 bp, Artificial Sequence
  Oligonucletide primer or probe
ggagccaaacatgtgcattgagg

SEQ ID NO: 32 - DNA, 20 bp, Artificial Sequence
  oligonucletide primer or probe
agacggactcgcattcactg

(19) United States
(12) Patent Application Publication
(10) Pub. No.: US 2015/0018228 A1
(43) Pub. Date: Jan. 15, 2015

(54) ENZYME DETECTION BY MICROFLUIDICS

(71) Applicants: Jørn Erland Koch, Ry (DK); Magnus Stougaard, Hojbjerg (DK); Birgitta Ruth Knudsen, Viby J. (DK); Sissel Juul, Durham, NC (US); Kam Leong, Durham, NC (US); Megan Yi-Ping Ho, Durham, NC (US); Felicie F. Andersen, Aarhus C (DK)

(72) Inventors: Jørn Erland Koch, Ry (DK); Magnus Stougaard, Hojbjerg (DK); Birgitta Ruth Knudsen, Viby J. (DK); Sissel Juul, Durham, NC (US); Kam Leong, Durham, NC (US); Megan Yi-Ping Ho, Durham, NC (US); Felicie F. Andersen, Aarhus C (DK)

(21) Appl. No.: 14/241,442

(22) PCT Filed: Aug. 31, 2012
(86) PCT No.: PCT/DK2012/050327

§ 371 Date: Sep. 12, 2014

Related U.S. Application Data
(60) Provisional application No. 61/529,352, filed on Aug. 31, 2011.

(30) Foreign Application Priority Data
Aug. 31, 2011 (DK) PA 2011 70487

(51) Int. Cl. C12Q 1/68

(52) U.S. Cl. CPC C12Q 1/6893; C12Q 1/689
USPC 506/9; 435/6.15; 435/6.12; 435/6.11

(57) ABSTRACT

Microfluidic-implemented methods of detecting an enzyme, in particular a DNA-modifying enzyme, are provided, as well as methods for detecting a cell, or a microorganism expressing said enzyme. The enzyme is detected by providing a nucleic acid substrate, which is specifically targeted by that enzyme.

### FIELD OF INVENTION

The present invention relates to methods of detecting enzymatic activities and microorganisms, as well as methods of diagnosing diseases caused by such microorganisms, wherein the detection and diagnostic methods are implemented in a microfluidic system.

### BACKGROUND OF INVENTION

By definition, enzymes convert numerous substrate molecules to products with changed molecular characteristics without being consumed by the process. Consequently, highly sensitive detection of biomolecules can be achieved by enzymatic signal enhancement. PCR amplification of specific nucleotide sequences presently provides by far the most sensitive mean for detecting important biomarkers. However, for some purposes e.g. diagnosis of pathogen infections, where time of detection is a major concern, a serious drawback of PCR is the need for thermal cycling, hampering quantification and necessitating sophisticated equipment unavailable to many physicians.

One powerful isothermal amplification technique is the so-called Rolling-Circle-Amplification (RCA), by which a circular DNA template is converted to a ˜10³ tandem repeat product (RCP), visualizable at the single-molecule level upon hybridization of fluorescent probes. In combination with RCA, circular or circularizable nucleotide biosensors have successfully been employed to detect specific nucleic-acid sequences, proteins, or small-molecules (Lizardi, P. M. et al. Mutation detection and single-molecule counting using isothermal rolling-circle amplification. Nat Genet. 19, 225-232 (1998); Nallur, G. et al. Signal amplification by rolling circle amplification on DNA microarrays. Nucleic Acids Res 29, E118 (2001); Smolina, I., Miller, N. S. & Frank-Kamenetskii, M. D. PNA-based microbial pathogen identification and resistance marker detection: An accurate, isothermal rapid assay based on genome-specific features. Artif DNA PNA XNA 1, 76-82 (2010); Larsson, C. et al. In situ genotyping individual DNA molecules by target-primed rolling-circle amplification of padlock probes. Nat Methods 1, 227-232 (2004); Konry, T., Smolina, I., Yarmush, J. M., Irimia, D. & Yarmush, M. L. Ultrasensitive detection of low-abundance surface-marker protein using isothermal rolling circle amplification in a microfluidic nanoliter platform. Small 7, 395-400 (2011); Yang, L., Fung, C. W., Cho, E. J. & Ellington, A. D. Real-time rolling circle amplification for protein detection. Anal Chem 79, 3320-3329 (2007); Cho, E. J., Yang, L., Levy, M. & Ellington, A. D. Using a deoxyribozyme ligase and rolling circle amplification to detect a non-nucleic acid analyte, ATP. J Am Chem Soc 127, 2022-2023 (2005)). For diagnostic purposes, however, the disadvantage of most methods of the prior art is the need of extensive sample preparation and/or washing procedures and the formation of only one-to-a-few RCPs per target molecule.

An example of a parasitic disease for which there is a need for efficient low cost and reliable diagnostic tools is malaria, which according to WHO, has a prevalence of 300-500 million cases worldwide. Another example is tuberculosis, where approximately 30% of the human population is expected to be infected with tuberculosis. While malaria primarily affects the poorest regions of the world, tuberculosis (TB) is more widespread in both developing and developed countries. Tuberculosis is a global disease, which pose such a big problem that the World Health Organization in 1993 ruled disaster alarm. The majority of tuberculosis sufferers are found in the third world countries, in particular Africa and Southeast Asia, but there are also cases of tuberculosis in western countries, both among natives and immigrants. The global mobility of tuberculosis is increased due to global traffic and tourism, and the problems of the global prevalence of tuberculosis is underscored by the high prevalence of multidrug-resistant tuberculosis that can not be treated with traditional medicine, in particular the Baltic countries.

Tuberculosis is an infectious disease caused by inhalation of tuberculosis bacteria (Mycobacteria tuberculosis). These bacteria attack primarily the lungs, and cause a slight infection during the first six weeks without any serious symptoms. From the lungs, the bacteria can spread through the bloodstream to other organs, although still without necessarily doing any damage at first. In many cases, the infection is fought, if the infected person has a good immune system, however, months or years later, the disease may break out in both lungs and other organs if the immune system is weakened for various reasons. Today, outbreak of tuberculosis often occurs in connection with immune system weakening associated with HIV infection, in particular on the African continent. If tuberculosis is spread further in western countries, tuberculosis outbreak are likely to occur also among cancer patients and other patients, where the immune system is challenged.

A person with active tuberculosis infects on average 10 to 15 other people. Infection occurs through the air with tuberculosis bacteria in saliva droplets from cough or sputum from the patient being inhaled by others. Symptoms of tuberculosis such as heavy coughing and spitting does at least in the initial phases of the disease appear very alarming. The danger of infection is especially high in highly populated areas. The increasing global urbanization combined with increased migration is therefore an important factor in the rising number of tuberculosis cases worldwide.

Among the most important factors in fighting the spread of tuberculosis are effective and rapid methods of diagnosis so that persons with active and infectious tuberculosis can be isolated and subject to treatment. Already after fourteen days of antibiotic treatment, the risk of further transmission of the disease is prevented. To halt the spread of antibiotic resistant tuberculosis bacteria and to curb the spread of infection, WHO recommends a treatment strategy to reduced the DOTS (Directly Observed Treatment Short Course) which provides control and monitoring of patients and as such requires a safe, effective and rapid diagnosis of the disease. One of the problems when it comes to slow the spread of tuberculosis is that it has not yet succeeded in developing diagnosis methods that meet the necessary criteria, such as efficient at-bed-side diagnostic tools. Current diagnosis of TB relies on advanced instrumentation and facilities. Furthermore, diagnostics involve a several day long procedure. The method of the present invention, by contrast, is based on simple technology and can be performed and read-out at the bed-side within a few hours, provided that suitable platform development is achieved. When considering that each untreated TB patient on average transfers the infection to 10-15 other persons, early diagnosis allowing early treatment, which immediately prevents transfer of the disease, is of utmost importance.

### SUMMARY OF INVENTION

The present invention broadly relates to microfluidics-implemented methods of detecting enzymes, and microorganisms associated with said enzymes.

In one aspect, the present invention relates to a method of detecting an enzyme, preferably a DNA-modifying enzyme or an agent affecting the activity of such DNA-modifying enzymes, in a sample, said method comprising

a) providing the sample
b) providing a nucleic acid substrate targeted by a said enzymes,
c) loading said sample of step a) and said nucleic acid substrate of step b) into a sample chamber comprising a flow through channel, wherein droplets comprising said sample and said nucleic acid substrate are generated,
d) transfer said droplets from said sample chamber to a droplet retaining means through said flow through channel,
e) capturing one or more single droplets in individual cavities of said droplet retaining means, wherein each single droplet is spatially isolated from other droplets, and
f) detecting, in one or more captured droplets, nucleic acid substrate processed by said enzyme, wherein the presence of processed nucleic acid substrate is indicative of the presence of said enzyme.

The detection of enzymatic activities by the method of the invention allows for the detection in a sample of a cell, cell type or microorganisms, which express said enzyme. Thus, the invention also in one aspect relates to a method of identifying a microorganism expressing a specific enzyme in a sample, said method comprising

a) providing the sample
b) providing a nucleic acid substrate targeted by said specific enzyme of said microorganism,
c) loading said sample of step a) and said nucleic acid substrate of step b) into a sample chamber comprising a flow through channel, wherein droplets comprising said sample and said nucleic acid substrate are generated,
d) transfer said droplets from said sample chamber to a droplet retaining means through said flow through channel,
e) capturing one or more single droplets in individual cavities of said droplet retaining means, wherein each single droplet is spatially isolated from other droplets, and
f) detecting, in one or more captured droplets, nucleic acid substrate processed by said specific enzyme of said cell, cell type or microorganism, wherein the presence of processed nucleic acid substrate is indicative of the presence of said microorganism.

The enzyme detected in the above methods is preferably a DNA-modifying enzyme or an enzyme, protein or agent affecting a DNA modifying enzyme. For example, the enzyme is selected from the group consisting of nucleases, ligases, recombinases, topoisomerases and helicases, preferably a type I topoisomerase.

The invention also in a more specific aspect relates to a method of identifying a type I topoisomerase-expressing microorganism in a sample by a detection assay, which is implemented in a microfluidic system. The detection assay is based on the identification of a type I topoisomerase catalytic activity in the sample by providing a substrate which is specifically targeted and processed by a type I topoisomerase of said microorganism.

Thus, in one aspect, the present invention relates to a method of identifying a type I topoisomerase-expressing cell, cell type or microorganism in a sample, said method comprising

a) providing the sample
b) providing a nucleic acid substrate targeted by a type I topoisomerase of said microorganism,
c) loading said sample of step a) and said nucleic acid substrate of step b) into a sample chamber comprising a flow through channel, wherein droplets comprising said sample and said nucleic acid substrate are generated,
d) transfer said droplets from said sample chamber to a droplet retaining means through said flow through channel,
e) capturing one or more single droplets in individual cavities of said droplet retaining means, wherein each single droplet is spatially isolated from other droplets, and
f) detecting, in one or more captured droplets, nucleic acid substrate processed by said type I topoisomerase of said cell, cell type or microorganism, wherein the presence of processed nucleic acid substrate is indicative of the presence of said cell, cell type or microorganism.

Since, a type I topoisomerase-expressing microorganism identified by the method defined above may be involved in disease or pollution, the present also pertains to methods of determining a disease associated with a type I topoisomerase-expressing microorganism and/or contamination of e.g. foods or water with such microorganisms. So, the present invention also relates to methods for diagnosis, treatment, amelioration and/or prevention of diseases, which are associated with a microorganism, for example infectious diseases, in particular malaria and tuberculosis. The invention also relates to methods for detection of microorganisms associated with infectious or parasitic diseases, in particular, Plasmodium and Mycobacterium.

Thus, in one aspect, the present invention relates to a method of determining a disease in a subject, said method comprising identifying a cell, cell type or microorganism in a sample from said subject by a method comprising the steps of

a) providing the sample
b) providing a nucleic acid substrate targeted by an enzyme, such as a type I topoisomerase of said cell, cell type or microorganism,
c) loading said sample of step a) and said nucleic acid substrate of step b) into a sample chamber comprising a flow through channel, wherein droplets comprising said sample and said nucleic acid substrate are generated,
d) transfer said droplets from said sample chamber to a droplet retaining means through said flow through channel,
e) capturing one or more single droplets in individual cavities of said droplet retaining means, wherein each single droplet is spatially isolated from other droplets, and
f) detecting, in one or more captured droplets, nucleic acid substrate processed by said enzyme, such as type I topoisomerase of said cell, cell type or microorganism, wherein the presence of processed nucleic acid substrate is indicative of the presence of said cell, cell type or microorganism,
wherein the presence of said microorganism in said sample is indicative of said disease. In preferred embodiments, the disease is an infectious disease, such as malaria and said microorganism is selected from the Plasmodium genus. In other preferred embodiments, the disease is an infectious disease, such as human and/or bovine tuberculosis and said microorganism is selected from the Mycobacterium genus, for example Mycobacterium tuberculosis for humans and Mycobacterium bovis for bovines.

In yet another aspect, the invention relates to a method for evaluating the effect of an agent on a cell, cell type or microorganism in a sample, said method comprising

a) providing a sample comprising said enzyme, cell, cell type and/or microorganism,
b) providing a nucleic acid substrate targeted by said enzyme, and/or an enzyme of said cell, cell type and/or microorganism,
c) providing a chemical agent,
d) loading said sample of step a), said nucleic acid substrate of step b) and said agent of step c) into a sample chamber comprising a flow through channel, wherein droplets comprising said sample, nucleic acid substrate and agent are generated,
e) transfer said droplets from said sample chamber to a droplet retaining means through said flow through channel,
f) capturing one or more single droplets in individual cavities of said droplet retaining means, wherein each single droplet is spatially isolated from other droplets, and
g) detecting, in one or more captured droplets, nucleic acid substrate processed by said enzyme, and/or enzyme of said cell, cell type and/or microorganism, wherein a chemical agent capable of reducing the amount of processed nucleic acid substrate has an inhibitory effect on said enzyme, and/or enzyme of said cell, cell type and/or microorganism.

The enzyme is preferably a DNA-modifying enzyme, such as most preferably a type I topoisomerase.

### DESCRIPTION OF DRAWINGS

FIG. 1. Design and test of pfTopI specific substrate. A. shows the pfTopI cleavage sites on a selected doublestranded DNA fragment containing the classical hexadeceameric sequence from tetrahymena rDNA, which is a well know preferred cleavage site for nuclear type IB topoisomerases. B. Shows the five substrates tested for circularization by pfTopI and hTopI. C. A schematic illustration of the RCA based detection of pfTopI cleavage-ligation activity exemplified by Su2. Right panel shows how cleavage by pfTopI at the site indicated by arrow generate covalent cleavage intermediates, which supports ligation of the free 5′-OH end of the substrate resulting in the generation of closed circles. Right panel shows annealing of the pfTopI generated DNA circle to a specific primer attached to a glass surface. This primer supports Rolling Circle Amplification of the generated DNA circle (top left panel) generating 103 tandem repeats for a sequence complementary to the template DNA circle. The product of RCA is hybridized to specific fluorescent labelled probes (bottom left panel), allowing their visualization at the single molecule level using a fluorescent microscope. D. is an example of microscopic pictures obtained upon incubation of Su2 with either pfTopI (left panel) or hTopI (right panel) followed by RCA and hybridization to fuorescent probes. The red dots (dark spots) represents single RCA products of circularized Su2. The green dots (light spots) represents single RCA products of a closed control circle added to the sample in a known concentration to allow quantification of the results. E. Graphic representation of the results obtained when incubating each of the substrates Su2-Su6 or Su1 in the presence of 400 or 500 mM NaCl (which prevented circularization by hTopI) with pfTopI using the RCA-based visualization approach. The amount of products generated by RCA of circularized substrate was quantified relative to the amount of products obtained by RCA of added spike-in control circle.

FIG. 2. A. a representative example of the view in the microscope obtained when whole cell extract from HEK293T without (left panel) or with (right panel) spike-in purified pfTopI was incubated with Su1 and Su2 prior to addition of known concentration of control circles, RCA and hybridization of the resulting products with fluorescent labelled probes. Blue spots represent products generated by RCA of control circles, green spots are products generated by RCA of Su1, and red spots are products are generated by RCA of Su2. B. a representative example of the view in the microscope obtaine when extracts from uninfected (left panel) or P. falciparum infected (right panel) RBC was incubated with Su1 and Su2 and analysed as described for “A”.

FIG. 3. A. a representative example of the view in the microscope obtained extracts from undiluted (left panel), two times diluted (middle panel) or five times diluted extracts from P. falciparum infected (right panel) RBC was incubated with Su1 and Su2 and analysed using the RCA-based detection system after addition of control circle. C. shows the results of subjecting a genomic DNA preparation obtained from uninfected (lanes 1 and 2) or P. falciparum infected RBCs for PCR analysis using P. falciparum specific (odd lane numbers) or Plasmodium sp. specific (even lane numbers) primers. In the samples loaded in lanes 3 and 4, 5 and 6, or 7 and 8 the genomic DNA preparation was diluted 105, 107 or 108 times before PCR analyses. The PCR products were separated in a 1% agarose gel and visualized by EtBr staining.

FIG. 4. Schematic representation of the biosensor setup. A. topoisomerase I substrate. B. Detection of topoisomerase I activity on a dumbbell substrate followed by rolling circle amplification detection.

FIG. 5. Comparison of human and Plasmodium falciparum type I topoisomerase activity at increasing salt concentrations. The signals on each pictures indicate single cleavage-ligation events mediated by type I topoisomerase detected in an RCA-based biosensor system using the substrate (Su1). pfTopI exhibits a considerably higher salt tolerance than does hTopI.

FIG. 6. Detection of human and Plasmodium falciparum type I topoisomerase cleavage-ligation events detected in an RCA-based biosensor system using the substrate (Su1) in an extract of HEK293T cells. Increasing the salt concentration enables the specific detection of pfTopI on a background of human cell content including hTopI in extracts from cell lines or human blood (S3 and S4).

FIG. 7. Detection of type I topoisomerase cleavage-ligation events, detected in an RCA-based biosensor system using the substrate (Su1), in uninfected and infected blood.

FIG. 8. Examples of the view in the microscope obtained when blood extract from infected (left panel) or noninfected (right panel) was incubated with Su1 and Su2 prior to addition of known concentration of control circles, RCA and hybridization of the resulting products with fluorescent labelled probes. Blue (dark) spots represent products generated by RCA of control circles, green (light) spots are products generated by RCA of Su1, and red spots (indicated by arrows) are products are generated by RCA of Su2.

FIG. 9. Alignment of human (H) and Plasmodium falciparum (P) type I topoisomerase.

FIG. 10. Detection of MtTopI is achieved by converting a MtTopI specific cleavage product to a closed circle, which is used as template for RCA.

FIG. 11. pfTopI substrates secondary structure

FIG. 12. Assay for detection of Mycobacterium tuberculosis TopI

FIG. 13. Overview of at-point-of-care rst line diagnosis suitable for low resource settings with no laboratory facilities and low-trained personnel (no electricity or other special facilities needed). Left panel: Adaptation of assay for reaction/readout device; Right panel: Schematic illustration of crude design for reaction/readout device

FIG. 14. Reaction steps for diagnosis of tuberculosis and/or detection of Mycobacterium tuberculosis. As reaction control a chip detecting human type I topoisomerase in the same clinical sample is used (based on the RCA principle)—a device with one inlet leading to two reaction chambers with directly coupled beads could be envisioned. The control chamber should be blank as a control for correct washing of the device. All reactions can be performed within 20-40 degree Celsius. The device can be operated by minimally trained personnel and requires no electricity. Readout is performed by the naked eye. The device and similar devices may be operated by low-trained personnel and are also suitable for self-testing.

FIG. 15. The combined REEAD-microfluidic experimental setup

(a) S(TopI) and S(Flp) are each composed of an oligonucleotide that folds onto itself to allow cleavage-ligation by hTopI and Flp, respectively. These reactions circularize the substrates. S(TopI), S(Flp), and S(control) all contain a specific primer annealing p-element and a probe annealing i-element. The circles allow solid-support RCA generating ˜103 tandem repeat RCPs that are visualized in a microscope at the single-molecule level by hybridization of fluorescent probes. (b) The microfluidic setup. Cells-to-be-analyzed, DNA substrate(s) and lysis buffer are, by competitions with oil, confined in picoliter droplets in which DNA circularization takes place. (c) The droplets are confined in a drop-trap on a primer-coated glass slide on which RCA takes place. (d) The result of measuring hTopI activity using five million cells/mL in the combined REEAD-microfluidic setup. As a positive control S(control) was applied together with S(TopI). hTopI and S(control) specific signals were visualized by FAM—(green/light spots) and Cy5—(blue/dark spots) labeled probes, respectively.

FIG. 16. Detection of enzyme activities in rare- or single cells.

(a) Five million cells/mL of HEK293 cells containing 2.5%, 0.25% or 0.25% Flp-recombinase expressing cells were analyzed for Flp-recombinase and hTopI activity using the REEAD-microfluidic setup. Drop-trap cavities containing red signals (dark spots) corresponding to Flp-recombinase activity were selected. (b) Shows the percentage of red signals (dark spots) in five cavities of the drop-trap when five million cells/mL containing 2.5%, 0.25% or 0.25% Flp-recombinase expressing cells were analyzed for Flp-recombinase and hTopI activity (row 1-3) or when 0.5 million cells/mL containing 2.5% GFP-recombinase expressing cells were analyzed (row 4). (c) The result of analyzing the cell populations used in (a) for Flp-recombinase and hTopI activity in the “large-volume” bulk assay setup. (d) Same as (a) except that 0.5 million cells/mL containing 2.5% Flp-recombinase expressing cells was analyzed. hTopI and Flp-recombinase specific signals were visualized by FAM—(green/light) and TAMRA—(red/dark) labeled probes, respectively.

FIG. 17. Droplets in drop-trap. Light microscopy of drop-traps encapsulating 100 pL water-in-oil droplets. The drop-trap cavities are designed to each contain one droplet, which is spatially isolated from other droplets. Droplets are seen as round spheres in the cross-sections of the drop-trap grid.

FIG. 18. Theoretical estimate of the amount of cells in the picoliter droplets as a function of cell density. Encapsulation of cells within the 100 pl monodisperse droplets can be estimated as a Poisson (stochastic) distribution. According to this distribution, increasing the density of cells loaded into the system from 0.5 to five million cell/mL results in an increasing amount of cells encapsulated in each droplet. For example, when using the lowest cell density, 4.8% of droplets are expected to contain a single cell whereas only 0.1% of droplets are expected to contain two or more cells. This was also observed by Konry et al. 7, 11. Loading of five million cells/mL, on the other hand, will theoretically result in 30% of the droplets having single cells and 9.1% of droplets containing two or more cells.

FIG. 19. The density of cells loaded into the microfluidic device determines the number of cells per droplet. The middle of the image is a schematic illustration of the PDMS microfluidic device. As shown the device consists of three water phase inlets, an oil inlet, and an outlet for the generated droplets. Top panel, microscopic view of droplet entrapped cells resulting from loading HEK293 cells with a density of five million cells/mL into the microfluidic device. Consistent with the Poisson distribution (FIG. 18) this cell density results in approximately 40% of cell containing droplets. As evident these are not always single cells, and several cells are confined in the same droplet in approximately 9% of the cases. Bottom panel shows a microscopic view of the droplet encapsulated cells resulting form loading a cell concentration of one million cells/mL into the microfluidic device. Theoretically, loading of at this cell density ensures that no more than a single cell is confined in each droplet (FIG. 18). This was confirmed experimentally by observation of more than 5000 droplets revealing the encapsulation of one or no cells in each droplet. Note, that for the presented experiments, the substrate and lysis buffer, applied in channel two and three of the microfluidic device when performing REEAD experiments, were substituted by PBS to ensure the integrity of the cells since lysed cells cannot be detected in the light microscope used for visualization of cells and droplets in this experiment.

FIG. 20. Generation of Flp-recombinase expressing HEK293 cells. HEK293 cells were transfected with the plasmid, pCAG-Flpe:GFP, expressing recombinant Flpe fused to GFP. Flpe is a Flp-recombinase variant with enhanced thermostability and activity at 37° C., making it suitable for studies in mammalian cells 8. GFP (green fluorescent protein) was fused to Flpe to allow the number of Flpe expressing cells to be calculated by simply counting the number of green fluorescent cells. Note, that the fusion between GFP and Flpe does not affect the activity of the recombinase. Top and middle panels show a bright field image and a fluorescence image, respectively, of the transfected cells, while the bottom panel shows a merge of the bright field and fluorescence images. A transfection efficiency of 25% was determined by calculating the percentage of total cells expressing GFP.

FIG. 21. Development and test of nucleotide sensors for detection of pfTopI. a, schematic illustration of pfTopI cleavage sites on a double-stranded DNA fragment. Cleavage sites are indicated by an arrow denotated Cl1 or Cl2. Cleavage site Cl1 was shared between hTopI and pfTopI, while cleavage site Cl2 was specific for pfTopI. b, schematic illustration of nucleotide sensors (S1-S5) tested for reactivity with pfTopI. Each potential sensor folds into a hairpin structure. The single-stranded loop region contains an p-sequence matching a primer used to template RCA and a i-sequence allowing annealing of a specific fluorescent probe to generated RCPs. The double-stranded stems of S1-S5 contain different nucleotide sequences matching the degenerate consensus recognition sequence of nuclear type IB topoisomerases. c, schematic illustration of the REEAD setup exemplified by pfTopI reaction with S1. pfTopI mediated cleavage-ligation at the end of S1 generates a single-stranded DNA circle that is subjected to solid support RCA initiated from a glass slide-coupled primer with a sequence matching the p-sequence of S1. Unreacted S1 cannot template RCA. The generated RCPs are visualized microscopically upon hybridization of a fluorescent probe annealing to the i-region of RCPs. The putative cleavage site for pfTopI is indicated by an arrow. Grey ellipse labeled pfT denotes pfTopI while grey ellipse labeled pol denotes the Phi29 polymerase. d, shows an example of the microscopic view obtained upon incubation of S1 with pfTopI (top panel) or hTopI (bottom panel) in the REEAD setup. RCPs originating from circularized S1 and control circle were visualized by rhodamine—(red) and FITC—(green) labeled fluorescent probes, respectively. e, Quantitative depiction of the results obtained when incubating S1-S5 one at a time with purified pfTopI followed by RCA and microscopic visualization of RCA. The number of red and green fluorescent spots corresponding to individual RCPs originating from circularized S1-S5 and added control-circle, respectively, were counted in 15-30 microscopic views of each experiment. The bar chart shows the number of red spots divided by the number of green spots counted in three individual experiments.

FIG. 22. REEAD of pfTopI in crude biological samples.

a, Illustration of the nucleotide sensors used in the experiments shown in b, c, and e. b, microscopic view showing the result of REEAD analyses of nuclear extracts from HEK293T cells without (left panel) or with purified pfTopI spike-in (right panel) using the sensors shown in a. c, same as b except that extracts from blood from an uninfected (Sample #1, left panel) or pauci-parasitic malaria patient (Sample #2, right panel) were analyzed. d, shows a light-microscopic view of the microfluidic platform. Blood sample #2, nucleotide sensors and lysis buffer was loaded into three different channels in aqueous solution and by competition with oil confined in pL droplets in which the reaction took place. Mixing of droplet content was ensured by the serpentine channel of the device. e, is an example of a microscopic view obtained when analysing 200 pL of unprocessed sample #2 in the integrated REEAD-microfluidic channel setup. RCPs originating from circularized S1, S(TopI) and control circle were visualized by hybridization of rhodamine—(red), FITC—(green), Cy5—(blue) labeled fluorescent probes, respectively.

FIG. 23. Strategies to increase the sensitivity of pfTopI-specific REEAD.

a, Bar chart showing a quantitative depiction of the results obtained when analysing 2×-8× dilutions of extracts from the pauci-parasitic blood sample #2 by REEAD using only S1. To allow quantification, control-circle was added to the reaction mixtures before RCA. The efficiency of pfTopI-specific REEAD at these conditions was estimated by dividing the number of S1 specific signals with the number of control-circle specific signals in 15-30 microscopic views of three individual experiments. NC is a negative control in which sample #2 was replaced with extract from three different uninfected blood samples. No S1 originating signals were observed in 30 microscopic views of each of these reactions. b, Shows the result of spectrophotometric measurements obtained when analyzing 2×-128× dilutions of extracts from blood sample #2 in REEAD combined with HRP-mediated colorimetric readout in three individual experiments. PC is a positive control obtained by reacting S1 with purified recombinant pfTopI before HRP-REEAD and NC is a negative control obtained by incubation of S1 with extract from the uninfected blood sample #1 before REEAD analysis using HRP-mediated colorimetric readout.

FIG. 24. Comparison of DNA recognition by pfTopI and hTopI.

Recombinant hTopI and pfTopI were purified to homogeneity. The resulting protein fractions were analyzed in SDS-PAGE and visualized by Coomassie stain for purity and Western-blotting using a poly-clonal anti-TopI antibody for identity. The DNA recognition potentials of the two enzymes were compared by incubating each of them with 5″-end P32-labelled double-stranded DNA fragments (OL37/OL56 or OL62/OL63) as described in the Methods section below. To allow detection of cleavage, the anti-cancer drug camptothecin, which specifically inhibits the relegation step of TopI catalysis were added to the reaction mixtures while the religation reaction could be observed by omitting camptothecin from the reaction. The result of this analysis demonstrated that pfTopI recognizes and cleaves the sites cleaved by hTopI except that pfTopI unlike hTopI is also capable of cleaving double-stranded DNA a few bases upstream to a 3′-end followed by ligation of a protruding 5′-end.
a, left panel; shows the result of analysing purified pfTopI or hTopI by SDS-PAGE followed by coomassie stain. Lane 1, is a size marker with sizes of specific bands indicated to the left of the figure. Right panel, same as left panel except that the bands corresponding to pfTopI or hTopI were visualized by Western blotting using an poly-clonal anti-TopI antibody. b, top panel; is a schematic illustration of cleavage-ligation reactions shared by pfTopI and hTopI (an example of a cleavage site is indicated by an arrow marked Cl). Bottom panel, shows the result of incubating either pfTopI or hTopI with an end-labelled double-stranded DNA fragments in the absence or presence of camptothecin followed by denaturing gel-electrophoretic analysis of the results. The radioactive reaction products were visualized by PhosphorImaging. Bands representing the most pronounced cleavage products generated by both pfTopI and hTopI are indicated with Cl to the right of the gel picture. c, top panel, is a schematic illustration of the cleavage-ligation reaction mediated by pfTopI but not hTopI at the end of a double-stranded DNA fragment having a slightly protruding 5′-OH end. The pfTopI cleavage site is indicated by a arrow marked Cl. Bottom panel, shows the result of incubating pfTopI or hTopI in the absence or presence of camptothecin as indicated on the figure. A ligation product is only observed upon incubation of the substrate with pfTopI in the absence of camptothecin (lane 3). The mobility of this product correspond to 152 bases, which in turn correspond to pfTopI mediated cleavage 3 bases upstream to the 3′-end of the substrate followed by ligation of the protruding 5′-OH end of the non-cleaved strand. The cleavage product itself could not be observed directly (lane 4) due to a mobility very close to the substrate band. Note, that a trypsin-resistant peptide remains bound to the cleavage product causing a slight gel-electrophoretic retardation of this product. Hence, cleavage products arising from cleavage a few bases upstream to the 3′-end of the 75-mer are scattered by the substrate band. CPT, camptothecin; Cl, cleavage product; L, ligation product; S, substrate control; M, size marker. The sizes of marker bands are indicated to left of the gel-pictures.

FIG. 25. Circularization of S(TopI) by pfTopI. To investigate if pfTopI could react with and circularize the REEAD sensor S(TopI) previously demonstrated to react specifically with hTopI in human cell extract, purified recombinant pfTopI were incubated with S(TopI) and the result analyzed according to the REEAD protocol. As a positive control, control-circle was added to the reaction mixture before RCA.

The microscopic image shows the result of incubating purified pfTopI with S(TopI) followed by solid support RCA and visualization of resulting RCPs by hybridization to a rhodamine—(red) labeled probe. RCPs resulting from RCA of control-circles added to the reaction mixture were visualized by hybridization to a FITC—(green) labeled probe.

FIG. 26. The microfluidic lab-on-a-chip device. a, schematic illustration of the micro-fluidic channel device. In the microfluidic channel device, three merged aqueous streams containing blood cells, nucleotide sensors or low-salt lysis buffer are broken up by an oil stream to form a stable water-in-oil emulsion. The components confined in the aqueous picoliter droplets are lead through a serpentine channel to ensure adequate content mixing and reactions can subsequently take place within the droplets. Blood cells to be analyzed, lysis buffer and pfTopI(S1), S(TopI) and control-circle were fed to the system in three different channels (marked I, II, and III). By competition with oil (fed by channel IV) the three different components were confined in pL droplets, lead via a channel system to the outlet (V) and subsequently confined in the drop-trap device. The serpentine channel ensuring mixing of droplet content is indicated on the figure. b, shows the drop-trap device. Droplets were confined in cavities at the intersections in the drop-trap (right panel), and exsiccated onto a primer-coated glass slide (left and middle panel) to support RCA.

FIG. 27, The detection limit of REEAD combined with HRP colorimetric readout. The chart diagram shows the spectrophotometric readings obtained in three individual experiments measuring the activity of decreasing concentrations of purified recombinant pfTopI in REEAD using the HRP-mediated colorimetric readout.

### DETAILED DESCRIPTION OF THE INVENTION

A microfluidics-implemented nucleic acid based biosensor setup is provided herein. The system can be employed for detection of enzymes/enzymatic activities, particularly DNA-modifying enzymes, as well as for identifying specific cells, cell types or microorganisms, which express such specific enzymes. The microfluidics-implemented methods has potential use for at-point-of-care diagnosis of infectious disorders, such as malaria or tuberculosis as well as for the fast screening of drugs against the disease-causing Plasmodium or Mycobacterial pathogens. The system may also be used for sorting cells on the basis of their enzymatic expression profile, for example for sorting cells of a cancer tumour into separate population on the basis of their enzymatic activities for example the activity and specificity of type I topoisomerases of the different cells of the tumour. In the developed setup, specific detection of pathogenic microorganisms, such as malaria parasites, in biological samples, such as crude blood samples, is facilitated by specific enzymatic activities of the pathogenic microorganism, happening within nanometer dimensions, to micrometer-sized products readily detectable at the single molecule level in a fluorescence microscope. A specific example of such enzymatic activity is the conversion of single P. falciparum topoisomerase I (pfTopI) mediated cleavage-ligation events. The sensitivity of the presented microfluidics-implemented biosensor setup is clearly superior to standard cold immuno-based diagnostics.

The present invention relates to methods of detecting enzymatic activities and/or enzymes; identifying a microorganism, and methods of diagnosing infectious disorders caused by such microorganism. Furthermore, the invention relates to methods of treatment and compounds for use in the treatments of such infectious disorders. Moreover, the invention provides methods of sorting cells based on the enzymatic expression profile of the analysed cells.

The present invention, thus, provides a generic platform for detecting any enzyme or enzymatic activity, such as a DNA modifying enzyme or DNA modifying activity. The method of the invention is thus also applicable to the detection of any organism that expresses its own variant of such an enzyme, for example specific variant of a DNA modifying enzyme. The concept of the invention, however, extends to any enzyme system, such as nucleases, phosphatises, phosphorylases, topoisomerases and others, including DNA modifying enzymes systems, where a cascade of enzymes works to modify a nucleic acid target.

The method of the present invention is highly sensitive and simple, and requires only a short reaction time before an answer is obtained with respect to the presence of a microorganism.

### DEFINITIONS

To facilitate the understanding of the invention, some definitions of important terms are provided herein below.

As used herein, “nucleic acid” or “polynucleotide” or “oligonucleotide” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Polynucleotides can be composed of monomers that are naturally-occurring nucleotides (such as DNA and RNA), or analogs of naturally-occurring nucleotides (e.g., (alpha-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “polynucleotide” also includes so-called “peptide nucleic acids,” which comprise naturally-occurring or modified nucleic acid bases attached to a polyamide backbone. Nucleic acids can be either single stranded or double stranded.

The term “complement” or “complementary” in terms of a nucleic acid sequence refers to a polynucleotide having a complementary nucleotide sequence and reverse orientation as compared to a reference nucleotide sequence. For example, the sequence 5′ ATGCACGGG 3′ is complementary to 5′ CCCGTGCAT 3′.

“Complementary DNA (cDNA)” is a single-stranded DNA molecule that is formed from an mRNA template by the enzyme reverse transcriptase. Typically, a primer complementary to portions of mRNA is employed for the initiation of reverse transcription. Those skilled in the art also use the term “cDNA” to refer to a double-stranded DNA molecule consisting of such a single-stranded DNA molecule and its complementary DNA strand. The term “cDNA” also refers to a clone of a cDNA molecule synthesized from an RNA template.

The term ‘nucleotides’ as used herein refers to both natural nucleotides and non-natural nucleotides, which are capable of being incorporated into an oligonucleotide, such as a splice-switching oligonucleotide. Nucleotides may differ from natural nucleotides by having a different phosphate moiety, sugar moiety and/or base moiety. Nucleotides may accordingly be bound to their respective neighbour(s) in a template or a complementing template by a natural bond in the form of a phosphodiester bond, or in the form of a non-natural bond, such as e.g. a peptide bond as in the case of PNA (peptide nucleic acids).

The terms “disease” and “disorder” are used interchangeable herein, and are contemplated as synonymous. No specific meaning is intended from one of these terms over the other. A disease is understood as an abnormal condition of the organism that impairs bodily functions, and is associated with specific symptoms and signs. It may be caused by external factors, such as infectious and/or parasitic agents.

### Sequence Identity

The term “sequence identity” indicates a quantitative measure of the degree of homology between two amino acid sequences or between two nucleic acid sequences of equal length. If the two sequences to be compared are not of equal length, they must be aligned to give the best possible fit, allowing the insertion of gaps or, alternatively, truncation at the ends of the polypeptide sequences or nucleotide sequences. The sequence identity can be calculated, wherein Ndif is the total number of non-identical residues in the two sequences when aligned and wherein Nref is the number of residues in one of the sequences, preferably sequence identity is calculated over the full length reference as provided herein. Hence, the DNA sequence AGTCAGTC will have a sequence identity of 75% with the sequence AATCAATC (Ndif=2 and Nref=8). A gap is counted as non-identity of the specific residue(s), i.e. the DNA sequence AGTGTC will have a sequence identity of 75% with the DNA sequence AGTCAGTC (Ndif=2 and Nref=8).

With respect to all embodiments of the invention relating to nucleotide sequences, the percentage of sequence identity between one or more sequences may also be based on alignments using the clustalW software (http:/www.ebi.ac.uk/clustalW/index.html) with default settings. For nucleotide sequence alignments these settings are: Alignment=3Dfull, Gap Open 10.00, Gap Ext. 0.20, Gap separation Dist. 4, DNA weight matrix: identity (IUB).

The following terms are used to describe the sequence relationships between two or more polynucleotides: “predetermined sequence”, “comparison window”, “sequence identity”, “percentage of sequence identity”, and “substantial identity”.

A “predetermined sequence” is a defined sequence used as a basis for a sequence comparison; a predetermined sequence may be a subset of a larger sequence, for example, as a segment of a full-length DNA or gene sequence given in a sequence listing, such as a polynucleotide sequence as disclosed herein or may comprise a complete DNA or gene sequence. Generally, a predetermined sequence is at least 20 nucleotides in length, frequently at least 25 nucleotides in length, and often at least 50 nucleotides in length. Likewise, the predetermined sequence is that of the polypeptides of the invention.

The term “diagnosticum” refers in the present context to a compound or composition used in diagnosis of a disease or medical state. In the present text the diagnosticum is a binding member or a detection member of the present invention or active derivative thereof for use in the diagnosis of a disease or condition, as described herein above. The nucleic acid substrate of the invention may be used as a diagnosticum, Thus, in an aspect the invention relates to nucleic acid substrate according to the invention for use as a diagnosticum.

Cells, cell types and microorganisms. The present invention relates to methods for identification of specific cells, cell types and/or microorganisms. The term “cells” are meant to encompass cells of different origine, while the term “cell types” more refers to cells of the same origin, which may have undergone changes, which allows those cells to be distinguished. The methods of the invention are applicable for separating cells of different origin, such as parasitic cells from mammalian cells, but they are also applicable for distinguishing for example human cancer cells from human non-cancer cells. In the latter case, the cells are both human cells, but are different cell types, because one of the cell types has diverged into a cancerous cell, and the changes that the cell has undergone in the process of its transformation can be detected via altered enzymatic activities.

### Method for Enzyme Detection.

The present invention in one aspect relates to a microfluidics-implemented method of detecting an enzymatic activity in a sample. The invention relates to a method of detecting an enzyme in a sample, said method comprising

a) providing the sample
b) providing a nucleic acid substrate targeted by a said enzymes,
c) loading said sample of step a) and said nucleic acid substrate of step b) into a sample chamber comprising a flow through channel, wherein droplets comprising said sample and said nucleic acid substrate are generated,
d) transfer said droplets from said sample chamber to a droplet retaining means through said flow through channel,
e) capturing one or more single droplets in individual cavities of said droplet retaining means, wherein each single droplet is spatially isolated from other droplets, and
f) detecting, in one or more captured droplets, nucleic acid substrate processed by said enzyme, wherein the presence of processed nucleic acid substrate is indicative of the presence of said enzyme.

The enzyme is preferably a DNA-modifying enzyme, such as an enzyme selected from the group consisting of nucleases, ligases, recombinases, topoisomerases and helicases, preferably a type I topoisomerase. Details with respect to the method, such as nucleic acid substrates etc. are provided elsewhere herein.

### Method for Identification of Microorganism.

The detection of enzymatic activities by the method defined above also allows for the detection in a sample of specific cells or cell types, or microorganisms, which express said enzyme. Therefore, the present invention also relates to a method for the identification of an enzyme-expressing cell, cell type or microorganism in a sample, preferably a DNA-modifying enzyme, such as an enzyme selected from the group consisting of nucleases, ligases, recombinases, topoisomerases and helicases, preferably a type I topoisomerase. In a preferred embodiment, the cell, cell type or microorganism is a type I topoisomerase-expressing cell, cell type or microorganism.

Generally, the cell, cell type or microorganism is identified on the basis of its expression of a DNA modifying enzyme, which is specific for that particular cell type or microorganism, in particular DNA modifying enzymes which display a site-specific DNA modifying activity. In this way, specific nucleic acid substrates are employed, which comprise a sequence specifically targeted by the enzymes in question, where the processing of that substrate is indicative of the presence of that particular cell, cell type or microorganism. The cell, cell type or microorganism is identified on the basis of a detection of an enzymatic activity, such as type I topoisomerase activity, of that specific cell, cell type or microorganism. In humans and non-human mammals, plants, algae and so forth, the enzymatic activity, such as topoisomerase activity, of topoisomerases from exogenous microorganisms can be distinguished from the native enzymatic activity, such as topoisomerase activity, of that particular subject (humans and non-human mammals, plants, algae) based on the substrate used for detection of enzyme activity. The enzymes of the tested subject and the cell, cell type or microorganism can be distinguished by provision of substrates, for which an enzyme, such as type I topoisomerase, of the subject has a higher affinity for relative to the microorganism, and vice versa. The method of identifying a cell, cell type or microorganism of the invention comprises the following steps:

a) providing the sample
b) providing a nucleic acid substrate targeted by an enzyme, such as a type I topoisomerase of said cell, cell type or microorganism,
c) loading said sample of step a) and said nucleic acid substrate of step b) into a sample chamber comprising a flow through channel, wherein droplets comprising said sample and said nucleic acid substrate are generated,
d) transfer said droplets from said sample chamber to a droplet retaining means through said flow through channel,
e) capturing one or more single droplets in individual cavities of said droplet retaining means, wherein each single droplet is spatially isolated from other droplets, and
f) detecting, in one or more captured droplets, nucleic acid substrate processed by said enzyme, such as preferably a type I topoisomerase of said cell, cell type or microorganism, wherein the presence of processed nucleic acid substrate is indicative of the presence of said cell, cell type or microorganism.

The detection of processed nucleic acid substrate is then indicative of the presence of a cell, cell type or microorganism, which express a particular enzyme, such as topoisomerase, that targets the nucleic acid substrate provided in step b). Nucleic acid substrates, which are predominantly target by an enzyme, such as a type I topoisomerase, of one cell, cell type or microorganism, is then used for the detection of that specific microorganism. Examples of nucleic acid substrates, which are specifically targeted and processed by an enzyme, such as a type I topoisomerase, of a specific cell, cell type or microorganism are provided herein below.

As described herein below, the cell, cell type or microorganism is preferably a pathogenic cell, cell type or microorganism, and most preferably a microorganism involved in malaria or tuberculosis, such as Plasmodium falciparum or Mycobacterium tuberculosis, or Mycobacterium bovis. In a more specific application, the microorganism is identified in a sample from a human subject, and the nucleic acid substrate is then targeted predominantly by a type I topoisomerase of the microorganism and not or to a significantly lesser extent by human topoisomerase I. However, the cell or cell type may also be a cancer cell, which express a specific enzymatic activity, such as a specific topoisomerase I activity. In this case, the method of the present invention may be used for diagnosing a cancer, or staging a cancer on the basis of the expression of specific DNA-modifying enzymes, or by the relative activity of DNA-modifying enzymes. The method can be employed for analysing the relative or absolute level of cancer cells in a tumor, which express a certain enzyme or has a certain enzymatic activity.

The method of the present invention allows for detecting the quantitative presence of a cell, cell type or microorganism in the sample. Depending on the choice of detection method for detecting processed nucleic acid substrate, the enzymatic activity, such as topoisomerase activity, may be determined quantitatively. Quantitative detection methods such as rolling circle amplification allow such quantitative detection of enzymatic activity, such as topoisomerase activity, and thus also quantitative detection of the presence of cell, cell type or microorganisms.

### Microfluidic System

The present invention relates to methods of detecting an enzyme, such as a DNA-modifying enzyme; methods of identifying a specific cell, cell type or microorganism, such as type I topoisomerase-expressing cell, cell type or microorganism; methods of determining a disease associated with said enzyme, cell, microorganism or cell type; and methods for evaluating the effect of a chemical agent on the enzyme, cell, cell type or microorganism, as described elsewhere herein. A common feature of the methods of the invention is that they are implemented or at least partly implemented in a microfluidic setup.

The sample, which is subjected to analysis by any of the methods of the invention, is loaded into a sample chamber, which comprises at least one flow through channel. In particular, the sample chamber may comprise one or more inlet channels and/or one or more outlet channels. The sample chamber comprises at least one outlet channel, through which small droplets comprising the sample and nucleic acid substrate are transferred. The outlet channel may be formed as a serpentine channel, and serves for the components of the droplet to be adequately mixed. The enzymatic processing of the nucleic acid substrate by the enzyme, such as DNA-modifying enzyme, e.g. type I topoisomerase or recombinase, also preferably take place in the droplets, while travelling in the outlet channel. The microfluidic setup may also be adapted for multiplexing, in which case, the sample chamber comprise two or more outlet channel, where different nucleic acid substrates are loaded in each different outlet channel, thereby allowing several enzymatic activities, cells, cell types or microorganisms to be detected in parallel for the same sample.

continuous phase/carrier fluid/oil and disperse phase (aqueous reagents

The sample chamber also preferably comprises one or more inlet channels, for loading components into the microfluidic system. One inlet channel may direct the loading of a surfactant/carrier fluid/continuous phase, which surrounds the disperse phase/aqueous phase, which exists as droplets, which comprise sample and nucleic acid substrate. That fluid is preferably an oil, such as a fluorocarbon oil although other fluids are available for the same purpose. The sample and nucleic acid substrate thus leaves the sample chamber as water-in oil droplets, wherein the aqueous phase droplets are generated by competitions with the carrier fluid/continuous phase, such as oil, and confined in picoliter droplets in which the processing of the substrate, such as DNA circularization, takes place.

Sample, nucleic acid substrate, lysis buffer, and/or processing reaction buffer may be loaded into the sample chamber via one inlet channel or by individual inlet channels. For analysis of biological samples, a cell lysis buffer is preferably mixed with the sample, either prior to loading the sample in the sample chamber of the microfluidic device or loaded into the sample chamber independently of the sample via a designated inlet channel. In a preferred embodiment, the sample chamber of the microfluidic device comprises at least four inlet channels for the individual loading of sample, nucleic acid substrate, cell lysis buffer and oil, respectively.

The dimensions of the sample chamber and flow through channels are within order usually employed in the art. For example, in one embodiment, the one or more flow through channels, inlet channels and/or outlet channels have a diameter of less than 1000 micrometers, such as less than 500 micrometers, for example less than 400, such as less than 300, such as less than 500 micrometers, for example less than 400, such as less than 300, such as less than 200 micrometers, for example less than 100, such as less than 90, such as less than 80 micrometers, for example less than 70, such as less than 60, such as less than 50 micrometers, for example less than 40, such as less than 30, such as less than 25 micrometers, for example less than 20, such as less than 15, such as less than 10 micrometers, for example less than 5 micrometers. In one embodiment, the one or more flow through channels, inlet channels and/or outlet channels have a diameter of 10-50 micrometers, such as 15-45, for example 20-45, for example, 20-40, such as 20-35, such as 20-30, for example 20-25 or 25-30 micrometers, or approximately 25 micrometers in diameter.

The flow rate of the carrier fluid/surfactant and the disperse phase/aqueous phase reagents, such as sample/substrate/lysis buffer may be controlled independently for example by one or more syringe pumps. The independent flow of carrier fluid/surfactant/oil and aqueous reagents allows monodisperse water-in-oil droplets to be formed, for example at a frequency of 0.2-5 kHz, such as 0.3-4, such as 0.4-3, for example 0.5-2.5, such as 0.5-2 kHz, preferably at a frequency of 0.8-1.5 kHz. The droplet volume and generation frequency can be controlled by the flow rate ratio, determined by the competition between continuous phase/carrier fluid/oil and disperse phase (aqueous reagents: cells, lysis buffer and substrates. The continuous phase consisting of for example oil such fluorocarbon oil preferably load at a rate of 1-100 μl (microlitre)/min, such as 1-90, for example 1-80, such as 1-90, for example 1-80, such as 1-90, for example 1-80, such as 1-70, for example 1-60, such as 1-50, for example 10-50, such as 10-40, for example 10-30, such as 15-30, for example 15-25 such as 20-25, preferably about 22.5 μl (microlitre)/min. The disperse phase/aqueous reagents (such as sample, cells, lysis buffer and/or nucleic acid substrates preferably load at a rate which is significantly lower than the continuous (oil) phase. The disperse phase/aqueous reagents preferably load at a rate of 0.1-50 μl (microlitre)/min, such as 0.1-40, for example 0.1-30, such as 0.1-20, for example 0.1-15, such as 0.1-10, for example 0.5-10, such as 0.5-10, for example 1-10, such as 1-15, for example 1-10, such as 1-5, for example 1.5-5, such as 1.5-4, for example 2-3, preferably about 2.5

Thus, the size of the one or more of the flow through channels, in particular the outlet flow through channel, and the flow rate of the components applied via for example the inlet channels in particular the relative flow rate of the disperse phase/aqueous reagents comprising the sample/substrate/lysis buffer and the continuous phase/oil/fluid determine the size of the generated droplets. The size of the droplets is preferably within the picolitre range, such as between 10 and 1000 picolitres, such as 10-500, for example 10-400, for example 10-300, such as 10-200 for example 10-100 picolitres pr droplet. In one embodiment, the droplets has a volume of 500 pL or less, such as between 50 and 200 pL.

Each of the droplets preferably comprises only one cell. However, since the cells are loaded into the sample chamber as a solution of cells, some droplets may comprise more than one cell. Thus, in order to reach a minimum of droplets with more than one cell, the sample should be diluted to such an extent that the majority of droplets comprise 1 cell. Thus, on one embodiment, the sample comprise between approximately 500,000 and 10 million cells per mL, such as between approximately 1 million and 5 million cells per mL. The optimal cell concentration depends on the respective flow rates of the surfactant/continuous phase and the aqueous phase. In a preferred embodiment, the concentration of cells in the sample is adjusted, such that none of the generated droplets comprise 5 or more cells. In one embodiment, at least 90%, such as at least 91%, for example at least 92%, such as at least 93%, such as at least 94%, for example at least 95%, such as at least 96%, such as at least 97%, for example at least 98%, such as at least 99% of the droplets comprise 4 or less cells, such as 3 or less cells, for example 2 or less cells. In a preferred embodiment, at least 50%, such as at least 60%, for example at least 70%, such as at least 75%, for example at least 80%, such as at least 85%, for example at least 90%, such as at least 91%, for example at least 92%, such as at least 93%, such as at least 94%, for example at least 95%, such as at least 96%, such as at least 97%, for example at least 98%, such as at least 99% of the droplets comprise one or no cells. In a preferred embodiment, 60-99%, such as 70-99%, such as 70-95%, such as 75-95, for example 80-90 of the droplets comprise one or no cells. In one embodiment, between approximately 4 and 30% of the droplets comprises one cell, and approximately 0.1 to 10% of the droplets comprise two or more cells.

From the sample chamber, droplets are generated and transferred via an outlet flow through channel to a droplet retaining means, where one or more single droplets are captured in individual cavities and each single droplet is spatially isolated from other droplets

In the captured droplets, nucleic acid substrate is detected, which have been processed by the enzyme, which is analysed, for example DNA-modifying enzyme, preferably a type I topoisomerase. The presence of processed nucleic acid substrate is then indicative of the presence of said enzyme/enzymatic activity. The droplet retaining means is for example a solid support, which a number comprises individual cavities or pores for retaining individual droplets.

After being captured at the droplet retaining means, the droplets are preferably reduced in size by slight exsiccation. The presence of processed nucleic acid substrate in at least one individual droplet captured on the droplet retaining means can be detected by any suitable method. In a preferred embodiment, processed nucleic acid substrate is detected by rolling circle amplification as described herein below.

Processed nucleic acid substrate can be detected in each droplet, because the substrate is converted from a non-circular molecule, which does not support for example rolling circle amplification, for example a self-folding so-called dumbbell substrate, to a closed nucleic acid circle. That circle may then subsequently be subjected to Rolling Circle Amplification (RCA) leading to a Rolling Circle amplification Product (RCP) consisting of ˜10³tandem repeats of a sequence complementary to the DNA circles. Each RCP can be visualised at the single-molecule level in a fluorescence microscope by annealing to fluorescent-labelled probes giving rise to one fluorescent spot for each RCP. Since rolling circle amplification involves no thermal cycling, each RCP represents one closed DNA circle, which in turn represents a single cleavage-ligation event. In a preferred embodiment, the captured droplets are positioned on a glass slide, which is coated with DNA primer, which support amplification of processed, circularized nucleic acid substrate. For example, the means for retaining droplets (drop-trap) may be gently placed on top of a primer-coated glass slide.

The microfluidic setup allows for extremely sensitive and specific detection of enzymatic activities at the level of individual cells. Enzymes, such as type I topoisomerases can be detected at the aM level.

### Type I Topoisomerase

By definition enzymes convert substrate molecules to products with changed chemical or physical characteristics without being affected by the process. Hence, one enzyme can in general create indefinite amounts of product provided with sufficient substrates and, consequently, the most sensitive detection of pathogens imaginable relies on detection of species-specific enzymatic products.

According to the methods of the present invention, specific enzymes and/or enzymatic activities are detected on the basis of a detection of a nucleic acid substrate, which is specifically targeted and processed by that specific enzymes and/or enzymatic activities. Furthermore, according to the methods of the present invention, a cell, cell type or microorganism is identified in a sample by detecting a nucleic acid substrate which is targeted by a nucleic acid modifying enzyme system specific for said cell, cell type or microorganism. This detection method also forms the basis of the identification and diagnostic methods, compositions and uses of the present invention.

The methods of the invention extends to any enzyme system, such as nucleases, ligases, recombinases, phosphatases, phosphorylases topoisomerases and helicases, preferably type I topoisomerases. Further, nucleic acids modifying enzymes system, where a cascade of enzymes works to modify a nucleic acid target are also within the scope of the present invention.

In a preferred embodiment, the present invention relates to nucleic acid-based detection assays based on type I topoisomerase for the identification of a cell, cell type or microorganism via the detection of specific single enzymatic products mediated by topoisomerase I. In general, type I topoisomerases act by introducing single strand cuts in DNA followed by subsequent ligation of the generated nick in a reaction that involves the formation of a covalent enzyme-DNA cleavage intermediate.

So in a preferred embodiment of the methods and uses of the present invention, a cell, cell type or microorganism is identified in a sample by detecting a nucleic acid substrate which is targeted by a type I topoisomerase of said cell, cell type or microorganism. Type I topoisomerase targets double stranded nucleic acid molecules by binding a region of said nucleic acid molecule and cleaving a single strand of the duplex.

The cleavage reaction of type I topoisomerase can be conducted on a specific nucleic acid substrate, which upon cleavage is converted from a self-folding so-called dumbbell substrate to a closed nucleic acid circle. That circle may then subsequently be subjected to Rolling Circle Amplification (RCA) leading to a Rolling Circle amplification Product (RCP) consisting of ˜10³tandem repeats of a sequence complementary to the DNA circles. Each RCP can be visualised at the single-molecule level in a fluorescence microscope by annealing to fluorescent-labelled probes giving rise to one fluorescent spot for each RCP. Since rolling circle amplification involves no thermal cycling, each RCP represents one closed DNA circle, which in turn represents a single cleavage-ligation event. In one preferred embodiment, the captured droplets positioned on a glass slide, which is coated with DNA primer, which support amplification of processed, circularized nucleic acid substrate. For example, the means for retaining droplets (drop-trap) may be gently placed on top of a primer-coated glass slide.

False positives are avoided by depleting the reaction buffers for divalent cations, which is a prerequisite for the activity of most DNA modifying enzymes, including ligases, but not for type I topoisomerases such as pf-topoisomerase I and tuberculosis topoisomerase I. Thus in a preferred embodiment, the sample is depleted for divalent cations. Thus, an agent for depletion of divalent cations is added to the sample prior to its combination with nucleic acid substrate, or the substrate is mixed with such as agent for depletion of divalent cations in order to reduce the activity of other nuclease/topoisomerase enzymes.

Detection of type I topoisomerase activity is observed by identification of processed nucleic acid substrate. The nucleic acid substrate may be processed by either single strand cleavage and/or ligation. Thus, in one embodiment, the nucleic acid substrate is processed by cleavage, and in another embodiment, the substrate is processed by ligation by a type I topoisomerase of the relevant microorganism. In one embodiment, the substrate is processed by cleavage by the topoisomerase, and then ligated to another nucleic acid molecule or to it self to generate a circular molecule by an exogeneous, such as a recombinant, ligase. So, in one embodiment, ligation is catalyzed by said type I topoisomerase of said microorganism, by a heterogeneous ligase and/or by a recombinant ligase.

In a specific embodiment, the ligation is intramolecular ligation of the 3′-terminus of the nucleic acid substrate to the 5′-terminus of the nucleic acid substrate, thereby generating a circular nucleic acid product. Such a circular product is for example detectable by rolling circle amplification, as described elsewhere herein. In one embodiment, the substrate is processed by cleavage by said type I topoisomerase, followed by intramolecular ligation of the free 3′-terminus of the cleaved substrate to the 5′-terminus of the nucleic acid substrate, thereby generating a circular nucleic acid product.

### Nucleic Acid Substrate

The methods of the present invention employ nucleic acid substrates, which are targeted by the enzyme, such as a type I topoisomerase, which is detected according to the method of the invention, or which enzyme is specific for a cell, cell type or microorganism, the presence of which is to be determined by the method of the invention. The sequence and structure of the nucleic acid substrate is optimized with respect to the enzyme, such as specific topoisomerase activity of the respective cell, cell type or microorganism. Specific target sequences are targeted with higher efficiency by the enzyme, such as topoisomerases of certain cells, cell types or microorganism, or subjects than others, and in this way, the activity of an enzyme such as a type I topoisomerase of one cell, cell type or microorganisms, such as pathogenic and/or parasitic microorganisms can be distinguished from the enzymes, such as topoisomerases of other cells, cell types, microorganisms, or subjects, such as human and non-human mammal subjects.

So in the methods and kits of the present invention, the nucleic acid substrate is predominantly targeted by an enzymes, such as a type I topoisomerase of said cell o, cell type or microorganism and to a lesser extent by any enzyme, such as any type I topoisomerase native to said sample, or which is also located in the sample, for example which originates from another cell or cell type in the sample. The term “native” as used here, indicates that the enzyme, such as topoisomerase is the natural enzyme (topoisomerase), which is encoded by the cells of the sample, i.e. human cells if the sample originates from a human being, and bovine cells, if the sample originates from a bovine subject, and for example non-cancer cells, if the sample is a tumour sample. Thus, an enzymes, e.g. a type I topoisomerase, native to a human sample is a human enzyme, e.g. type I topoisomerase, and an enzyme, e.g. a type I topoisomerase native to a bovine sample is a bovine enzyme, e.g. bovine type I topoisomerase.

The nucleic acid substrate may be labelled, and/or hybridized to one or more nucleic acid probes, and detected via the respective label. The nucleic acid substrates may be coupled to a support. Such supports are well known to those of ordinary skill in the art and include, but are not limited to glass, plastic, metal, or latex. In particular aspects of the invention, the support can be planar or in the form of a bead or other geometric shapes or configurations known in the art.

In the methods of the invention, nucleic acid substrate is a double stranded nucleic acid molecule. The double stranded substrate is for example provided at two single molecules, which are hybridized, however, in a preferred embodiment, the double stranded substrate is provided as a single nucleic acid, which folds into a secondary hairpin structure comprising a double-stranded target region.

The nucleic acid substrate of the methods of the present invention is for example selected from any one of SEQ ID NO: 5-32. The nucleic acid substrate, in one embodiment, comprises a sequence selected from any one of SEQ ID NO: 5-32, a sequence at least 30%, 40%, 50%, 60%, 70%, 80%, such as at least 90%, for example at least 91%, 92%, 93%, 94%, 95%, 96%, 97%, 98%, such as at least 99% identical thereto, or a part of at least 5 consecutive nucleotides, such as at least 10, 15, 20, 30, 40, 50, 60, 70, 80, 90, such as at least 100 consecutive nucleotides, of any of said sequences.

In a specific embodiment, the method and/or kit comprises a nucleic acid substrate comprising a sequence selected from any one of SEQ ID NO: 8-19, a sequence at least 30%, 40%, 50%, 60%, 70%, 80%, such as at least 90%, for example at least 91%, 92%, 93%, 94%, 95%, 96%, 97%, 98%, such as at least 99% identical thereto, or a part of at least 5 consecutive nucleotides, such as at least 10, 15, 20, 30, 40, 50, 60, 70, 80, 90, such as at least 100 consecutive nucleotides, of any of said sequences. In this case, the microorganism is selected from the Plasmodium Genus, for example the microorganism is Plasmodium falciparum.

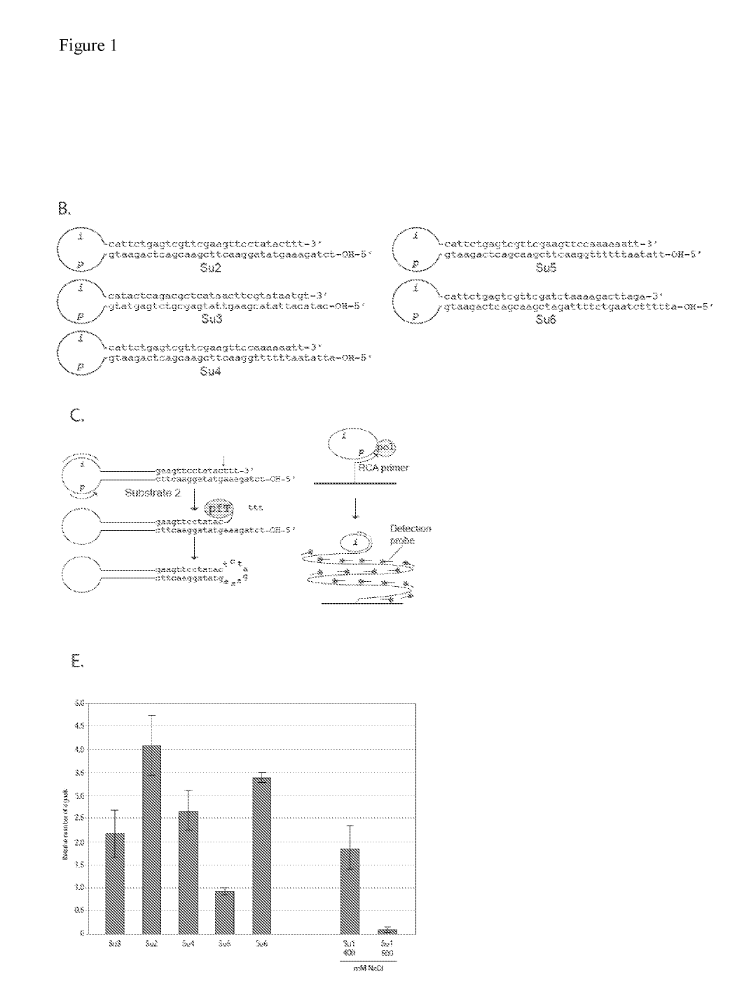

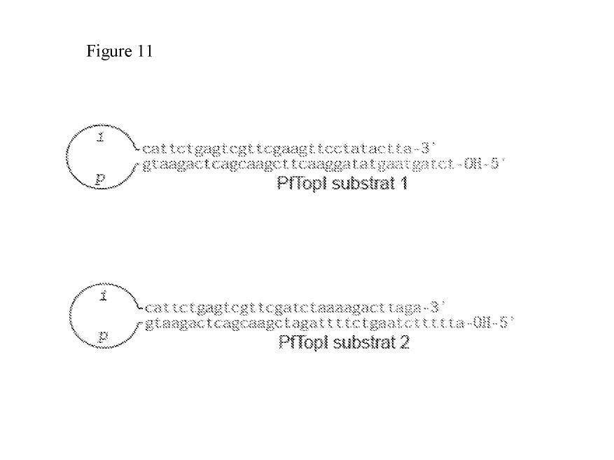

In another embodiment, the method and/or kit comprises a nucleic acid substrate comprising the sequence TCTAGTAAG-(N)ₓ-CTTA or ATTTTTCTA-(N)ₓ-TAGA, where N is A, T, C, or G, and x is between 5 and 500 (SEQ ID NOs: 18 or 19). More specifically, the number of nucleotides between the two invariable regions (x) is 5-400, such as 5-300, for example 5-200, such as 10-200, such as 30-150, for example 40-130, such as 50-120, such as 60-100 nucleotides. In another embodiment, the nucleic acid substrate comprises a sequence, with at least 30%, 40%, 50%, 60%, 70%, 80%, such as at least 90%, for example at least 91%, 92%, 93%, 94%, 95%, 96%, 97%, 98%, such as at least 99% identity to any one of SEQ ID NOs: 8-17, while also comprising the sequence TCTAGTAAG-(N)x-CTTA or ATTTTTCTA-(N)x-TAGA, where N is A, T, C, or G, and x is between 5 and 500, such as described above (SEQ ID NOs: 18 or 19). However, the number of nucleotides between the two non-variable regions may also be over 500, however, this is less preferred, because the size of the substrate might reduce the efficiency of detection of processed substrate. Importantly, substrates of this type preferably folds into a double stranded structure by forming a hairpin structure, where the two non-variable regions forms base pairs over a certain region, cf. for example FIGS. 1 and 11. Thus, in a preferred embodiment, the nucleotides in the region defined as (N)x form a hairpin structure, i.e. stem-loop intramolecular base pairing, wherein at least 5, but more preferably at least 10, such as at least 15, or at least 20 consecutive nucleotides form intramolecular base pairing with complementary nucleotides of the same nucleic acid molecule.

In yet another embodiment, the method and/or kit comprises a nucleic acid substrate comprising a sequence selected from any one of SEQ ID NO: 5-7, a sequence at least 30%, 40%, 50%, 60%, 70%, 80%, such as at least 90%, for example at least 91%, 92%, 93%, 94%, 95%, 96%, 97%, 98%, such as at least 99% identical thereto, or a part of at least 5 consecutive nucleotides, such as at least 10, 15, 20, 30, 40, 50, 60, 70, 80, 90, such as at least 100 consecutive nucleotides, of any of said sequences. In this case, the microorganism is selected from the Mycobacterium genus, for example the microorganism is Mycobacterium tuberculosis.

More specifically, the method and/or kit may comprise a nucleic acid substrate comprising SEQ ID NO: 7, and in one embodiment, the method and/or kit comprises a nucleic acid substrate comprising a sequence, with at least 30%, 40%, 50%, 60%, 70%, 80%, such as at least 90%, for example at least 91%, 92%, 93%, 94%, 95%, 96%, 97%, 98%, such as at least 99% identity to any one of SEQ ID NO: 5 and 6, while also comprising SEQ ID NO: 7.

### Sample

A “sample” according to the present invention is any suitable biological or non-biological sample. The choice of sample depends on the specific cell, cell type, microorganism, disease or infectious disorder to be determined as well as the detection method, and will be appreciated by those of skill in the art.

An examples of a non-biological sample is water, such as drinking water, which is subjected to analysis for the detection of contamination with microorganisms, such as infectious agents, for example pathogenic bacteria or parasitic microorganism, e.g. Mycobacteria or Plasmodium. However, also other non-biological samples are applicable, in any case a sample or area should be tested for the absence of specific microorganisms or other cell types. For example, facilities used for food production, conveyor belts etc.

The sample may originate, be obtained or isolated from any source, which is of interest for detection of specific cell types or microorganisms. The biological sample may originate, be obtained or isolated from any subject of the animal kingdom, depending on the intended use of the method of the invention. For example, the sample may originate, be obtained or isolated from any subject of vertebrates, such as mammals, reptiles, fish, birds, and amphibians. In a preferred embodiment, the biological sample is in a preferred embodiment, isolated or originating or obtained from a mammalian subject, such as a human being or a bovine subject. In other non-limiting examples, the sample is a sample originating, obtained or isolated from a ruminant, a ferret, a badger, a rodent, an elephant, a bird, a pig, a deer, a coyote, a camel, a puma, a fish, a dog, a cat, a non-human primate or a human. In a preferred embodiment, the sample is originating or obtained from a human being; i.e. the sample is a human sample. In another embodiment, the sample is originating, isolated or obtained from a non-human animal; i.e. the sample is a non-human animal sample. In one preferred embodiment, the sample is originating or obtained from a bovine subject; i.e. the sample is a bovine sample.

When the sample of the invention is a biological sample, the sample comprises cells, which originate from the subject from which the sample is isolated. Thus, in one embodiment, the sample comprises eukaryotic cells, such as mammalian, reptile, fish, bird or amphibian cells. The sample may however, also comprise specific cells of said subject. For example, the sample in one embodiment comprises cancer cells, such as cancer cells isolated from a human being.

In one embodiment the sample is a blood sample, a tissue sample, a secretion sample, semen, ovum, hairs, nails, tears, urine, biopsy or faeces. A convenient sample type is a blood sample. The blood sample includes any fraction of blood, such as blood plasma or blood serum, sputum, urine, cell smear.

However, the sample of the invention may also be a tissue sample, such as a sample of a tissue selected from the group consisting of skin, epidermis, dermis, hypodermis, breast, fat, thymus, gut, small intestine, large intestine, stomach, muscle, pancreas, heart muscle, skeletal muscle, smooth muscle, liver, lung, brain, cornea and tumours, ovarian tissue, uterine tissue, colon tissue, prostate tissue, lung tissue, renal tissue, thymus tissue, testis tissue, hematopoietic tissue, bone marrow, urogenital tissue, expiration air, stem cells, including cancer stem cells, biopsies, and cerebrospinal fluid. In one embodiment, the sample is blood plasma, blood serum, sputum, urine, cell smear, faeces, cerebrospinal fluid, or a biopsy.

However, in another important application of the methods of the present invention, the sample is obtained from any source of human or animal consumption, such as food or feed; i.e. the sample is a food or feed sample. In another embodiment, the sample is water, such as drinking water and domestic water.

### Microorganism

As explained herein above, the present invention relates to a method of identifying a microorganism expressing a specific enzyme, such as a type I topoisomerase-expressing microorganism in a sample, as well as a method of determining a disease in a subject based on identifying a microorganism in a sample. A microorganism of the present invention encompasses any pathogenic and/or parasitic agent, so for example the microorganism is a pathogenic microorganism, and in another example, the microorganism is a parasitic microorganism. The microorganism is for example a virus, a bacteria, a protozoa, a fungus, a mould, an amoeba or a parasitic worm.

The present invention relates to a method for identifying a microorganism as well as methods and compounds for treating an infectious disorder, which is caused by a microorganism. The invention also provides kits for use in such methods, where the kits comprise at least one nucleic acid substrate targeted by a type I topoisomerase of a microorganism and means for detection of nucleic acid substrate processed by said topoisomerase. The microorganism of the invention is thus, mostly, a pathogenic microorganism. Microorganism includes bacteria and viruses.

The microorganism identified by the method of the present invention is for example involved in and/or is the causative agent in one or more infectious disorders. The microorganism is for example involved in tuberculosis, malaria, toxoplasmosis or Lyme disease/borreliosis (Borrelia). In one embodiment, the microorganism is Plasmodium falciparum, or Mycobacterium tuberculosis, enterobacteria, enterococci, corynebacteria, Salmonella spp, Mycobacterium avium sp. paratuberculosis, Brachyspira hyodysenteriae, Lawsonia intracellularis, campylobacter spp., clostridia, coronavirus, rotavirus, torovirus, calicivirus, astrovirus, canine parvovirus, coccidia and cryptosporidia, E. coli, Salmonella spp, Yersinia spp., including Yersinia enterocolitica, Mycobacterium avium ssp. paratuberculosis, Coxiella burnetti, rotavirus, coronavirus, calicivirus, bovine virus diarrhoea virus, bovine herpes virus, rinderpest virus, coccidia, and cryptosporidia, Salmonella spp, Lawsonia intracellularis, Campylobacter spp, Enteropathogenic E. coli, Brachyspira spp including Brachyspira hyodysenteria, Clostridium spp, rotavirus, sappovirus, norovirus, and coronavirus, Salmonella spp, Camphylobacter spp., Norovirus, rotavirus, Vibrio spp. including Vibrio cholera, Shigella spp., Helicobacter spp., coccidia or cryptosporidia.

If the microorganism is a bacterium, the microorganism is selected from Eubacteria, or is selected from Actinobacteria, or is selected from Actinomycetes, or is selected from Corynebacterineae, or is selected from Mycobacteriaceae, or is selected from Mycobacteria. In one embodiment, the microorganism is selected from the Mycobacterium Genus, and a more specific embodiment, the microorganism is Mycobacterium tuberculosis or Mycobacterium bovis

In one embodiment, the microorganism of the methods and kits of the present invention is selected from Eukaryotes, or is selected from Alveolates, or is selected from Apicomplexans/sporozoans, or is selected from Haematozoans, or is selected from Haemosporidians, or is selected from Plasmodiidans, or is selected from Plasmodium. In a preferred embodiment, the microorganism belongs to the Plasmodium genus. The microorganism is for example selected from the following species: Plasmodium clelandi, Plasmodium draconis, Plasmodium lionatum, Plasmodium saurocordatum, Plasmodium vastator, Plasmodium juxtanucleare, Plasmodium basilisci, Plasmodium clelandi, Plasmodium lygosomae, Plasmodium mabuiae, Plasmodium minasense, Plasmodium rhadinurum, Plasmodium volans, Plasmodium anasum, Plasmodium circumflexum, Plasmodium dissanaikei, Plasmodium durae, Plasmodium fallax, Plasmodium formosanum, Plasmodium gabaldoni, Plasmodium garnhami, Plasmodium gundersi, Plasmodium hegneri, Plasmodium lophurae, Plasmodium pedioecetii, Plasmodium pinnotti, Plasmodium polare, Plasmodium cathemerium, Plasmodium coggeshalli, Plasmodium coturnixi, Plasmodium elongatum, Plasmodium gallinaceum, Plasmodium giovannolai, Plasmodium lutzi, Plasmodium matutinum, Plasmodium paddae, Plasmodium parvulum, Plasmodium relictum, Plasmodium tejera, Plasmodium elongatum, Plasmodium hermani, Plasmodium floridense, Plasmodium tropiduri, Plasmodium billbrayi, Plasmodium billcollinsi, Plasmodium falciparum, Plasmodium gaboni, Plasmodium reichenowi, Plasmodium pessoai, Plasmodium tomodoni, Plasmodium wenyoni, Plasmodium ashfordi, Plasmodium bertii, Plasmodium bambusicolai, Plasmodium columbae, Plasmodium corradettii, Plasmodium dissanaikei, Plasmodium globularis, Plasmodium hexamerium, Plasmodium jiangi, Plasmodium kempi, Plasmodium lucens, Plasmodium megaglobularis, Plasmodium multivacuolaris, Plasmodium nucleophilum, Plasmodium papernai, Plasmodium parahexamerium, Plasmodium paranucleophilum, Plasmodium rouxi, Plasmodium vaughani, Plasmodium dominicum, Plasmodium chiricahuae, Plasmodium mexicanum, Plasmodium pifanoi, Plasmodium bouillize, Plasmodium brasilianum, Plasmodium cercopitheci, Plasmodium coatneyi, Plasmodium cynomolgi, Plasmodium eylesi, Plasmodium fieldi, Plasmodium fragile, Plasmodium georgesi, Plasmodium girardi, Plasmodium gonderi, Plasmodium gora, Plasmodium gorb, Plasmodium inui, Plasmodium jefferyi, Plasmodium joyeuxi, Plasmodium knowlei, Plasmodium hyobati, Plasmodium malariae, Plasmodium ovale, Plasmodium petersi, Plasmodium pitheci, Plasmodium rhodiani, Plasmodium schweitzi, Plasmodium semiovale, Plasmodium semnopitheci, Plasmodium silvaticum, Plasmodium simium, Plasmodium vivax, Plasmodium youngi, Plasmodium achiotense, Plasmodium adunyinkai, Plasmodium aeuminatum, Plasmodium agamae, Plasmodium balli, Plasmodium beltrani, Plasmodium brumpti, Plasmodium cnemidophori, Plasmodium diploglossi, Plasmodium giganteum, Plasmodium heischi, Plasmodium josephinae, Plasmodium pelaezi, Plasmodium zonuriae, Plasmodium achromaticum, Plasmodium aegyptensis, Plasmodium anomaluri, Plasmodium atheruri, Plasmodium berghei, Plasmodium booliati, Plasmodium brodeni, Plasmodium bubalis, Plasmodium bucki, Plasmodium caprae, Plasmodium cephalophi, Plasmodium chabaudi, Plasmodium coulangesi, Plasmodium cyclopsi, Plasmodium foleyi, Plasmodium girardi, Plasmodium incertae, Plasmodium inopinatum, Plasmodium landauae, Plasmodium lemuris, Plasmodium melanipherum, Plasmodium narayani, Plasmodium odocoilei, Plasmodium percygarnhami, Plasmodium pulmophilium, Plasmodium sandoshami, Plasmodium traguli, Plasmodium tyrio, Plasmodium uilenbergi, Plasmodium vinckei, Plasmodium watteni and Plasmodium yoelli. Most preferred, the microorganism is Plasmodium falciparum, which is a causative agent of human Malaria.

In another preferred embodiment, the microorganism belongs to the Mycobacterium genus. The microorganism is for example selected from the Mycobacterium tuberculosis complex (MTBC), the members of which are causative agents of human and animal tuberculosis. Species in this complex include: M. tuberculosis, M. bovis, M. bovis BCG, M. africanum, M. canetti, M. caprae, M. microti, and M. pinnipedii. Most preferrably, the microorganism is Mycobacterium tuberculosis, which is the major cause of human tuberculosis.

### Detection

According to the methods of the present invention, an enzyme, a cell, cell type or microorganism is identified in a sample by detecting a nucleic acid substrate which is targeted by an enzyme, such as a type I topoisomerase of a sample or the specific cell, cell type or microorganism. As described above, type I topoisomerase targets double stranded nucleic acid molecules by binding a region of said nucleic acid molecule and cleaving a single strand of the duplex. A nucleic acid substrate, which has been targeted by type I topoisomerase may thus be detected by identifying those nucleic acid substrates in the sample that have been cleaved. The nucleic acid substrate is thus, preferably targeted by an enzyme, such as topoisomerase I, of the microorganisms only, and not by other enzymes/topoisomerase I-activities of the sample, such as native topoisomerases of the subject tested for enzymes, cells or cell types, or microorganisms or infectious disorders such as malaria and/or tuberculosis.

Detection of cleaved and uncleaved (targeted and untargeted) nucleic acid substrates may be performed by any suitable method available. Detection is for example obtained by southern blotting, polymerase chain reaction, RT-PCR, qPCR, RFLD, primer extension, DNA array technology, a linear amplification technique, isothermal amplification, and/or rolling circle amplification. In a preferred embodiment, the nucleic acid substrate is detected by rolling circle amplification, for example by a method as described in WO 2008/148392.

Processed nucleic acid substrate is in a preferred embodiment detected by rolling circle amplification performed by

i. providing at least one oligonucleotide primer, which is capable of hybridizing to circularized nucleic acid substrate,
ii. hybridizing the at least one oligonucleotide primer to the circularized nucleic acid substrate,
iii. providing a nucleic acid polymerase and nucleotides
iv. generating a rolling circle amplification product by extending the at least one oligonucleotide primer using the circularized nucleic acid substrate as template, and
v. detecting the rolling circle amplification product.

In certain aspects, a detection assay can be a quantitative amplification assay, such as quantitative PCR (qPCT) or quantitative RT-PCR (qRT-PCR). Other methods include hybridization assays, such as array hybridization assays or solution hybridization assays. The nucleic acid substrate may be labelled, and/or hybridized to one or more nucleic acid probes, and detected via the respective label.

In a convenient setup of the detection methods of the present invention, a simple portable readout devices or even with colorimetric readout visible for the naked eye, adapting the biosensor for at-place-of-care diagnosis.

### Primers and Probes

Detection of nucleic acid substrate both processed and non-processed substrates may be obtained by use of different tailored primers and probes, preferably oligonucleotide primers and/or probes. The primers and probes should be able to bind to the nucleic acid substrate either directly or indirectly. The sequence of the oligonucleotide primers and probes should of course be complementary to the substrate sequence and the general design of such oligonucleotide primers and probes are well known to those of skill in the art. Oligonucleotide primers and probes of any suitable lengths are within the scope of the invention, for example oligonucleotides of 5-300 nucleotides, such as 10-200, 20-100, or 20-50 consecutive nucleotides.

In the present invention, primers are primarily used for polymerisation/extension catalysed by a polymerase, preferably a DNA polymerase, such as phi polymerase, or any other suitable polymerase, where the primers hybridize to the nucleic acid substrate of the invention. Thus, the primers of the methods and kits of the present invention are preferably capable of hybridizing to a processed substrate. However, primers hybridizing to unprocessed substrate may also be employed, for example in positive control reactions. The primer may span the processed nucleotide position of the processed nucleic acid substrate, thereby only supporting amplification of processed substrates. However, the primers may also be designed to hybridize to other positions of the nucleic acid substrate, since in certain embodiments, targeted nucleic acid substrate is circularized by topoisomerase processing, thereby serving as a template for rolling circle amplification using a primer, which hybridize anywhere in the substrate sequence. For this reason, oligonucleotide primers hybridizing anywhere in the nucleic acid substrate are within the scope of the present invention.

For more convenient manipulation and detection, the at least one oligonucleotide primer is in one embodiment coupled to a magnetic bead. In this case, primers and/or amplification product may be transferred or otherwise manipulated using magnets/magnetic fields. The methods and kits of the invention may thus comprise primers and/or probes coupled to magnetic beads and/or magnets/magnetic fields. In further embodiments, the methods and/or kits may comprise a nucleic acid polymerase and/or nucleotides, for use in amplification of a processed substrate.

In another preferred embodiment, the primers are coated on a glass slide, which is contacted with the retained droplets, which comprise processed and/or non-processed substrate.

In one embodiment, the oligonucleotide primer or probe of the methods and/or kits comprise a sequence of at least 5 consecutive complementary nucleotides, such as at least 10, 15, 20, 30, 40, 50, 60, 70, 80, 90, such as at least 100 consecutive complementary nucleotides selected from any region of any of SEQ ID NO: 5-32, and/or any sequence at least 30%, 40%, 50%, 60%, 70%, 80%, such as at least 90%, for example at least 91%, 92%, 93%, 94%, 95%, 96%, 97%, 98%, such as at least 99% identical thereto.

In one embodiment, the oligonucleotide primer or probe of the methods and/or kits is SEQ ID NO: 20, 21, 22, 23, 24, or 27-32. For example, the oligonucleotide primer of kits or methods of the present invention is SEQ ID NO: 23 or 24, and the oligonucleotide probe of kits or methods of the present invention is SEQ ID NO: 20, 21, or 22.

In a specific example, the probe of the methods and/or kits of the present invention comprises a sequence according to SEQ ID NO: 20-22, or a sequence at least 30%, 40%, 50%, 60%, 70%, 80%, such as at least 90%, for example at least 91%, 92%, 93%, 94%, 95%, 96%, 97%, 98%, such as at least 99% identical thereto, or a part of at least 5 consecutive nucleotides, such as at least 6, 7, 8, 9, 10, 11, 12, 13, 14, 15, 16, 17, 18, 19, 20, 30, 40, 50, such as at least 60 consecutive nucleotides, of any of said sequences.

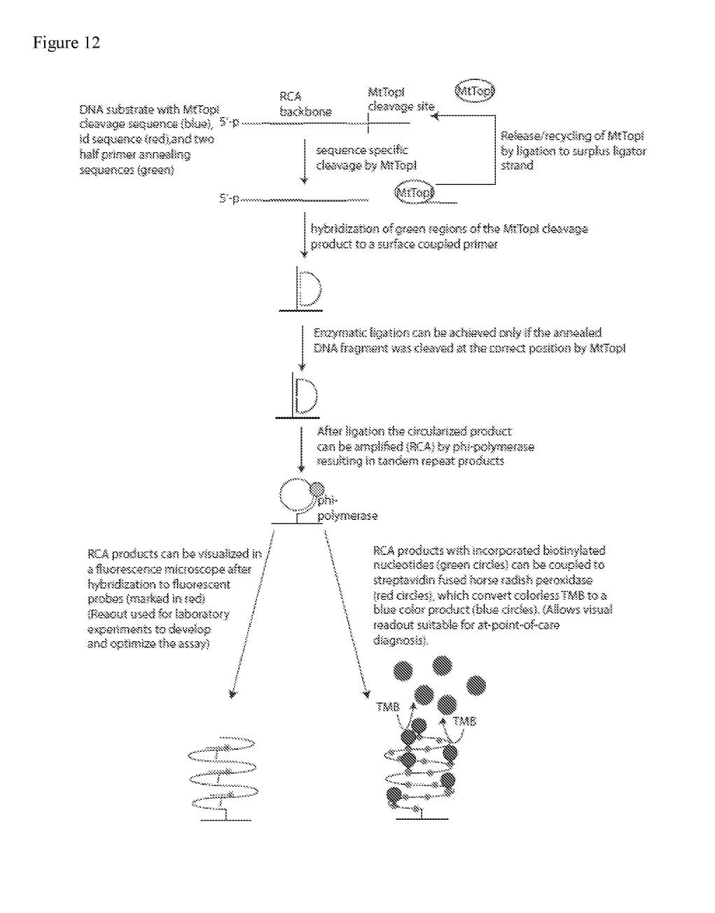

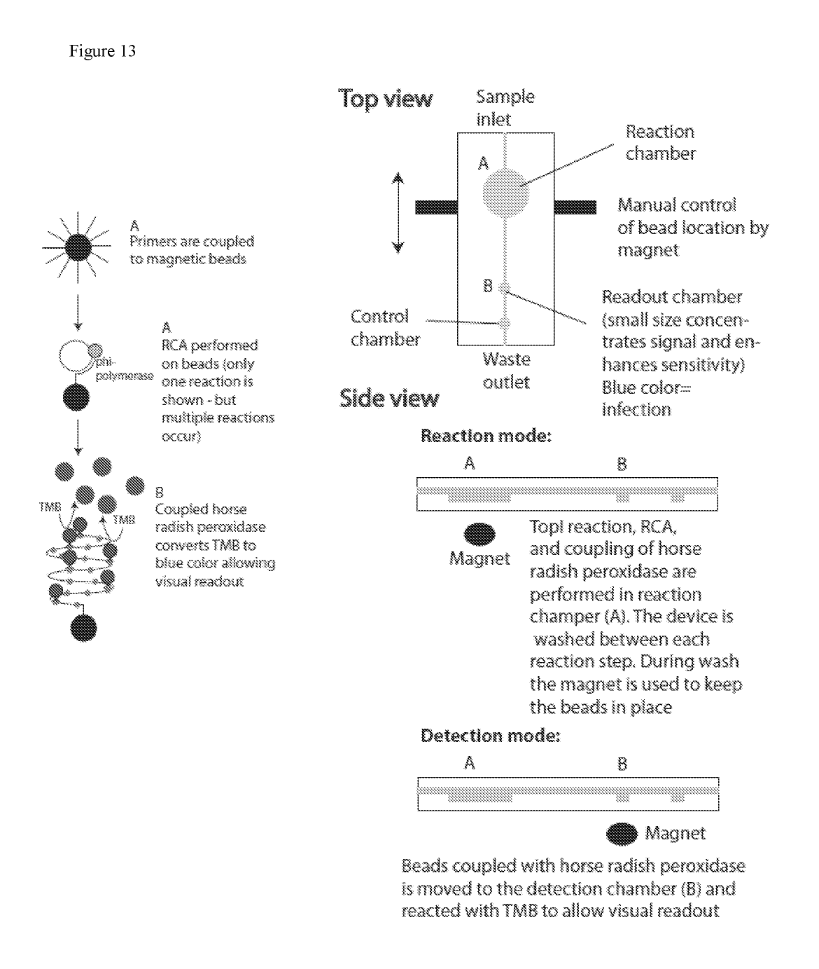

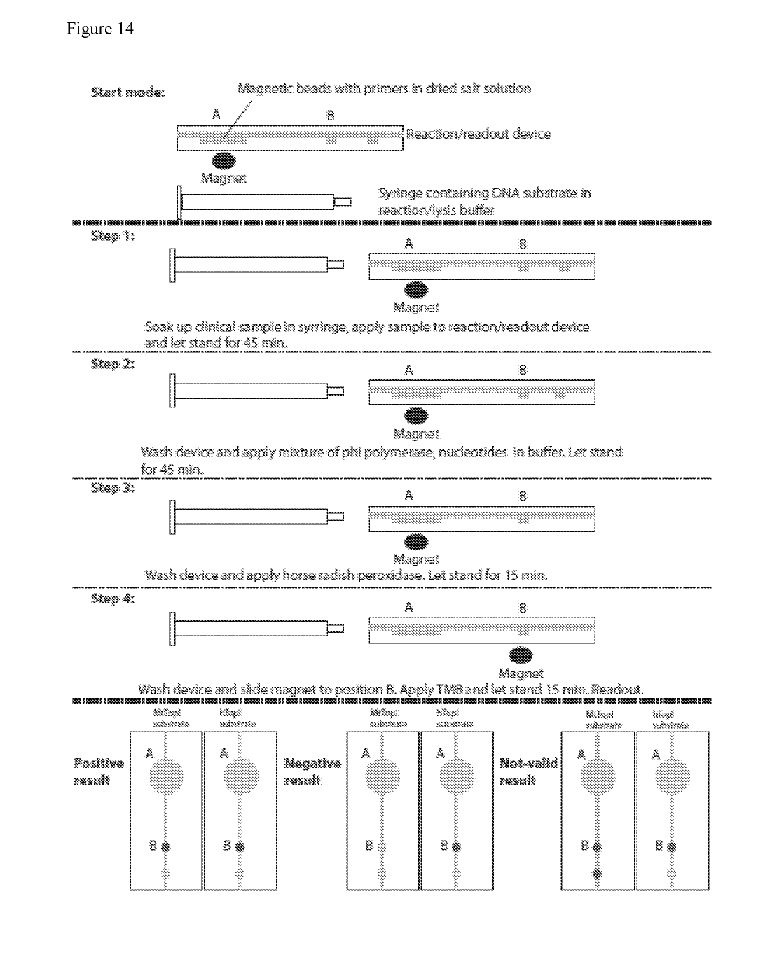

In a user friendly set up of the methods and kits of the present invention, nucleic acid substrate, oligonucleotide primer, oligonucleotide probe, nucleic acid polymerase and/or nucleotides is immobilized on a solid support. Wth the oligonucleotide primer immobilized on a solid support, an amplification product, such as a rolling circle amplification product, is confined to a specific location, which allows the product to be manipulated, transferred and/or detected by washing and probe hybridization, cf. FIGS. 1C, 4B, 10, 12, 13, 14, 21 and 26.

The choice of solid support depends on the specific approach of the methods and kits of the invention and a range of possible solutions are available to those of skill in the art. The solid support is for example a glass surface, or a magnetic bead. In one embodiment, the nucleic acid substrate, oligonucleotide primer, oligonucleotide probe, nucleic acid polymerase and/or nucleotides is immobilized on a glass slide. In particular, the oligonucleotide primer of the methods and kits of the present invention is immobilized on such a glass slide.

### Detection and Visualization

The processed nucleic acid substrate is then detected, for example by detection of an amplification product generated on the basis of processed topoisomerase substrate, such as rolling circle amplification product. Detection is preferably performed by observation of a visual signal, while radioactive signals could also be employed, which can also be visualized by radioautography. The processed substrate may for example be detected by visualizing a rolling circle amplification product. Thus, any suitable coloring agent may be employed for this purpose.

In one embodiment, the nucleotides comprised in the kit of the invention or used in the method comprise one or more detectable labels, such as a fluorophore and/or radioactively labelled nucleotides. In this case, the rolling circle amplification product is detected via its incorporation of such nucleotides comprising one or more detectable labels.

In another embodiment, detection is obtained by use of at least one nucleic acid probe capable of hybridizing to the nucleic acid substrate, the processed nucleic acid substrate and/or the nucleic acid amplification product. In this case, the processed nucleic acid substrate or amplification product, such as rolling circle amplification product, is detected by hybridization of a labelled nucleic acid probe to one or multiple sites of the processed nucleic acid substrate or amplification product, such as the rolling circle amplification product. The probe is for example labelled with one or more fluorescent dyes, radioactive nucleotides and/or biotinylated nucleotides. The nucleic acid probe, preferably comprise at least one detectable label. For example, the probe is labelled with one or more enzymes, fluorescent dyes, radioactive nucleotides and/or biotinylated nucleotides. In a preferred embodiment, the probe is coupled to an enzyme, such as an enzyme, which is capable of converting a substrate into a detectable product. The enzyme is for example fused with streptavidin, thereby enabling it to be coupled via biotin. Thus in a preferred embodiment of the methods and kits, the enzyme is fused with streptavidin, and coupled to the nucleic acid probe via interaction with said biotinylated nucleotides incorporated in the nucleic acid probe. The enzyme is any enzyme with an easily detectable activity. In one example, the enzyme is horse-radish peroxidase. In this case, the method and/or kit may further comprise TMB (3,3′,5,5′-Tetramethylbenzidine) or functional equivalents thereof as a substrate for the enzyme. In the case of other enzymes included in the kit or employed in the method of the invention, the kit or method may also further comprise/employ any suitable substrate for the respective enzyme.

### Specific Applications

The technology of the methods and kits of the present invention may be employed for identifying any microorganism and/or any infection in any subject, including humans, non-human animals, such as house-hold stocks, and plants, as described herein above. Other applications of the method and kit include the identification of microorganisms in the contamination of food and drinking water.

### Biosensor for Detection of Tuberculosis

Topoisomerase I from the tuberculosis-causing pathogen M. tuberculosis (MtTopI) belongs to the type IA family of topoisomerases, which normally require Mg2+ for activity. However, MtTopI only requires this cofactor during the ligation step of catalysis and not during cleavage. Therefore, MtTopI cleavage can be detected even in crude biological samples, which are depleted for Mg2+. Moreover, MtTopI cleaves single stranded DNA in a sequence specific manner, which allows the specific cleavage activity of MtTopI to be distinguished from other nucleases. Hence, a single stranded nucleic acid substrate is provided to the sample to be tested for Mycobacterium tuberculosis, and that nucleic acid substrate is then upon cleavage by MtTopI converted to a well-defined product with a specific sequence. This product can then be detected by any suitable method, as described herein above. Thus, in a preferred embodiment of the method of the present invention, the sample is depleted for divalent cations, and/or the kit comprises an agent for depletion of cations. This is for example specifically relevant for the identification of microorganisms, which express a type I topoisomerase that do not require divalent cations for processing a nucleic acid substrate by cleavage and/or ligation, such as MtTopI (Mycobacterium tuberculosis topoisomerase I).

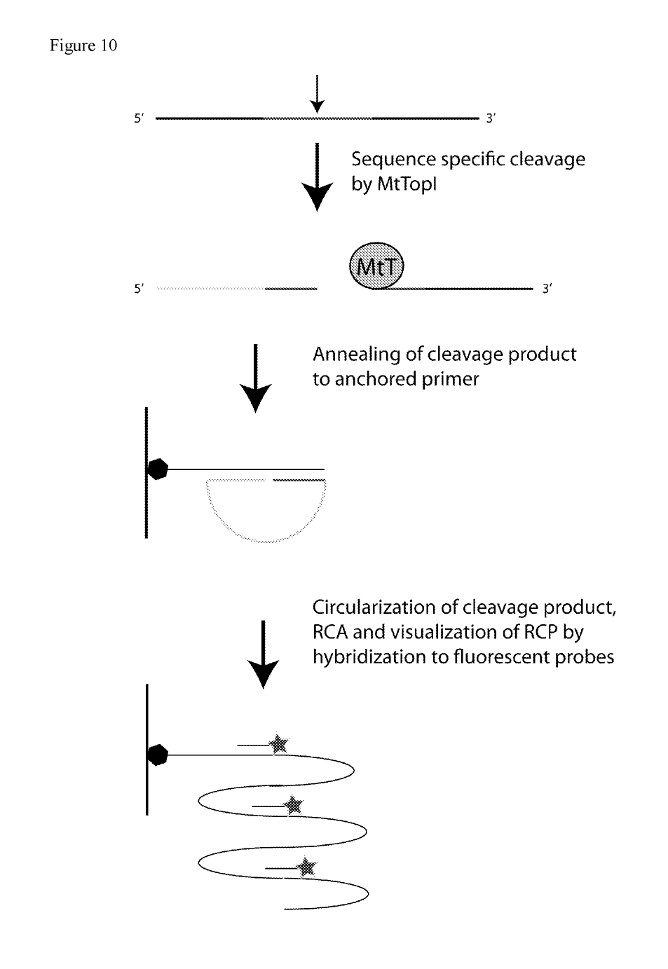

For example, the cleavage product is hybridized to a primer anchored to a glass surface and circularized by a DNA ligase (in the presence of Mg2+) after cell remains have been washed away. The generated circle can now serve a template for RCA and the products visualized by hybridization to specific fluorescent probes (cf. FIG. 10).

### Medical Use

The technology may be employed for identifying any microorganism and/or any infection in any subject, including humans, non-human animals, such as house-hold stocks, and plants. Other applications of the method include the identification of microorganisms in the contamination of food and drinking water.

The compositions, kits and methods provided herein are also intended for medical use. Specifically, the compositions, methods and medicaments are provided for identifying a microorganism as defined herein. The microorganisms are preferably pathogenic microorganism, i.e. are among the causative agents of an infectious disorder. Therefore, the compositions, kits and methods of the present invention are also provided for the diagnosis of infectious disorders as described herein, in particular for the diagnosis of malaria and/or tuberculosis

In a further aspect, the present invention relates to a method of identifying novel lead compounds for treatment of infectious disorders. To this end, a method is provided for evaluating the effect of an agent on a pathogenic microorganism. Based on the compounds identified in such method, the present invention also relates to such agents for use in the treatment of an infectious disorder, in particular malaria and/or tuberculosis.

### Diagnosis

So in one main medical aspect, the present invention relates to a method of determining an infectious disorder in a subject, in particular in a human being. The method comprises identifying a microorganism in a sample from said subject by a method of the present invention. The presence of microorganism in said sample is then indicative of said infectious disorder, because the microorganism is a causative agent of that particular infectious disorder. The infectious disorder determined according to the present invention is for example without limitation tuberculosis, malaria, toxoplasmosis or Lyme disease/borreliosis (Borrelia).

A large number of microorganisms are known to be associated as causative agents with certain infectious disorders. For example, Plasmodium falciparum is known to be a causative agent of malaria, and Mycobacterium tuberculosis is known as a major causative agent of human tuberculosis.

The provided method for identifying a microorganism in a sample from subject generally comprises

i. providing the sample
ii. providing an at least partly double stranded nucleic acid substrate targeted by type I topoisomerase of said microorganism,
iii. mixing the sample of step i. with the nucleic acid substrate of step ii.
iv. detecting nucleic acid substrate targeted by type I topoisomerase of said microorganism,
wherein the presence of nucleic acid substrate targeted by type I topoisomerase of said microorganism is indicative of said microorganism.

Generally, the present invention provides a method of determining a disease in a subject, said method comprising identifying a microorganism in a sample from said subject by a method of the present invention as defined herein above, wherein the presence of said microorganism in said sample is indicative of said disease. The method of the invention is here generally understood as a method of identifying a type I topoisomerase-expressing microorganism in a sample, said method comprising

i. providing the sample
ii. providing a nucleic acid substrate targeted by a type I topoisomerase of said microorganism,
iii. bringing the sample of step i. in contact with the nucleic acid substrate of step ii.
iv. detecting nucleic acid substrate processed by said type I topoisomerase of said microorganism,
wherein the presence of processed nucleic acid substrate is indicative of said microorganism.

The subject diagnosed for a disease according to the method of determining a disease is not necessarily limited to any specific group, family or class of organisms. In one embodiment, however, the subject is a ruminant, a bovine, a ferret, a badger, a rodent, an elephant, a bird, a pig, a deer, a coyote, a camel, a puma, a fish, a dog, a cat, a non-human primate or a human. In a preferred embodiment, the subject is a human subject. Human subjects are for example preferred when testing for human diseases, such as human tuberculosis, and/or malaria. In another embodiment, the subject is a bovine subject; for example when testing for bovine diseases, such as bovine tuberculosis. The disease is any disease of interest, as explained elsewhere herein, for example the disease is an infectious and/or a parasitic disease, and for example the infectious disease is malaria.

Also, the microorganisms identified are explained elsewhere herein; for example, the microorganism is selected from the Plasmodium and/or Mycobacterium genus. In one preferred embodiment, the parasitic disease is malaria and/or the microorganism is selected from the Plasmodium genus, for example, the microorganism is Plasmodium falciparum.

In one embodiment, the infectious disease is human and/or bovine tuberculosis, and/or the microorganism is selected from the Mycobacterium genus, for example Mycobacterium tuberculosis.

The diagnostic applications of the present invention may be practised in any suitable and practical setup or machinery, which utilizes the system's sensitivity, simplicity and short reaction time. For example, the method may be used in advanced equipment for single cell, single molecule detection, for ultra-sensitive detection of infection sources.

However, in a particularly preferred embodiment, the diagnostic methods are performed in the style of stick tests/dipsticks. A testing dipstick is for example made of paper or cardboard and is impregnated with the reagents required to perform the reaction of the invention. The readout of a dipstick test is preferably presented by a changing color. In this way, dipsticks can be used to test for a variety of liquid samples for the presence of a specific microorganism, and the dipstick can then be employed in easy and efficient diagnosis of infectious disorders, such as any infectious disorder according to the present invention.

### Method for Drug Discovery

The species-specific enzyme reactions, particular type I topoisomerase activity, which serve to modify nucleic acids/DNA, and thereby are used for identification of a cell, cell type or microorganism according to the present invention, are generally essential for these cells, cell types or microorganisms since they are part of DNA metabolism. Any compound capable of specifically blocking, inhibiting or down-regulating the activity of these species-specific enzymes may be used as therapeutic against such cells, cell types (e.g. cancer cells) or microorganisms and/or infectious disorders caused by such microorganisms. The methodology of the present invention can be applied directly to the testing of drugs known for their selective action on specific enzymatic processes in the relevant microorganism or cell type. Moreover, the methodology can be used for design of new small molecule drugs against nucleic acids modifying enzyme systems of specific cells, cell types or infectious or parasitic microorganisms.

Accordingly, the teaching of the present invention may also be employed in drug discovery, because the method provided herein for determining the presence of microorganisms via the presence of a specific enzyme, such as type I topoisomerase, activity, can be used for evaluating the effect of a candidate drug on a cell, cell type or microorganism. Any such drug candidates, which display an inhibitory effect on the presence of the microorganism and/or on the activity of the enzyme, e.g. topoisomerase activity, of said cell, cell type or microorganism is a suitable drug for treatment of said cell or cell type (for example cancer cells), or an infectious disorder associated with that microorganism.

Thus, the present invention in one aspect relates to a method for evaluating the effect of a chemical agent on a cell, cell type or microorganism in a sample, said method comprising

i. providing a sample
ii. providing a nucleic acid substrate targeted by an enzyme, such as a type I topoisomerase, of said cell, cell type or microorganism,
iii. providing a chemical agent,
iv. combining the sample of step i. and the nucleic acid substrate of step ii. with or without the agent of step iii.
v. detecting nucleic acid substrate targeted by an enzyme, such as type I topoisomerase of said cell, cell type or microorganism with or without the agent, wherein a chemical agent capable of reducing the amount of targeted nucleic acid substrate has an inhibitory effect on said cell, cell type or microorganism.

In this method, the cell, cell type or microorganism, sample, nucleic acid substrate, enzyme, such as type I topoisomerase, and/or detection is as defined in any of the preceding claims.

### Treatment

In yet another aspect, the present invention provides an agent, a composition, a use, or a method for treatment of an infectious disorder, based on a candidate drug identified by a method provided herein. Thus, in a further aspect the present invention relates to a method of treating, preventing or ameliorating an infectious disorder, said method comprising administering an agent identified by a method of the present invention to a subject in need thereof.

Similarly, the invention also provides for an agent identified by the method of the present invention and/or a pharmaceutical composition comprising such agent for use in the treatment, prevention or amelioration of an infectious disorder.

### EXAMPLES

### Example 1

### Development of a Novel Plasmodium falciparum Topoisomerase I Specific Biosensor for Diagnosis of Malaria

This example relates to a DNA based biosensor suitable for at-point-of-care diagnosis of malaria. In this setup, specific detection of malaria parasites in crude blood samples is facilitated by the conversion of single Plasmodium falciparum topoisomerase I (pfTopI) mediated cleavage-ligation events, happening within nanometer dimensions, to micrometer-sized products readily detectable at the single molecule level in a fluorescence microscope.

One challenge of detecting enzymatic products is that only few enzymatic products are readily detectable without the use of sophisticated equipment and even then, most products can be detected only when produced in high numbers. For clinically relevant identification of pathogens based on species specific enzymatic activities it is, therefore, necessary to have detection systems that overcome these challenges.

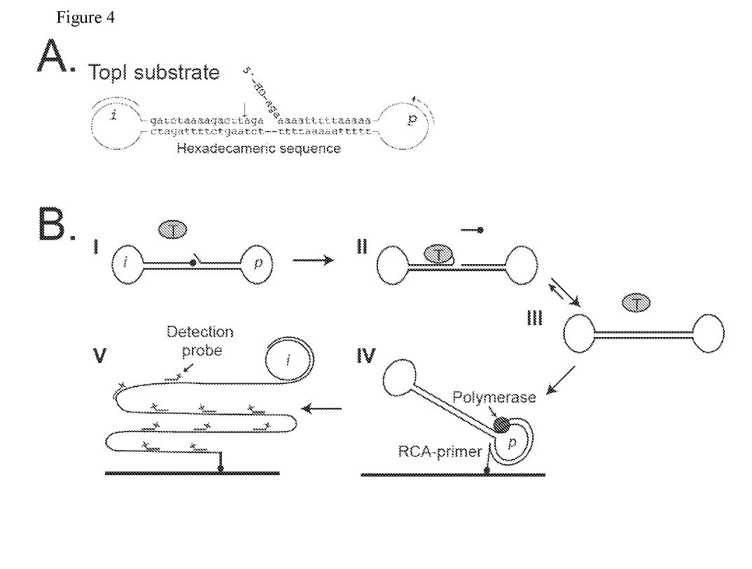

The enzymes Topoisomerase I (hTopI), Flp and Cre all introduce single strand cuts in DNA followed by subsequent ligation of the generated nick in a reaction that involves the formation of a covalent enzyme-DNA cleavage intermediate. This reaction may be utilized to convert self-folding oligonucleotide substrates to closed DNA circles, which subsequently were subjected to Rolling Circle Amplification (RCA) leading to products (RCP) consisting of ˜10³ tandem repeats of a sequence complementary to the DNA circles. These RCPs can be visualised at the single-molecule level in a fluorescence microscope by annealing to fluorescent-labelled probes giving rise to one fluorescent spot for each RCP (see FIG. 4 for schematic illustration of the assay). Since the assay involves no thermal cycling, each RCP represents one closed DNA circle, which in turn represented a single cleavage-ligation event. Hence, this assay allows the detection of TopI, Flp or Cre activity at the single cleavage-ligation event level.

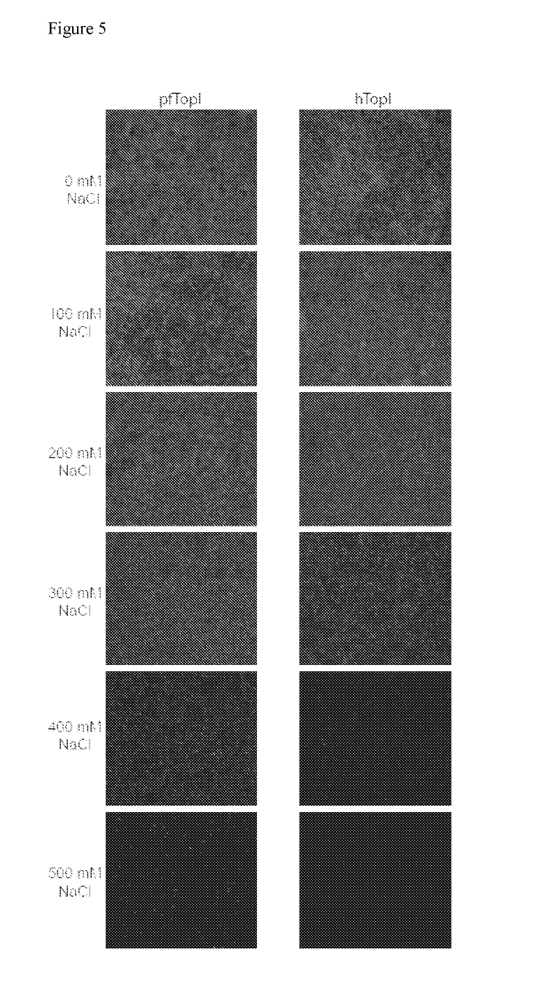

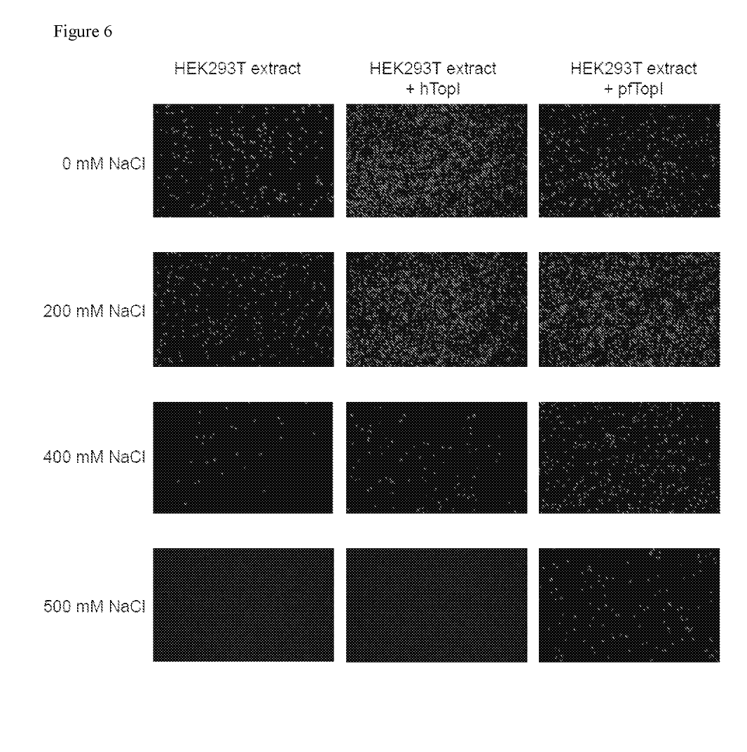

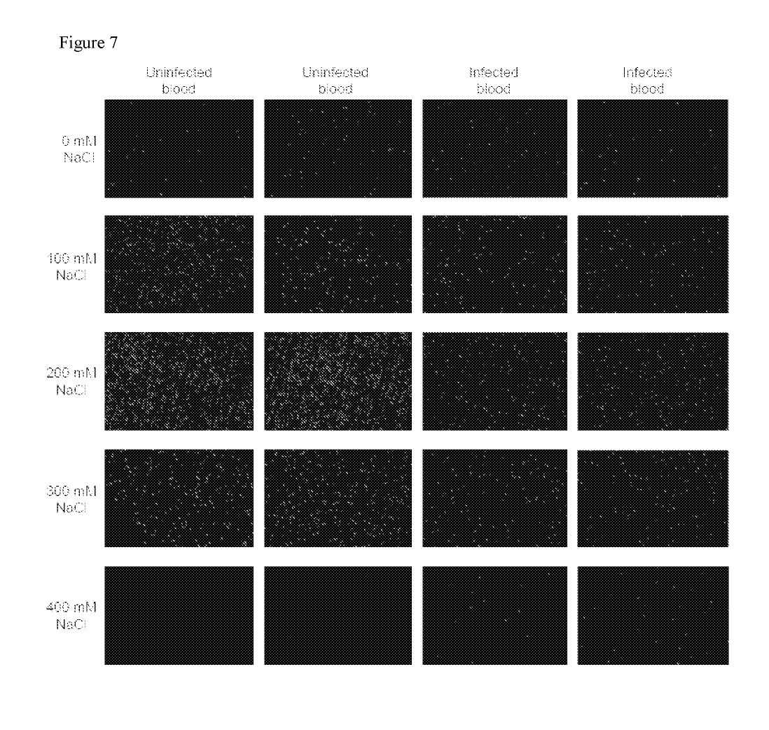

Here, the assay is used for identification of the malaria parasite P. falciparum in crude clinical samples based on the specific detection of single pfTopI cleavage-ligation events. First a synthetic gene encoding pfTopI was cloned and the recombinant protein expressed in and purified from Saccharomyces Cerevisiae to allow characterization of the enzyme. The ability of pfTopI to cleave the classical hexadecameric sequence known as a preferred cleavage site for most other nuclear type IB topoisomerases was investigated using a synthetic 75-mer substrate with this sequence. pfTopI cleaved this substrate between nucleotides −1 and +1, which is the preferred cleavage site for other nuclear type IB topoisomerases, including hTopI, as well as several addition sites located downstream to this position, which is not cleaved by hTopI (FIGS. 1A, and 4). Based on this result it was anticipated that single cleavage-ligation events mediated by pfTopI could be detected in an RCA-based biosensor system using the substrate (Su1) originally developed to detect hTopI activity. As demonstrated in FIG. 5, this expectation held true. Moreover, since pfTopI exhibits a considerably higher salt tolerance than does hTopI (FIG. 5) increasing the salt concentration enabled the specific detection of pfTopI on a background of human cell content including hTopI in extracts from cell lines or human blood (FIG. 6 and FIG. 7). However, the specific detection of pfTopI obtained in this manner was at the cost of sensitivity, with salt (400-500 mM) concentrations high enough to prevent hTopI activity decreasing pfTopI activity.

As shown in FIG. 1A and FIG. 4, in contrast to its human counterpart, pfTopI is able to cleave close to DNA ends with high efficiency. Hence, a DNA substrate which is circularized upon cleavage-ligation close to a DNA end may enable specific detection of pfTopI on a background of the human cell extract without compromising sensitivity of the assay considerably. To address this possibility, purified pfTopI was incubated with each of the substrates Su2-Su6 (FIG. 1B) and the products analysed using the RCA based detection system as schematically outlined in FIG. 1C. The sequence of the substrates Su4, Su5, and Su6 was modified to match the sequence that was cleaved with high efficiency in substrate XX. As evident from FIGS. 1D and E, pfTopI was able to convert Su2-Su6 to closed circles readily detectable in the RCA-based biosensor setup, while hTopI was not (one example shown in FIG. 1D). Of the different substrates and assay conditions tested out the utilization of Su2 appeared the most efficient for specific detection of pfTopI (FIG. 1E).

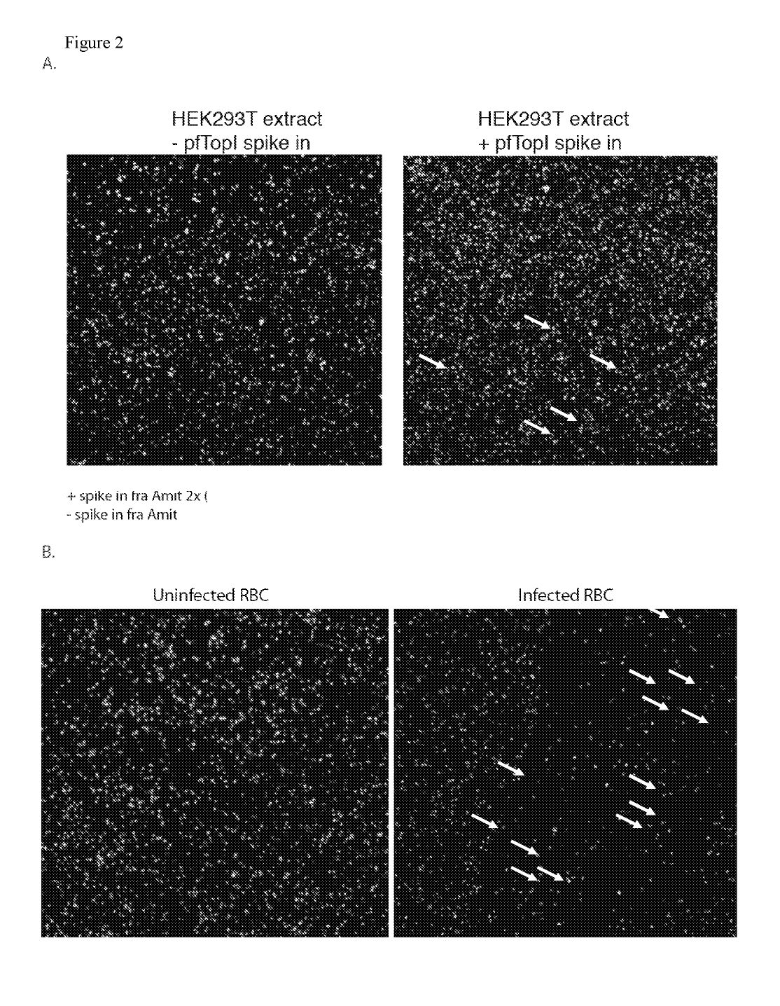

The use of Su2 for detecting pfTopI activity in human cell extracts in the RCA-based biosensor setup was also verified. For this purpose nuclear extracts from HEK-293T cells were incubated with Su1 (which is circularized by hTopI and serve as a control of efficient cell lysis) and Su2 before or after addition of spike-in purified pfTopI followed by RCA and visualization of RCPs as outlined in FIG. 1B. As a control for successful RCA and probe annealing a circularized control circle was added to each sample before annealing to the primer coated slide. As evident from FIG. 2A, red spots corresponding to RCPs originating from Su2 were only observed upon addition of spike-in pfTopI to the extract, verifying that Su2 serves as a substrate specific for pfTopI even in crude cell extracts. As expected, comparative levels of green and blue spots corresponding to RCPs from Su1 and control circles, respectively, could be observed in both samples (FIG. 2A).

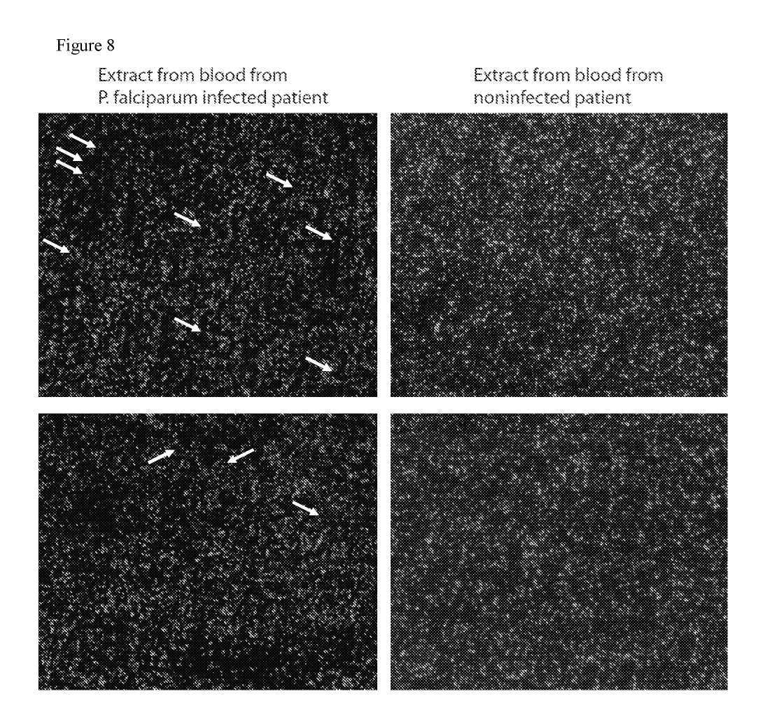

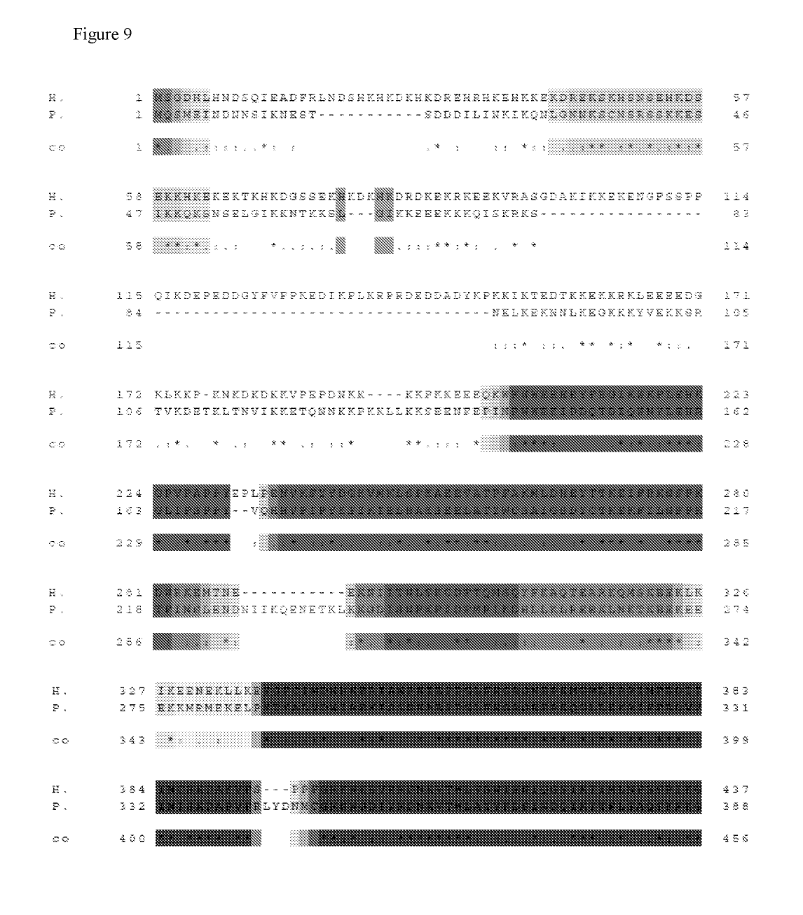

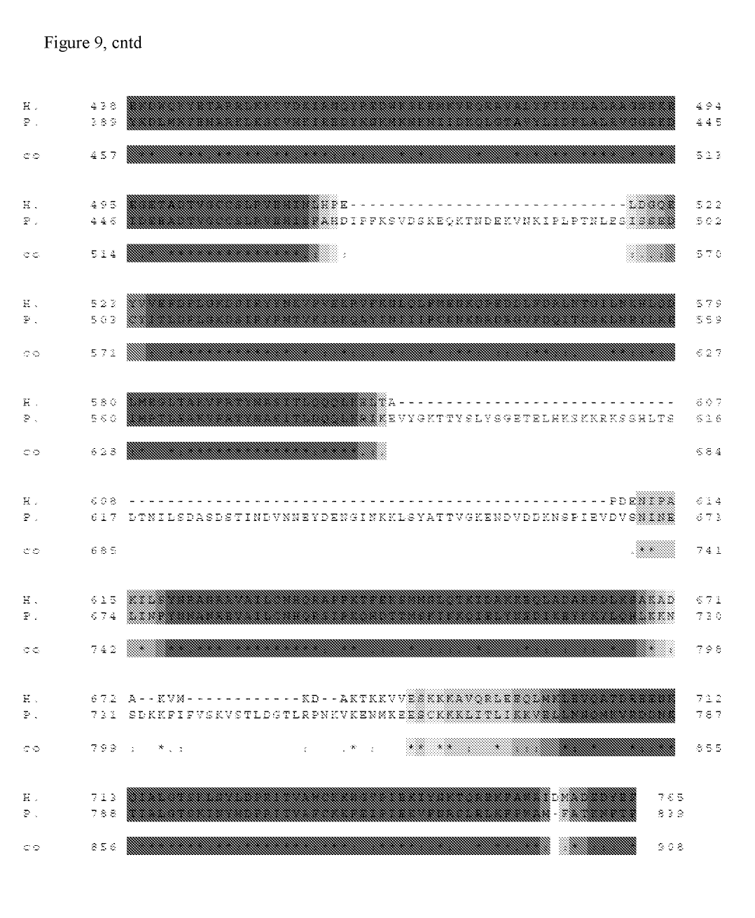

To test the use of pfTopI specific RCA-based detection setup for diagnosis of malaria, extracts from either non-infected or in vitro generated P. falciparum infected human, Red Blood Cells (RBC) were subjected to analysis essentially as described for the experiments depicted in FIG. 2A. Consistent with Su2 being circularized only by pfTopI red signals originating from RCPs of this substrate were observed only after incubation with extracts from P. falciparum infected RBC (FIG. 2B, right panel), whereas signals originating from RCPs of circularized Su1 or the control circle could be observed upon incubation with extracts from both uninfected and infected RBC (FIG. 2B). Note that the hTopI activity observed in extract from infected RBC (green spots in FIG. 2B, left panel) was considerably lower than in extract from noninfected RBC (green spots in FIG. 2B, right panel). Since the same cell extracts were used in both experiments we believe this to be a side effect of cells suffering in different ways from the P. falciparum infection. A similar result as the one shown in FIG. 2B was obtained when analysing extracts from a blood sample from a mildly infected malaria patient (FIG. 8), further verifying the validity of the detection method to specifically detect the presence of P. falciparum parasites in clinical relevant samples

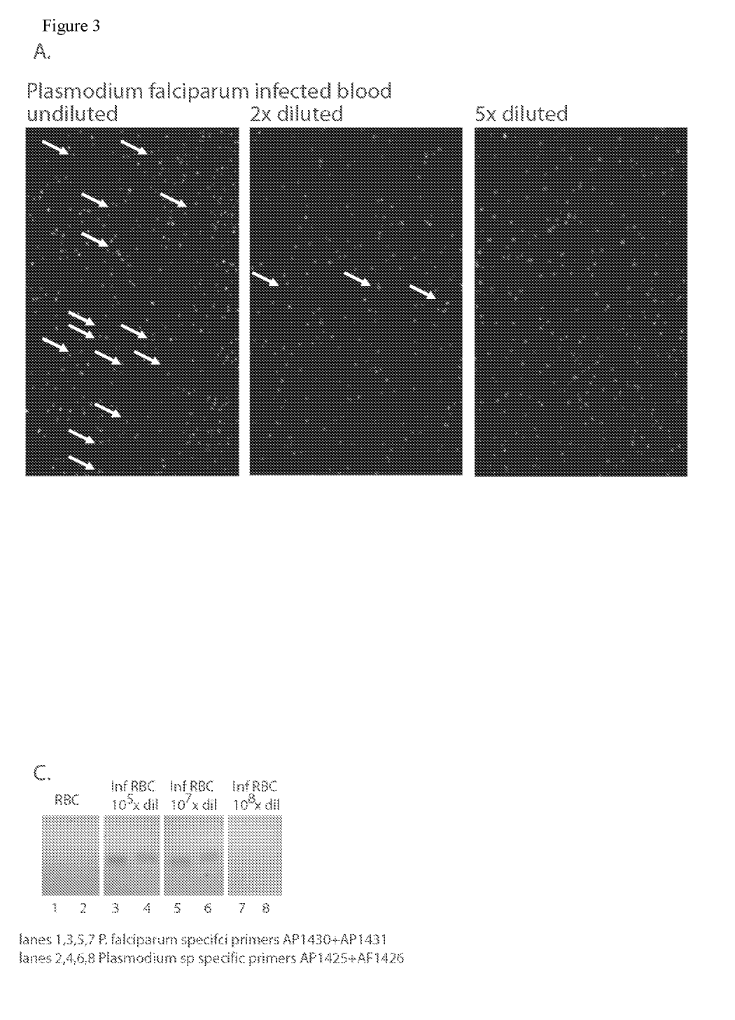

As evident from FIG. 3B the presented method allows the detection of down to 2×10⁴ parasites/μl of RBC. In comparison the detection limit of PCR using standard primers specific for Plasmodium sp. or P. falciparum specific genomic sequences was around 1 parasite/μl of RBC (FIG. 2C), whereas the detection limit of a commercially available malaria RDT was about XX parasites/μl (FIG. 2D). Note, that although PCR is by several orders of magnitude more sensitive than the RCA-based biosensor, at least in its current crude setup, this technique do not allow a quantitative estimation of the infection level (compare lanes 5 and 6 with lanes 3 and 4 of FIG. 3C), which is possible with RCA-based biosensor (compare the right and middle panels of FIG. 3A). Moreover, the PCR analyses required purification and concentration of genomic DNA to perform, whereas the biosensor allowed P. falciparum detection directly in crude cell extracts. Regarding sensitivity the presented RCA-biosensor by far outcompetes current state of the art malaria RDT (compare FIGS. 3A and D).

In conclusion, the present example demonstrates the specific, easy and sensitive detection of malaria in clinical relevant samples by visualizing single cleavage-ligation events mediated by pfTopI. This is achieved by a special developed biosensor system in which each catalytic reaction by pfTopI is converted to a micrometer-sized product readily visible at the single-molecule level. Since each pfTopI, potentially can perform thousands of catalytic reactions without losing activity, the sensitivity of the biosensor is would outcompete current immunohistochemical based diagnostic tools and may allow diagnosis based on non-invasive samples such as mucus or saliva, which typically contain only sparse numbers of P. falciparum parasites. This can be achieved by concentrating the RCP signals. Note, that concentrating RCPs on sequencing beads significantly improve sensitivity of the assay. Wth regard to handling and speed, the present method is superior PCR, and provides a quantitative measurement allowing continuous monitoring of disease development and treatment, which PCR cannot provide.

### Example 2

### Detection of Single Enzymatic Events in Rare- or Single Cells Using Microfluidics

### Methods

Cell Culture and Transfections.

Human embryonic kidney HEK293 cells were cultured in GIBCO's Minimal Essential Medium (MEM) supplemented with 10% fetal bovine serum (FBS) (Atlanta Biologicals), 100 units/mL penicillin and 100 mg/mL streptomycin (Invitrogen) in a humidified incubator (5% CO2/95% air atmosphere at 37° C.). Cells were harvested with 0.25% Trypsin-EDTA (GIBCO) and resuspended in Phosphate-buffered Saline (1×PBS, Cellgro), 1% Pluronic F-68 (Sigma-Aldrich), 0.1% BSA (Invitrogen). The cell densities were adjusted to 0.5-5 million cells/mL and used for enzyme activity detection in the microfluidic system.

Plasmid pCAG-Flpe:GFP for expression of Flpe C-terminally tagged with green fluorescent protein (GFP) in human cells was from Addgene. Transient transfection of pCAG-Flpe:GFP into HEK293 cells was performed using Lipofectamine-2000 (Invitrogen) and 8 μg plasmid DNA and was carried out in GIBCO's Reduced Serum Medium (OPTI-MEM) according to the manufacturer's instructions. 24 h after transfection, cells were harvested with 0.25% Trypsin-EDTA and resuspended in Phosphate-buffered Saline, 1% Pluronic F-68, 0.1% BSA. Transfected cells were mixed with non-transfected cells at the ratios stated in the text and the cell densities adjusted to five million cells/mL (for detection of rare cells) or 0.5 million cells/mL (for addressing the detection limit of the REEAD-microfluidic setup) and used for enzyme activity detection in the microfluidic system or in the “large-volume” bulk experimental setup.

Synthetic DNA Substrates, Probes, and Primers.

Oligonucleotides for construction of the S(TopI), S(Flp), S(Control) substrates, the RCA-primer, and the fluorescently labelled identification probes for the three substrates were purchased from DNA Technology A/S. The sequences of all used the oligonucleotides have been published previously 2.

Rolling Circle Enhanced Enzyme Activity Detection (REEAD) in Bulk Setup.

The single-molecule TopI and Flp activity assays were performed essentially as described by F. F. Andersen, M. Stougaard, H. L. Jorgensen et al., ACS Nano 3 (12), 4043 (2009), except for the preparation of the cell extracts. In brief, mixtures of transfected and non-transfected HEK293 cells (described above) were incubated for 5 min in lysis buffer (10 mM Tris-HCL pH 7.5, 0.5 mM EDTA, 1 mM DTT, 1 mM PMSF, 0.2% Tween 20). Subsequently, S(TopI) and S(Flp) were added to the extract at a final concentration of 100 nM and incubation continued for 30 min at 37° C. RCA-based detection of circularized S(TopI) and S(Flp) in the samples was performed as previously published 2.

Rolling Circle Enhanced Enzyme Activity Detection (REEAD) in Microfluidic System.

The microfluidic setup consists of two devices: a flow-focusing droplet generator and a drop-trap. Both devices were fabricated by conventional soft lithography techniques 13, casting and curing the PDMS prepolymer on a SU-8 3025 (MicroChem) master of a channel height at around 25 μm. PDMS prepolymer (Sylgard 184) was prepared in a 10:1 (base:curing agent) ratio and cured at 65° C. for 1 hr. Prior to the experiments, the channel was wetted with oil/surfactant for at least 15 min. Two syringe pumps (Harvard Apparatus) were used to control the flow rates of oil/surfactant and reagents independently, forming monodisperse water-in-oil droplets at a frequency of 0.8-1.5 kHz. The droplet volume and generation frequency was controlled by the flow rate ratio, determined by the competition between continuous phase (carrier fluid (FC-40 fluorocarbon oil (3M): the oil/surfactant, flow rate 22.5 μL/min) and disperse phase (aqueous reagents: cells, lysis buffer and substrates, flow rate 2.5 μL/min). Cells, prepared as stated above, lysis buffer (10 mM Tris-HCL pH 7.5, 0.5 mM EDTA, 1 mM DTT, 1 mM PMSF, 0.2% Tween 20), and substrates (final concentration of 100 nM in the droplets) were loaded in each their channel in the microfluidic device and droplet generation initiated. The generated droplets were harvested in eppendorf tubes and placed on a primer-printed glass slide (CodeLink Activated Slides from SurModics) prepared as previously described. The PDMS drop-trap was gently placed on top of the glass slide. The geometry of the drop-trap was designed according to the size of generated droplets. The droplets were left to exsiccate for 16 hours. Wash, RCA, and hybridization of probes were performed as previously described 2.

Microscopy. Epifluorescent and bright field images were captured with an inverted fluorescence microscope (Axio Observer, Zeiss). Monocolor emission from each fluorophore was collected and filtered through appropriate filters and dichroics. Image processing and analysis was performed with MetaMorph (v.7.6.5).

### Results

By combining a rolling circle enhanced enzyme activity detection assay with a specially designed microfluidic device, we here demonstrate highly sensitive detection of rare, uncharacteristic cells on a background of bulk wild-type human cells. The combined setup even allowed quantitative detection of enzyme activities in single cells and holds promise for basic research, diagnostic or prognostic purposes.

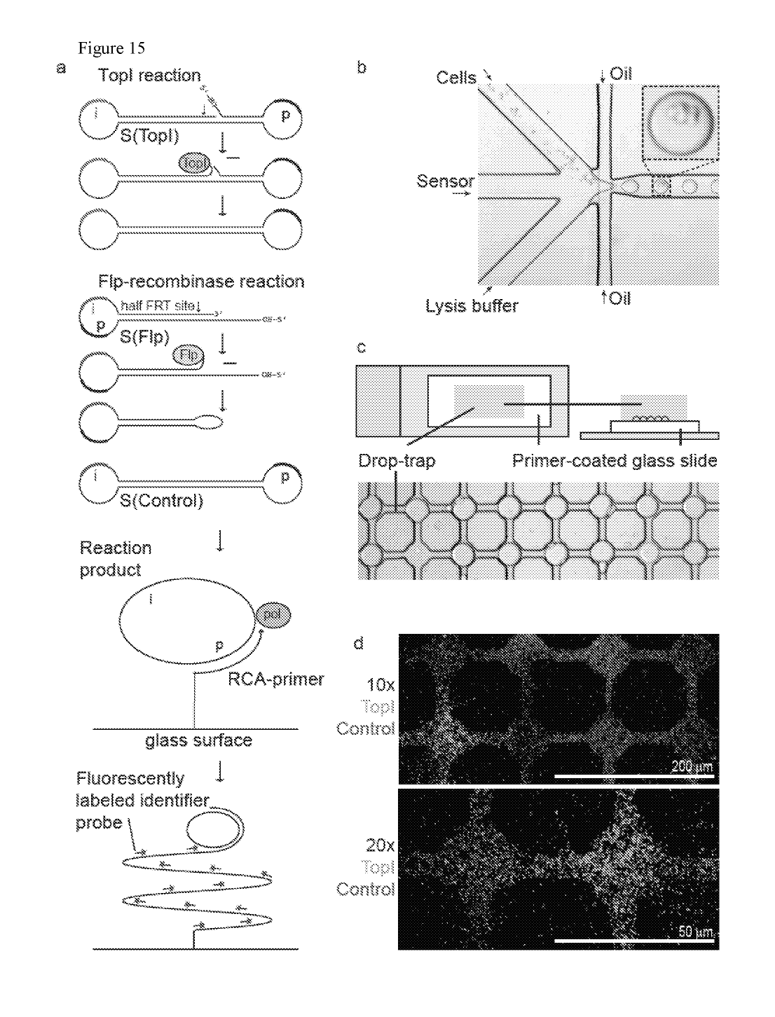

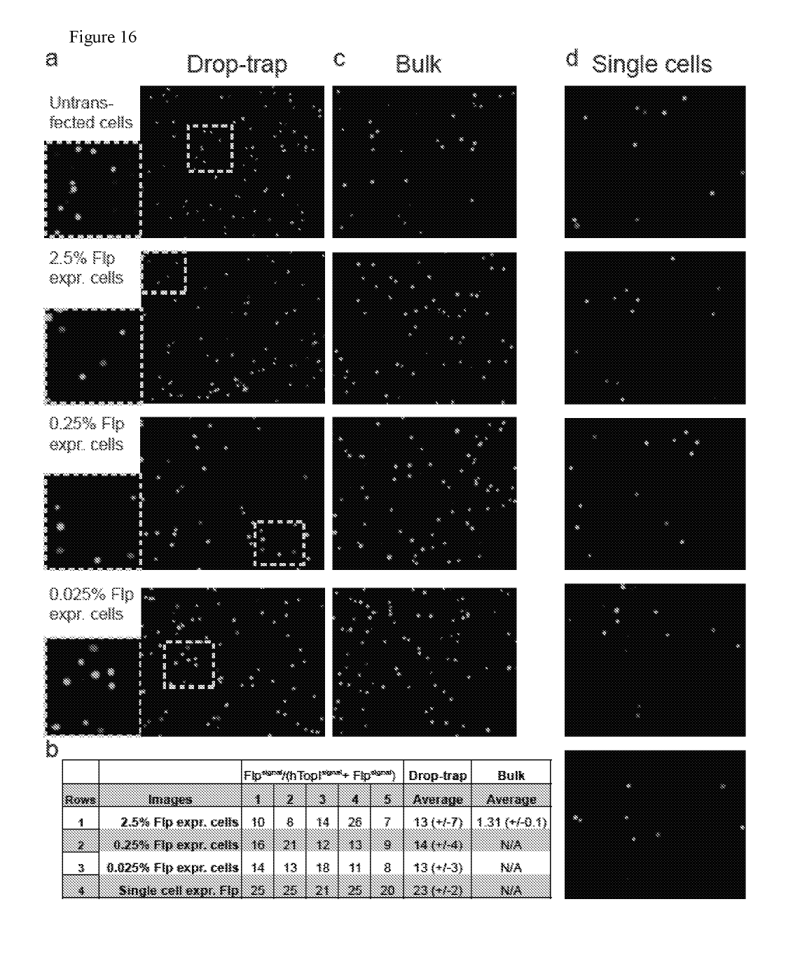

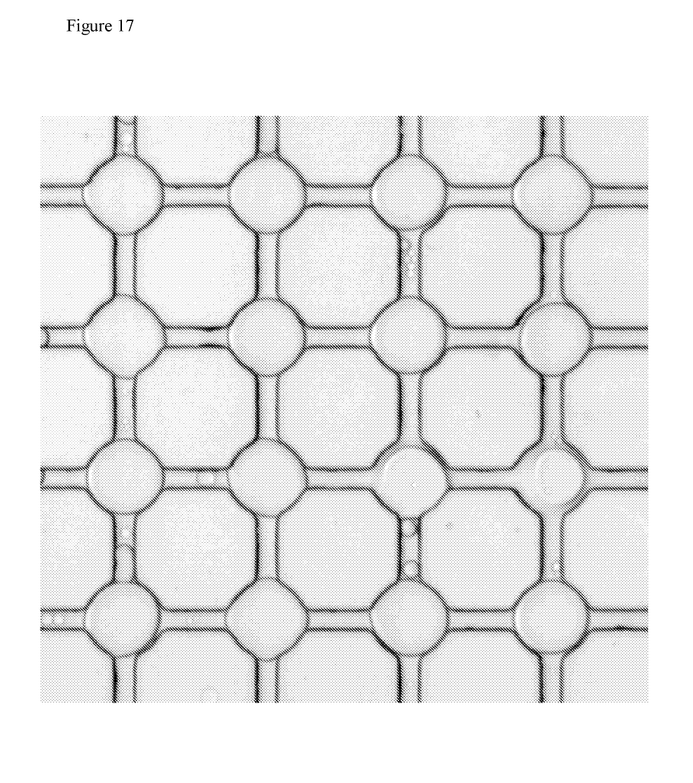

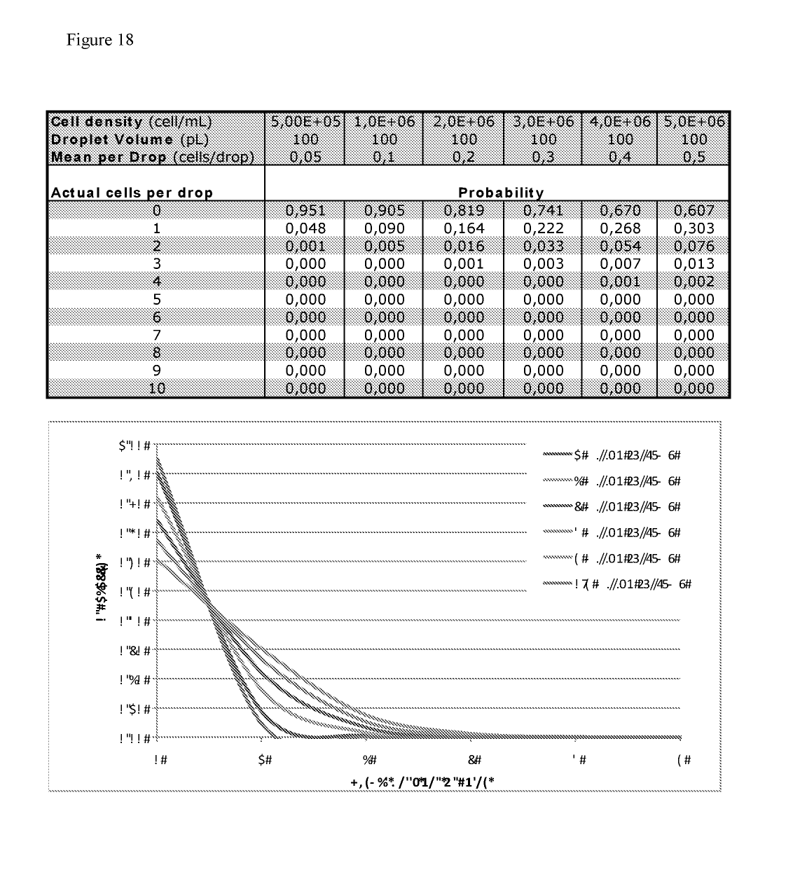

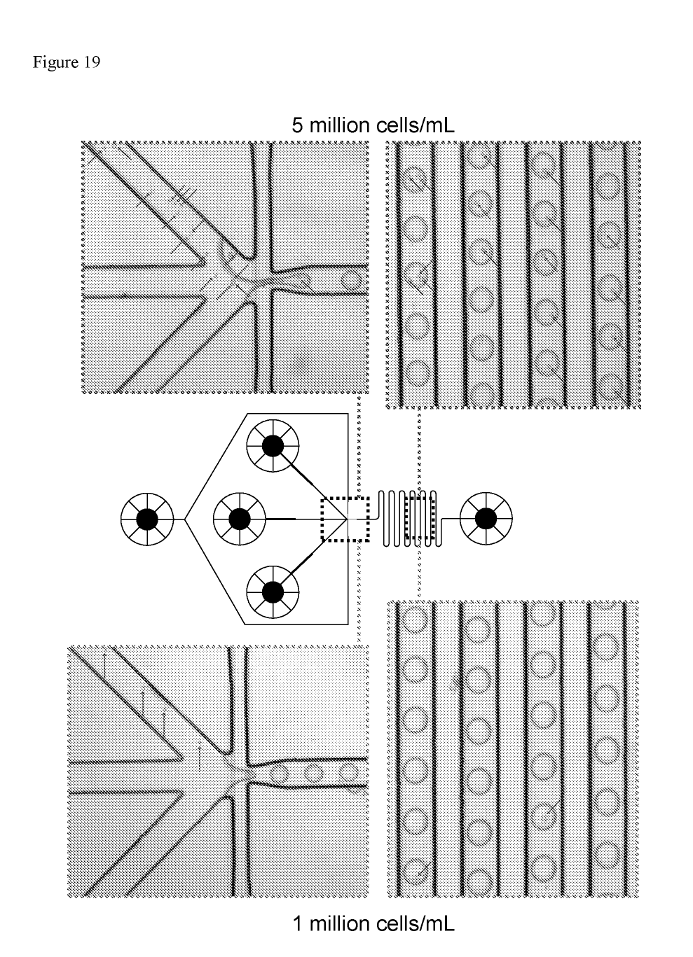

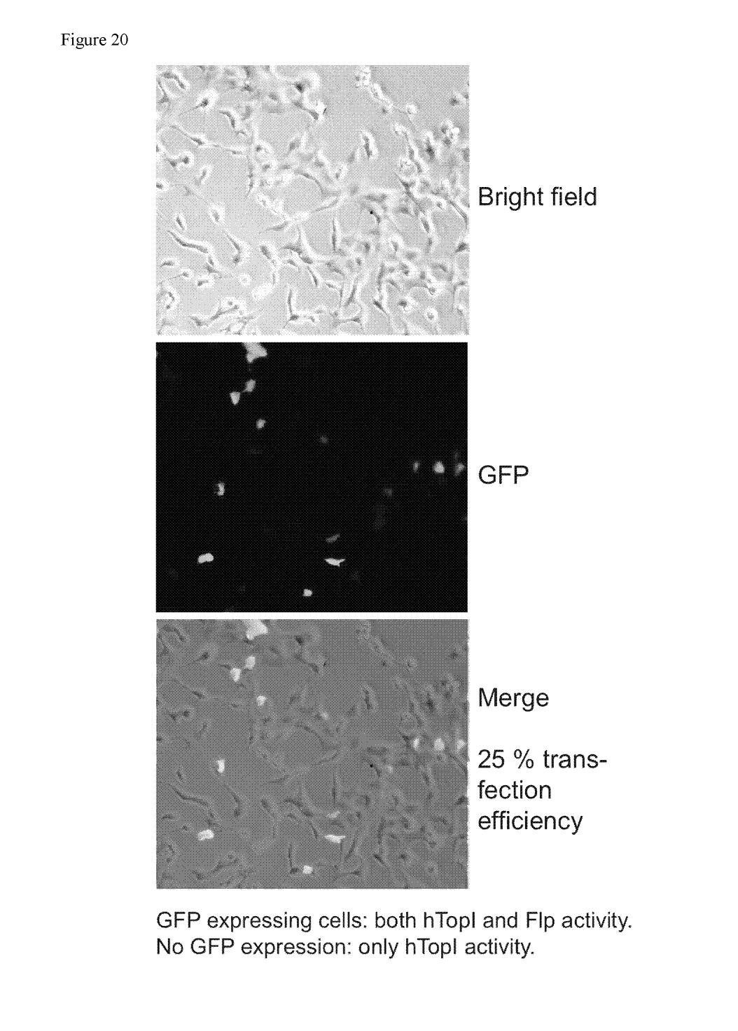

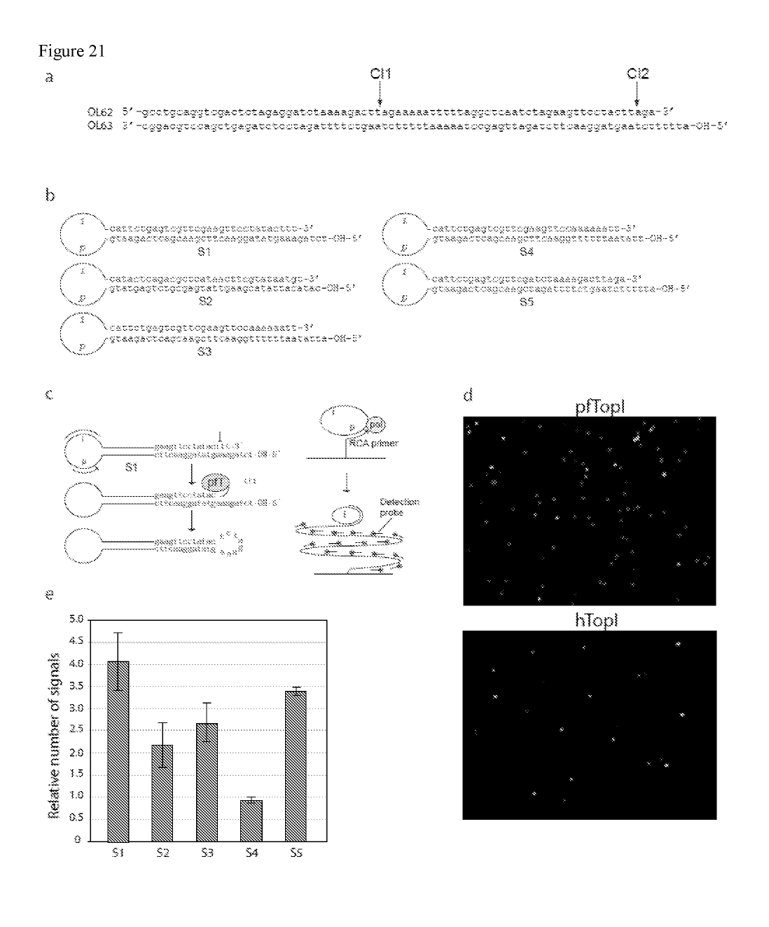

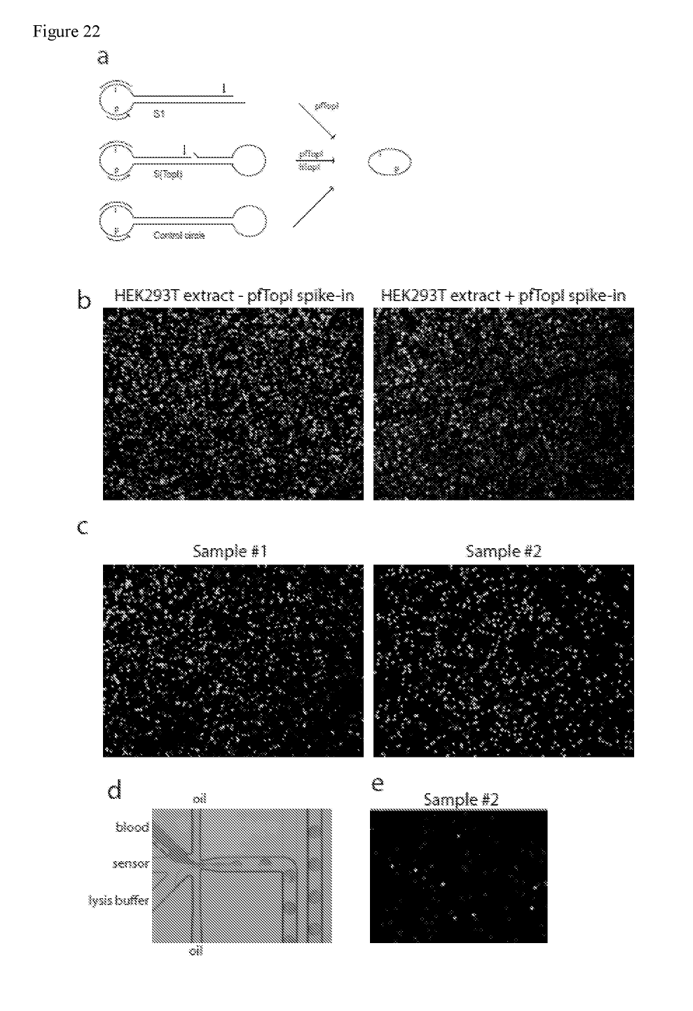

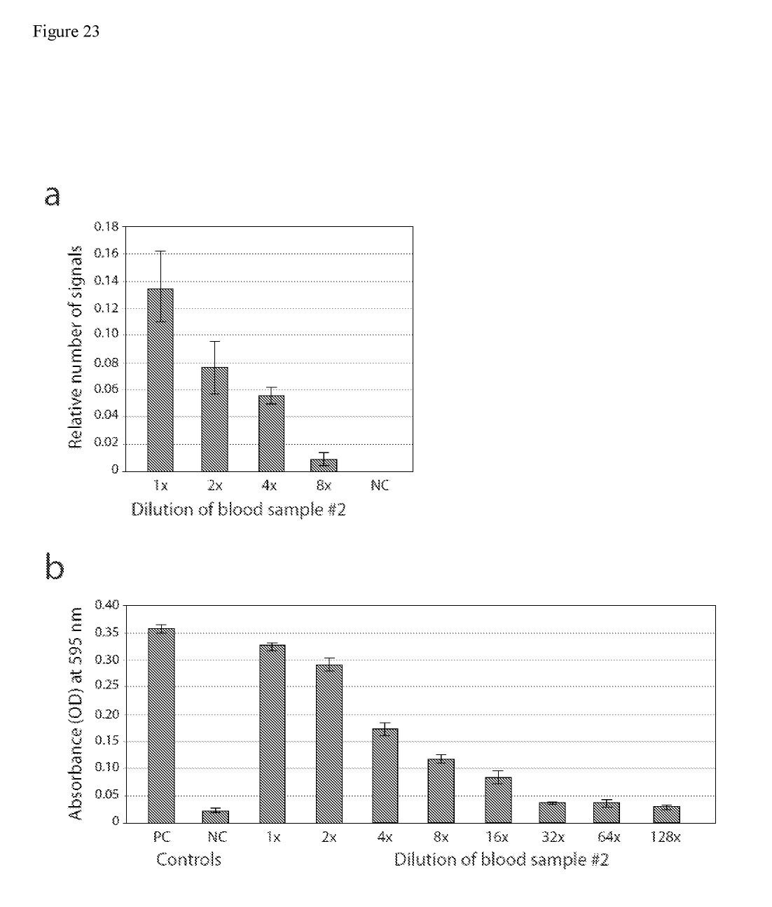

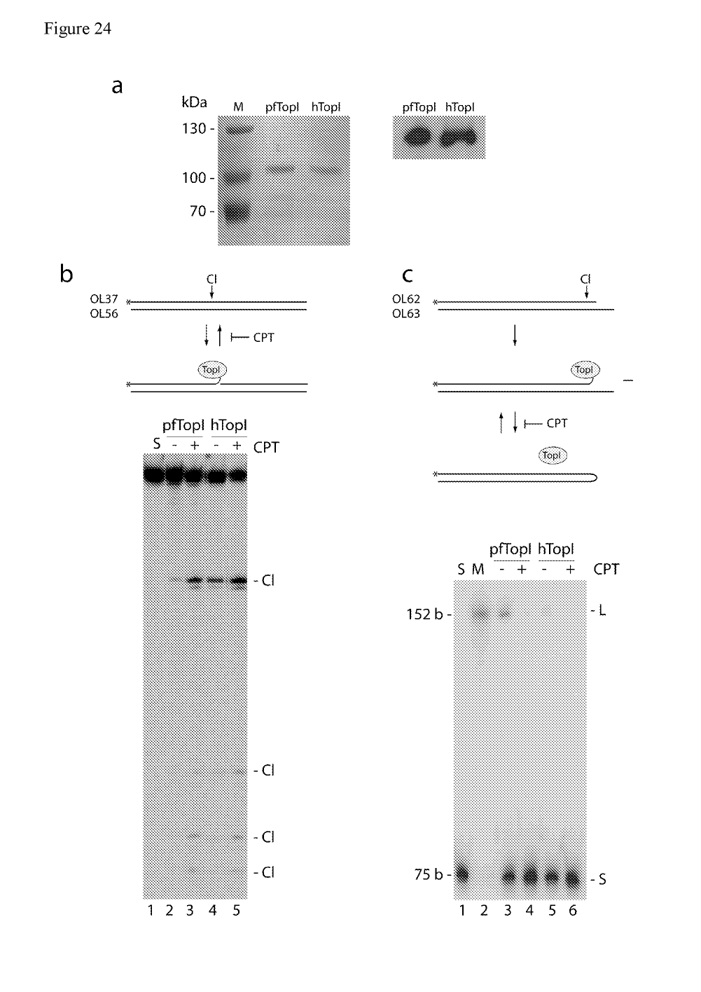

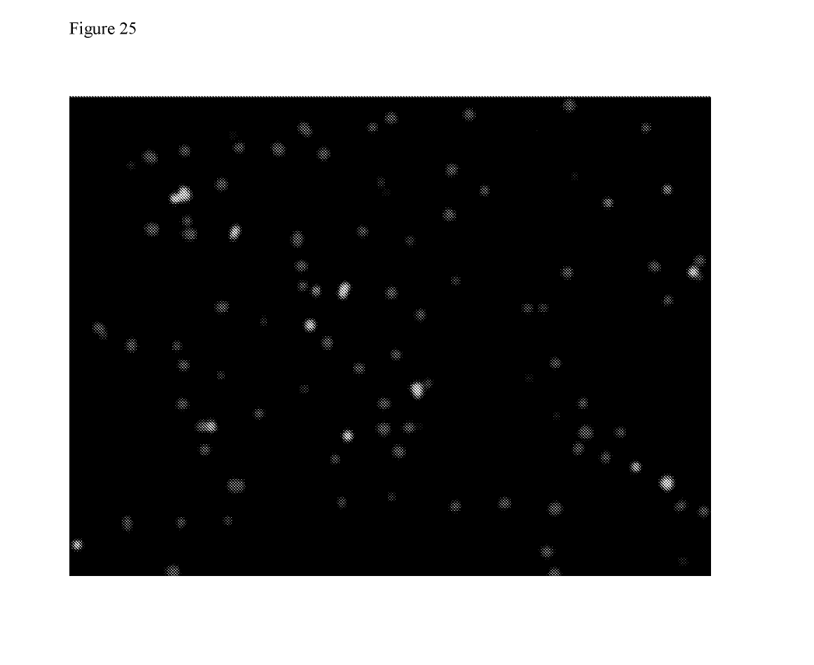

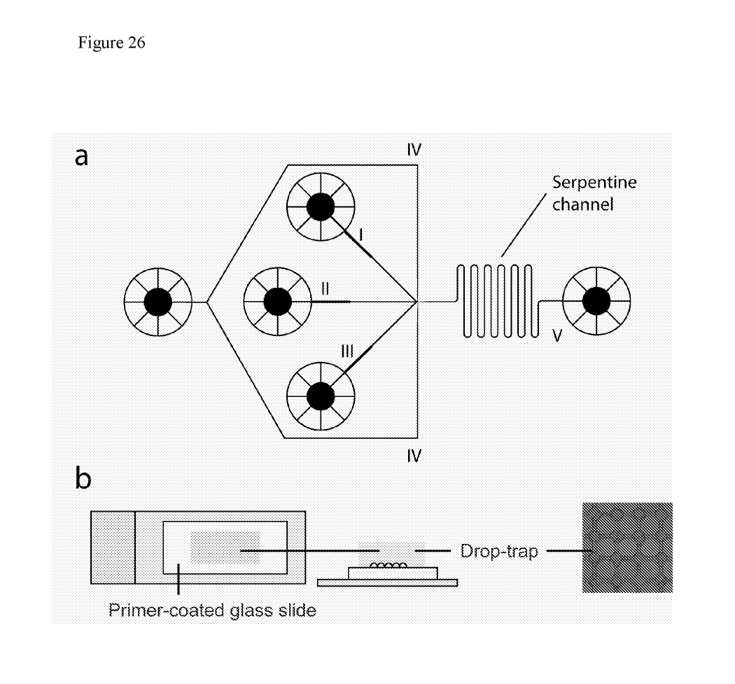

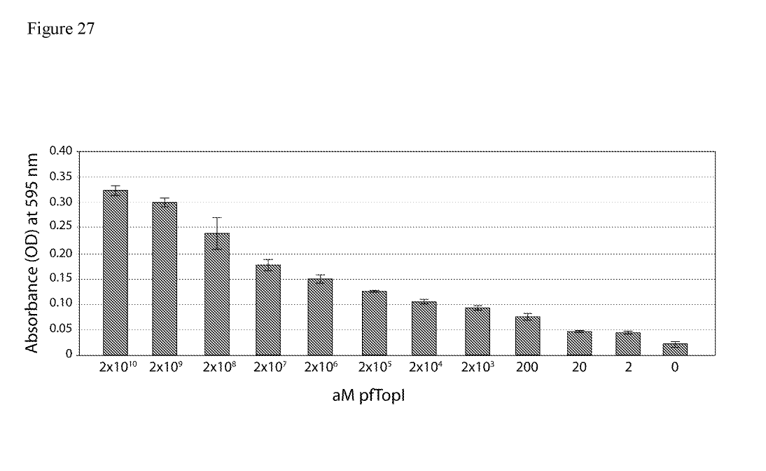

Reliable identification of rare cells different from the bulk of a cell population poses great potential for basic research and for diagnostic or prognostic purposes. The highly sensitive Rolling circle Enhanced Enzyme Activity Detection (REEAD) assay allows analysis of single enzymatic DNA cleavage-ligation events via Rolling Circle Amplification (RCA) of circular DNA products and microscopic visualization of individual Rolling Circle Products (RCP) by hybridization to fluorescent probes (FIG. 15a). In principle, the single-catalytic-event detection limit of REEAD should allow the enzyme content of single cells to be analyzed. However, spreading of signals to a ˜9 mm² area with a handheld pipette hampered sensitivity in the original “large-volume” bulk setup. Here, we present the integration of REEAD with a microfluidic setup, allowing the enzymatic content of one or few cells to react with DNA substrates within a minimalized volume and the subsequent concentration of signals to small cavities of a drop-trap device. A concentration independent detection of rare Flp-recombinase expressing human cells is demonstrated on a background of wild-type cells and multiplexed detection of Flp-recombinase and hTopI activities in single cells. The substrates S(TopI) or S(Flp) for hTopI or Flp-recombinase REEAD were:

S(Topl),
(SEQ ID NO: 25)
5′-AGAAAAATTT TTAAAAAAAC TGTGAAGATC GCTTATTTTT
TTAAAAATTT TTCTAAGTCT TTTAGATCCC TCAATGCTGC
TGCTGTACTA CGATCTAAAA GACTTAGA-3′;
S(Flp),
(SEQ ID NO: 26)
5′-TCTAGAAAGT ATAGGAACTT CGAACGACTC AGAATGAGGC
TCAATCTAAT GGACCCTCAA TGCACATGTT TGGCTCCCAT
TCTGAGTCGT TCGAAGTTCC TATACTTT-3′.

Each of the substrates comprised one oligonucleotide that is converted to a closed circle by a single hTopI or Flp-recombinase cleavage-ligation event. As a positive control of RCA, a pre-formed DNA circle was used (S(control)) (FIG. 15a). To investigate whether REEAD could be integrated with the microfluidic setup (FIG. 15b) HEK293 cells, to be analyzed for endogenous hTopI activity, were loaded into one channel, S(TopI) and S(control) into a second, and lysis buffer into a third channel of the microfluidic device. By competition with oil the four components were confined in lipid surrounded picoliter droplets, which were directed through a serpentine channel to ensure complete content mixing (FIG. 15b). Cell lysis allowed hTopI to interact with and circularize S(TopI). After exit from the microfluidic system, single droplets were captured in each their cavity of the drop-trap (FIGS. 15c and 17) and exsiccated on a DNA primer-coated glass slide. This allowed RCA of S(control) and circularized S(TopI). RCA of unreacted S(TopI) was prevented as described by M. Stougaard, J. S. Lohmann, A. Mancino et al., ACS Nano 3 (1), 223 (2009). The resulting RCPs were visualized at the single-molecule level by microscopy upon annealing of fluorescent probes. As shown in FIG. 15d, the combination of REEAD and microfluidics enabled multiplexed detection of S(control) (blue) and hTopI reacted S(TopI) (green) in a pattern matching the drop-trap cavities. In the presented experiment the microfluidic system was fed with five million cells/mL. As estimated from the Poisson distribution (FIG. 18) and confirmed experimentally (FIG. 19) this cell density resulted in −60% of droplets without cells and −40% with one or more cells 8. Consistently, all drop-trap cavities contained equally distributed S(control) originating blue signals, while only a part of them contained green signals arising from circularized S(TopI).

To investigate how the combined REEAD-microfluidic setup performs in detecting rare cells different from the bulk of a cell population, we used HEK293 cells containing different proportions of Flp-recombinase expressing cells as a model (FIG. 20). Five million cells/mL containing 2.5%, 0.25% or 0.025% Flp-recombinase expressing cells were loaded into the microfluidic device together with S(TopI), S(Flp) and lysis buffer as described above. After entrapment of droplets and RCA, circularized S(TopI) was visualized by green and circularized S(Flp) by red fluorescence. As evident from FIG. 16a, red Flp-recombinase specific signals (dark spots) could be detected on the background of green signals (light spots) originating from endogenous hTopI activity present in all the cells. Moreover, although the number of drop-trap cavities containing red signals decreased with decreasing density of Flp-recombinase expressing cells the average percentage of Flp-recombinase specific red signals (dark spots) in the drop-trap cavities that did contain red signals was similar regardless the dilution of Flp-recombinase expressing cells (FIG. 16b). Note that, as discussed below, the relatively large deviation of red (dark spots) signals present in individual drop-trap cavities most probably results from the uptake of more than one cell in some droplets when feeding the system five million cells/mL (FIGS. 18 and 19). In comparison to the results obtained by microfluidic-combined REEAD, red signals (dark spots) originating from Flp-recombinase activity was not detectable in cell populations containing less than 2.5% Flp-recombinase expressing cells when measured in a “large-volume” bulk experimental setup (FIG. 16c).

To address the detection limit of the REEAD-microfluidic setup, 0.5 million cells/mL containing 2.5% Flp-recombinase expressing cells were loaded into the system and the activity of Flp-recombinase or hTopI detected. At this cell density no more than one cell was encapsulated in each droplet (FIGS. 18 and 19) and, hence, the signals in each drop-trap cavity (FIG. 17 and FIG. 16d) represented the enzyme activities of a single cell. The figure shows the result of encapsulating Flp-recombinase expressing cells. However, cavities with green signals only, representing a cell without Flp-recombinase, were also observed. The percentage of Flp-recombinase originating signals (red/dark spots) relative to all signals in single cells varied between 20-25% with an average of 23+/−2% (FIG. 16b). When comparing this to the results obtained with five million cells/mL it is clear that when using the high cell density the amount of cells and the relative distribution of wild-type versus Flp-recombinase expressing cells trapped in each droplet varies. For example image #4 in row 1 of FIG. 16b may result from entrapment of one or more Flp-recombinase expressing cells while images #3 and #5 in the same row may result from encapsulation of Flp-recombinase expressing and wild-type cells in the ratio 1:2 and 1:3, respectively.

In conclusion, the detection of Flp-recombinase originating signals independent of the density of Flp-recombinase expressing cells in a population taken together with the comprehensive detection of signals from hTopI or Flp-recombinase activity in single cells demonstrates that the REEAD-microfluidic setup allow diminutive numbers of uncharacteristic cells in a population to be discovered. The high sensitivity of the REEAD-microfluidic setup compared to the conventional “large-volume” bulk setup without doubt relies on the diminished reaction volume and subsequent concentration of signals. These features of the REEAD-microfluidic setup hold promise for analysis of cell populations including cell-to-cell variations for research purposes and for early diagnosis/prognosis of cancer or pathogen infections. Indeed, existing RCA-based single molecule techniques for detection of disease relevant nucleotide sequences or proteins can be combined with the microfluidic setup. In particular, the REEAD-microfluidic setup can be used for the identifying type I topoisomerase-expressing microorganisms, such as Plasmodium falciparum and/or Mycobacterium tuberculosis. In this way, the REEAD-microfluidic setup may be used for the diagnosis of malaria and tuberculosis, respectively.

### Example 3

### Plasmodium Topoisomerase I Specific Nucleotide Biosensor for Diagnosis of Malaria

### Methods

SDS PAGE and Western blotting: pfTopI and hTopI were analyzed by electrophoresis on 10% SDS polyacrylamide gels and the proteins either stained with Coomassie brilliant blue following standard procedures or transferred to a nitrocellulose membrane in 25 mM Tris, 192 mM glycine, 0.1% (w/v) SDS and 20% methanol. Western blotting was performed using standard procedures (primary antibody, polyclonal antibody to hTopI from Scleroderma Patient Serum (TopoGEN); secondary antibody, ImmunoPure Goat Anti-Human IgG-HRP (Thermo Scientific)).

Synthetic substrates for cleavage assays: All oligonucleotides were purchased from DNA Technology A/S and purified by denaturing polyacrylamide gel electrophoresis. The sequences of the oligonucleotides are as follows: OL37: 5′-CGAATTCGCT ATAATTCATA TGATAGCGGA TCCAAAAAAG ACTTAGAAAA AAAAAAAGCT TAAGCAA26, OL56: 5′-TTGCTTAAGC TTTTTTTTTT TCTAAGTCTT TTTTGGATCC GCTATCATAT GAATTATAGC GAATTCG26, OL62: 5′-GCCTGCAGGT CGACTCTAGA GGATCTAAAA GACTTAGAAA AATTTTTAGG CTCAATCTAG AAGTTCCTAC TTAGA, OL63: 5′-ATTTTTCTAA GTAGGAACTT CTAGATTGAG CCTAAAAATT TTTCTAAGTC TTTTAGATCC TCTAGAGTCG ACCTGCAGGC. The oligonucleotides representing the scissile strands (OL37 and OL62) were 5′-radiolabeled by T4 polynucleotide kinase (NEB) using [γ-32P]ATP as the phosphoryl donor. The oligonucleotides were annealed pairwise with a 2-fold molar excess of the bottom strand over scissile strand as previously described.

Detection of PfTopI activity using radio-labeled DNA substrates: DNA cleavage reactions were carried out by incubating 20 nM duplex OL37/OL56 or OL62/OL63 with 500 fmol of pfTopI or hTopI in the absence or presence of 60 μM CPT for 20 min at 37° C. in 10 mM Tris (pH 7.5), 5 mM MgCl2, and 5 mM CaCl2 in a final volume of 20 μl. After the 20 min incubation, reactions were stopped with 0.5% (w/v) SDS. Samples were subjected to ethanol precipitation, resuspended in 10 μl of 1 mg/ml trypsin and incubated at 37° C. for 30 min. Reaction products were analyzed by denaturing polyacrylamide gel electrophoresis following standard procedures, and radioactive bands were visualized by PhosphorImaging.

### Sequences of Oligonucleotide Sensor, Primers and Probes

S(hTopI): 5′-AGAAAAATTT TTAAAAAAAC TGTGAAGATC GCTTATTTTT TTAAAAATTT TTCTAAGTCT TTTAGATCCC TCAATGCTGC TGCTGTACTA CGATCTAAAA GACTTAGA1.

pfTopI(S1): 5′-TCTAGAAAGT ATAGGAACTT CGAACGACTC AGAATGACTG TGAAGATCGC TTATCCTCA ATGCACATGT TTGGCTCCCA TTCTGAGTCG TTCGAAGTTC CTATACTTT7.

pfTopI(S2): 5′-CATACATTAT ACGAAGTTAT GAGCGTCTGA GTATGACTGT GAAGATCGCT TATCAGTGAA TGCGAGTCCG TCTACTCATA CTCAGACGCT CATAACTTCG TATAATGT7.

pfTopI(S3): 5′-ATTATAATTT TTTGGAACTT CGAACGACTC AGAATGACTG TGAAGATCGC TTATCCTCAA TGCACATGTT TGGCTCCCAT TCTGAGTCGT TCGAAGTTCC AAAAAATT.

pfTopI(S4): 5′-TTATAATTTT TTGGAACTTC GAACGACTCA GAATGACTGT GAAGATCGCT TATCCTCAAT GCACATGTTT GGCTCCCATT CTGAGTCGTT CGAAGTTCCA AAAAATT.

pfTopI(S5): 5′-ATTTTTCTAA GTCTTTTAGA TCGAACGACT CAGAATGACT GTGAAGATCG CTTATCCTCA ATGCACATGT TTGGCTCCCA TTCTGAGTCG TTCGATCTAA AAGACTTAGA.

Control-circle substrate: 5′-AGAAAAATTT TTAAAAAAAC TGTGAAGATC GCTTATTTTT TTAAAAATTT TTCTAAGTCT TTTAGATCCCGA GATGTACCGC TATCGTCATG ATCTAAAAGA CTT. Control-circle was prepared as described previouslyl.

RCA primer: 5′-AMINE-CCAACCAACC AACCAAATAA GCGATCTTCA CAGT1.

### Fluorescent Probes:

For detection of S(hTopI): 5′-“F”-GTAGTACAGC AGCAGCATTG AGG1.

For detection of S1-S5: 5′-“F”-GGAGCCAAAC ATGTGCATTG AGG7.

For detection of control-circle: 5′-“F”-CCGAGAT GTACCGCTAT CGT.

“F” indicates fluorescent labelling where Cy5, rhodamine or FITC were used for blue, red or green fluorescence, respectively.

### Results and Discussion

Like human topoisomerasel (hTopI), pfTopI belongs to the family of nuclear type IB topoisomerases, which introduce transient single-strand breaks in double-stranded DNA with preference for a very degenerate consensus sequence. Cleavage results in a covalent enzyme-DNA intermediate allowing religation of the generated nick (FIG. 23).

To demonstrate that different DNA recognition by pfTopI and hTopI allows the design of a pfTopI-specific biosensor to be used in a Rolling-Circle-Enhanced-Enzyme-Detection (REEAD) setup, purified recombinant pfTopI or hTopI were reacted with double-stranded DNA in a standard cleavage assay (FIG. 24). The result demonstrated that besides cleaving the sites cleaved by hTopI, pfTopI can cleave DNA close to a 3′-end and ligate a protruding 5′-end of the non-scissile strand, which hTopI cannot (FIG. 21a). Based on this, five different oligonucleotides with potential of being circularized specifically by pfTopI cleavage-ligation were designed. These oligonucleotides (PfTop1(S1-S5)) all folded into a hairpin structure containing a probe- and a primer-annealing sequence in the single-stranded loop and a potential pfTopI recognizable sequence at the end of the double-stranded stem region (FIG. 1b). The ability of PfTop1 (S1)-(S5) to be circularized by pfTopI or hTopI was tested in the REEAD setup (FIG. 21c) by incubation one at a time with each of the purified enzymes, followed by solid-support RCA of closed circles as previously described by Stougaard, M. et al. ACS Nano 3, 223-233 (2009). RCPs were visualized microscopically at the single-molecule level upon hybridization of red-fluorescent probes. To allow comparative quantification of signals generated in different reactions, a known concentration of control-circle with a unique probe-annealing sequence was added to the reaction mixtures before RCA and resulting RCPs visualized using a green-fluorescent probe. Estimating the circularization efficiency of pfTopI(S1)-(S5) by pfTopI in terms of frequency of red signals relative to green signals demonstrated pfTopI(S1) to be the most efficient sensor of pfTopI (FIGS. 21d and e). Hence, pfTopI(S1) was chosen for the following experiments. None of the oligonucleotides were circularized by hTopI (FIG. 21d, and data not shown).

The specificity of pfTopI(S1) for pfTopI in crude biological samples was addressed using nuclear extract from human HEK293T cells with or without spike-in pfTopI as a model for Plasmodium infection. Besides pfTopI(S1), S(TopI), previously demonstrated to sense specifically hTopI in crude cell extracts, and control-circle was added to the reaction mixtures as positive controls for nuclear extraction and RCA/probe hybridization, respectively (FIG. 22a). Red/dark signals corresponding to single RCPs matching circularized pfTopI(S1) was observed only upon addition of pfTopI spike-in, whereas green and blue signals originating from circularized S(TopI) and control-circle, respectively, were observed in both samples (FIG. 22b). This demonstrates the specificity of pfTopI(S1) for pfTopI even on a background of human nucleus content. Note, due to characteristics shared between hTopI and pfTopI, the latter enzyme circularizes S(TopI) (FIG. 25) as well as pfTopI(S1). Consequently, green signals observed in FIG. 22b, right panel, originated from hTopI and pfTopI activity in combination where pfTopI(S1) and S(TopI) competed for reaction by pfTopI at the expense of sensitivity.

To address the performance of pfTopI-specific REEAD in sensing Plasmodium in clinically relevant samples, pfTopI(S1) and S(TopI) were reacted with extracts prepared from blood samples from an uninfected (sample #1) or a P. falciparum-infected patient (sample #2). Control-circle was added to the reaction mixtures as a positive control. Color codes were the same as in FIG. 22b. As evident from FIG. 22c, red/dark pfTopI-specific signals were observed only upon incubation of the REEAD sensors with extracts from sample #2, while green and blue signals were observed after incubation with both extracts. Sample #2 originated from a pauci-parasitic patient with a parasitemia below 0.0001-0.0004% representing the detection limit of traditional microscopy-based diagnosis, although detectable by PCR (data not shown). Hence, even in the presented crude setup, the REEAD assay performed better than state-of-the-art diagnostic assays with regard to sensitivity. Testing samples from several uninfected or pauci-parasitic patients confirmed generality of the results shown in FIG. 22c (data not shown).

The need for extensive sample preparation poses an obstacle for the practical use of diagnostic tests. In the experiments shown in FIG. 22c, extracts were prepared from 10 mL of blood in a procedure involving several centrifugations. To investigate if such preparation could be avoided, REEAD was combined with a simple microfluidic channel lab-on-a-chip device (Cho, E. J., Yang, L., Levy, M. & Ellington, A. D. Using a deoxyribozyme ligase and rolling circle amplification to detect a non-nucleic acid analyte, ATP. J Am Chem Soc 127, 2022-2023 (2005)) allowing confinement of infected blood cells, biosensors, control-circle and low-salt lysis buffer in pL droplets in which the reaction took place (FIG. 22d and FIG. 26). Subsequently, droplets were retained in a drop-trap and exsiccated on a primer-coated glass slide to support RCA and visualization of RCPs. Using this integrated setup is was possible to detect P. falciparum infection using only 200 μL of completely unprocessed blood sample #2 (FIG. 22e).

To investigate if the Plasmodium-specific REEAD could be adapted to simple colorimetric readout, suitable for low-resource settings without compromising sensitivity, an additional enzymatic step was introduced to the assay by coupling streptavidin-fused HRP to biotinylated RCPs as described by Yan, J. et al. An on-nanoparticle rolling-circle amplification platform for ultrasensitive protein detection in biological fluids. Small 6, 2520-2525 (2010). HRP oxidizes colorless tetramethylbenzidine to a blue-colored form, detectable to the naked eye or by spectrophotometric measurements. This, of course, is at the expense of the possibility of multiplexing since RCPs originating from different circles cannot be distinguished. Hence, S(TopI) and control-circles used as internal controls for microscopic readout had to be omitted and replaced with separate control experiments for the colorimetric readout. This, however, imposed the advantage of preventing competition between S(TopI) and pfTopI(S1) for pfTopI, as discussed above. The two readout formats were compared by reacting pfTopI(S1) with increasing dilutions of extracts from blood sample #2 followed by microscopic visualization of RCPs or by spectrophotometric measurement of HRP substrate conversion. As evident from FIG. 23 colorimetric readout increased sensitivity of REEAD by a factor two compared to microscopic readout. When using purified pfTopI to circularize S1, the HRP reaction allowed direct visual detection of 200 aM of pfTopI (data not shown) whereas spectrophotometric measurement allowed detection of 2 aM pfTopI (FIG. 27).

In conclusion the pfTopI-specific REEAD setup presented here allowed the highly sensitive detection of Plasmodium infection in even small volumes of unprocessed blood samples. The presented assay out-competes current state-of-the-art malaria diagnostic assays with regard to sensitivity, time-of-performance and ease of the procedure. Thus, REEAD can form the basis for novel user-friendly and low-cost kits for first-line detection of malaria, which may be of particular importance in low-resource settings. Compared to most published RCA-based systems for detection of nucleotide sequences or non-reactive proteins, the specific detection of an enzyme activity presents the advantage of being suitable for solution detection, requiring little sample preparation and including an inherent initial enzymatic amplification step. The presented Plasmodium-specific REEAD is an important proof-of-principle for the usability of enzyme-specific biomarkers in diagnostics. Moreover, pfTopI is a potential target for new drugs in the combat against multi-drug resistant malaria, and therefore, the presented REEAD provide an important mean for fast high-throughput drug screening setups in a method for drug discovery according to the present invention.

### Methods

Nucleotide Sensors, Primers and Probes:

All oligonucleotides were purchased from DNA Technology A/S. The sequences of the oligonucleotides are shown in Supplementary Information.

Enzyme Expression and Purification:

The pfTopI gene (PlasmoDB accession number PFE0520c)²³ was codon optimized (by GeneArt) for expression in Saccharomyces cerevisiae. The optimized gene was PCR amplified and cloned into the pYES2.1/V5-His-TOPO vector (Invitrogen). A positives clone was identified by sequencing and the plasmid pPFT100 was transformed into the yeast S. cerevisiae top1Δ strain RS190 (a kind gift from R. Sternglanz, State University of New York, USA) according to standard procedures. pfTopI was expressed and purified as previously described for human topoisomerasel²⁴. hTopI was expressed and purified as previously described²⁴. The protein concentrations were estimated from Coomassie blue-stained SDS-polyacrylamide gels by comparison to serial dilutions of BSA.

Cell Culture and Nuclear Extract Preparation:

Human embryonic kidney HEK293T cells were cultured in Dulbecco's Modified Eagle Medium (GIBCO) supplemented with 10% fetal bovine serum (FBS) (GIBCO), 100 units/mL penicillin and 100 mg/mL streptomycin (Invitrogen). Cells were incubated in a humidified incubator (5% CO₂/95% air atmosphere at 37° C.). Cells were harvested with 0.5% Trypsin-EDTA (GIBCO). Media was discarded and the cell washed in Phosphate-Buffered Saline (1×PBS) prior to nuclear extraction performed as previously described⁷. The cell extracts were used for REEAD directly or spiked with purified pfTopI prior to REEAD.

Preparation of Extracts from Blood Samples:

30 mL RBC lysis buffer (Gentra Puregene) was added to 10 mL of blood (uninfected or P. falciparum-infected) harvested in heparin tubes. After mixing and incubation for 5 min. at room temperature (RT), cells, including Plasmodium parasites, were pelleted by centrifugation at 3500 rpm for 30 min at RT. The cell pellet was washed with 1×PBS containing 1 mM DTT and 0.1 mM PMSF and resuspended in 2× pellet-volume of nuclear extraction buffer (0.5 M NaCl; 20 mM HEPES, pH 7.9; 20% glycerol; 1 mM DTT and 0.1 mM PMSF). Cells and parasites were disrupted by repeated passage through a gauge-G25 syringe. Nuclear content was extracted from the disrupted cells and parasites by rotating for 1 hr. at 4° C. and cell debris spun down at 14.000 rpm for 10 min. at 4° C. The supernatant was collected and used for REEAD.

Enzyme Mediated Circularization of Oligonucleotide Sensors:

Circularization reactions were carried out in 30 μL reaction volumes containing a divalent cation depletion buffer (1 mM Tris-HCl, pH 7.5; 5 mM EDTA) supplemented with 100 nM oligonucleotide sensor(s) as stated in the text. Reactions were initiated by the addition of the purified enzymes (hTopI or pfTopI) and/or cell extracts as described in the text. Incubation was carried out for 30 min at 37° C. before heat inactivating the enzyme(s) for 5 min at 95° C. Samples were exonuclease digested by supplementing the reactions with 7 units exonuclease I (Fermentas) and 70 units exonuclease III (Fermentas) and incubating for 60 min at 37° C., followed by inactivation for 15 min at 80° C.

REEAD—Microscopic Readout:

The 5′-amine-conjugated primer was coupled to CodeLink Activated Slides (SurModics) according to the manufacturer's description. 5 ul circularization reaction sample (supplemented with 100 nM control-circle when stated in the text) was hybridized to the immobilized primers by inbubation for 60 min. at RT (22-25° C.). RCA and microscopic visualization were performed as previously described^{1,7}. Quantification of pfTopI specific signals was performed as previously described¹.

REEAD—HRP Readout:

Primer coupling to NHS-activated M-PVA Ak11 magnetic beads (Chemagen) was performed according to the manufacturer's description. Briefly, 100 μM amine-conjugated primer was incubated with 1.5 pg magnetic beads in 1× coupling buffer (0.05 M HEPES, pH 7.8) for 12 hrs at 4° C. The coupling reaction was quenched by the addition of quenching solution (0.05 M Tris and 0.1% ethanolamine, pH 8.0) followed by incubation for 1 hr at RT. For HRP-mediated detection of pfTopI specific circles, 10 ul circularization reaction sample was hybridized to 15 ng primer-coupled magnetic beads in hybridization buffer (Phi29 polymerase buffer (Fermentas) supplemented with 200 mM NaCl) for 1 hr at RT (22-25° C.). RCA mixture (2 μL of biotin-dNTP mix (mixture of 0.25 mM biotin—dATP and 0.75 mM dATP and 1 mM of other dNTPs), 2 μL of Phi29 buffer (10×) and 2 μL of Phi29 polymerase (Fermentas)) was added to the beads and RCA was carried out at 30° C. for 30 min. followed by further incubation at 37° C. for 3 hrs. Urea unfolding of the RCPs, RCP coupling of avidin-HRP (Sigma-Aldrich) and colorimetric detection (TMB substrate was from Neogen) were performed as previously described¹⁶.

REEAD in Unprocessed Blood Samples in Microfluidic System.

The microfluidic setup consists of two devices: a flow-focusing droplet generator and a drop-trap. Both devices were fabricated by conventional soft lithography techniques²⁵, casting and curing the PDMS prepolymer on a SU-8 3025 (MicroChem) master of a channel height at around 25 μm. PDMS prepolymer (Sylgard 184) was prepared in a 10:1 (base:curing agent) ratio and cured at 65° C. for 1 hr. Prior to the experiments, the channel was wetted with oil/surfactant (EA Surfactant, RainDance) for at least 15 min. Two syringe pumps (Harvard Apparatus) were used to control the flow rates of oil/surfactant and reagents independently, forming monodisperse water-in-oil droplets at a frequency of 0.8-1.5 kHz. The droplet volume and generation frequency was controlled by the flow rate ratio, determined by the competition between continuous phase (carrier fluid (FC-40 fluorocarbon oil (3M): the oil/surfactant, flow rate 22.5 μL/min) and disperse phase (aqueous reagents: blood, lysis buffer and sensors, flow rate 2.5 μL/min). Blood, lysis buffer (10 mM Tris-HCL pH 7.5, 0.5 mM EDTA, 1 mM DTT, 1 mM PMSF, 0.2% Tween 20), and sensors (final sensor concentration in the droplets: S(hTopI): 67 mM, S1: 167 mM, control-circle: 33 mM) were loaded in each their channel in the microfluidic device and droplet generation initiated. The generated droplets were harvested in eppendorf tubes and placed on a primer-printed glass slide prepared as described above. The PDMS drop-trap was gently placed on top of the glass slide. The geometry of the drop-trap was designed according to the size of generated droplets. The droplets were left to exsiccate for 16 hours. Wash, RCA, and hybridization of probes were performed as previously described by Nallur, G. et al. Nucleic Acids Res 29, E118 (2001).

Sequences
Mycobacterium tuberculosis topoisomerase I gene:
SEQ ID NO: 1
TTGGCTGACCCGAAAACGAAGGGCCGTGGCAGCGGCGGCAATGGCAGCGGCCG
GCGACTGGTCATCGTCGAGTCGCCCACCAAGGCGCGCAAGCTGGCCTCCTACCT
GGGCTCTGGCTACATCGTCGAGTCCTCCCGGGGGCACATCCGTGACTTGCCGCG
GGCCGCGTCGGATGTACCCGCAAAGTACAAGTCGCAGCCGTGGGCGCGGCTCG
GGGTCAACGTCGACGCCGACTTCGAACCGCTCTACATCATCAGCCCGGAGAAAC
GGAGCACCGTCAGCGAGCTCAGGGGCCTGCTCAAAGACGTGGACGAGCTGTATC
TGGCCACGGATGGGGACCGTGAGGGCGAAGCTATTGCCTGGCATCTGCTGGAAA
CCCTCAAACCGCGCATACCGGTAAAGCGGATGGTCTTCCACGAGATCACCGAAC
CGGCGATCCGCGCCGCCGCCGAGCACCCCCGCGACCTAGACATCGACCTGGTC
GACGCGCAGGAGACCCGGCGCATCCTGGACCGGCTGTACGGCTACGAAGTCAG
CCCAGTGCTGTGGAAGAAGGTCGCCCCCAAGTTGTCGGCGGGCCGGGTGCAGT
CGGTGGCCACCCGCATCATCGTGGCGCGCGAACGCGACCGCATGGCGTTCCGC
AGCGCGGCCTACTGGGACATCCTTGCCAAGCTGGATGCCAGCGTGTCCGACCCG
GACGCCGCGCCGCCCACCTTCAGCGCCCGGCTGACGGCCGTGGCTGGCCGGCG
GGTGGCCACTGGCCGCGATTTCGACTCGCTGGGCACGCTGCGCAAAGGCGACG
AAGTCATTGTGCTCGACGAGGGGAGCGCGACCGCGTTGGCCGCGGGCCTGGAT
GGCACGCAGCTGACCGTGGCCTCGGCCGAGGAGAAGCCCTACGCCCGGCGCCC
GTACCCGCCGTTCATGACCTCCACGCTGCAGCAAGAGGCCAGCCGCAAGCTGCG
GTTCTCCGCCGAGCGGACGATGAGCATCGCCCAGCGGCTGTACGAAAACGGCTA
CATCACCTATATGCGTACCGACTCCACCACGCTGTCGGAGTCGGCGATCAACGCC
GCACGTACCCAGGCGCGCCAGCTCTACGGCGACGAGTACGTCGCGCCGGCGCC
GCGCCAATACACCCGCAAGGTGAAGAACGCCCAGGAAGCGCACGAGGCTATCCG
GCCCGCCGGTGAAACGTTTGCCACCCCGGACGCGGTGCGTCGCGAACTCGACG
GTCCCAACATTGATGATTTCCGGCTCTATGAGCTGATTTGGCAACGCACCGTAGC
CTCGCAGATGGCCGATGCGCGGGGCATGACGCTGAGCCTGCGGATCACTGGCAT
GTCGGGGCACCAGGAGGTGGTGTTCTCCGCGACCGGACGCACCTTGACGTTCCC
GGGCTTCCTCAAGGCCTACGTGGAGACCGTGGACGAGCTGGTCGGCGGCGAGG
CTGACGATGCCGAGCGGCGACTGCCCCATCTGACCCCGGGTCAACGGTTGGACA
TCGTCGAGTTGACCCCAGACGGCCATGCCACCAACCCGCCGGCCCGCTACACCG
AGGCGTCGCTGGTCAAAGCGCTCGAGGAGCTGGGCATCGGCCGCCCGTCGACC
TACTCGTCGATCATCAAGACCATCCAGGATCGCGGCTACGTGCACAAGAAGGGCA
GTGCACTGGTGCCGTCATGGGTGGCGTTCGCGGTAACCGGTCTGCTCGAGCAGC
ATTTCGGTCGGCTCGTCGACTACGACTTCACCGCGGCGATGGAAGACGAGCTCG
ACGAGATCGCCGCCGGCAACGAGCGCCGCACCAACTGGCTCAACAACTTCTACT
TTGGTGGCGATCACGGTGTGCCCGATTCGGTAGCCCGATCGGGTGGCCTCAAGA
AGCTTGTCGGGATCAATCTCGAGGGCATCGACGCACGAGAAGTAAACTCTATCAA
GCTTTTTGACGACACCCACGGACGCCCCATATATGTTCGGGTGGGCAAGAACGGT
CCCTACCTGGAACGTTTGGTGGCCGGCGACACCGGTGAGCCCACGCCGCAGCG
GGCCAACCTCAGCGACTCGATTACCCCGGACGAGCTGACTCTACAGGTGGCCGA
AGAGCTCTTTGCCACACCGCAACAGGGACGGACTTTGGGCTTGGACCCAGAAAC
CGGCCACGAGATCGTGGCCAGGGAAGGCCGGTTTGGGCCGTATGTGACCGAGA
TCCTGCCGGAGCCTGCGGCTGATGCGGCCGCGGCCGCTCAGGGAGTCAAGAAA
CGCCAGAAGGCCGCCGGGCCCAAACCGCGCACCGGTTCGTTGCTGCGGAGCAT
GGACCTACAGACGGTCACCCTCGAAGACGCGCTGAGGCTGCTGTCACTGCCGCG
CGTGGTCGGAGTGGACCCCGCCTCGGGTGAGGAGATCACCGCGCAGAACGGGC
GCTACGGACCGTATCTAAAGCGCGGCAACGATTCTCGATCACTGGTCACCGAAGA
CCAGATATTCACCATCACGCTCGACGAAGCCCTGAAGATCTACGCAGAGCCGAAA
CGTCGTGGCCGGCAAAGCGCTTCGGCTCCGCCGCTGCGCGAGCTGGGAACAGA
TCCGGCGTCGGGCAAGCCAATGGTCATCAAGGACGGCCGATTCGGGCCGTACGT
CACCGACGGTGAGACCAATGCCAGCCTGCGTAAGGGCGACGACGTGGCTTCCAT
AACCGACGAGCGCGCCGCCGAGCTGTTGGCCGATCGCCGAGCCCGGGGTCCGG
CAAAACGGCCAGCCAGGAAAGCTGCCCGGAAGGTGCCGGCGAAGAAGGCAGCC
AAGCGCGACTAG.
Mycobacterium tuberculosis topoisomerase I protein:
SEQ ID NO: 2
MADPKTKGRGSGGNGSGRRLVIVESPTKARKLASYLGSGYIVESSRGHIRDLPRAAS
DVPAKYKSQPWARLGVNVDADFEPLYIISPEKRSTVSELRGLLKDVDELYLATDGDRE
GEAIAWHLLETLKPRIPVKRMVFHEITEPAIRAAAEHPRDLDIDLVDAQETRRILDRLYG
YEVSPVLWKKVAPKLSAGRVQSVATRIIVARERDRMAFRSAAYWDILAKLDASVSDPD
AAPPTFSARLTAVAGRRVATGRDFDSLGTLRKGDEVIVLDEGSATALAAGLDGTQLTV
ASAEEKPYARRPYPPFMTSTLQQEASRKLRFSAERTMSIAQRLYENGYITYMRTDSTT
LSESAINAARTQARQLYGDEYVAPAPRQYTRKVKNAQEAHEAIRPAGETFATPDAVR
RELDGPNIDDFRLYELIWQRTVASQMADARGMTLSLRITGMSGHQEWFSATGRTLT
FPGFLKAYVETVDELVGGEADDAERRLPHLTPGQRLDIVELTPDGHATNPPARYTEAS
LVKALEELGIGRPSTYSSIIKTIQDRGYVHKKGSALVPSWVAFAVTGLLEQHFGRLVDY
DFTAAMEDELDEIAAGNERRTNWLNNFYFGGDHGVPDSVARSGGLKKLVGINLEGID
AREVNSIKLFDDTHGRPIYVRVGKNGPYLERLVAGDTGEPTPQRANLSDSITPDELTL
QVAEELFATPQQGRTLGLDPETGHEIVAREGRFGPYVTEILPEPAADAAAAAQGVKKR
QKAAGPKPRTGSLLRSMDLQTVTLEDALRLLSLPRVVGVDPASGEEITAQNGRYGPY
LKRGNDSRSLVTEDQIFTITLDEALKIYAEPKRRGRQSASAPPLRELGTDPASGKPMVI
KDGRFGPYVTDGETNASLRKGDDVASITDERAAELLADRRARGPAKRPARKAARKVP
AKKAAKRD.
P.F. Topl
For information, cf:
http://www.ncbi.nlm.nih.gov/gene/812833
Plasmodium falciparum Gene sequence (ACCESSION NC_004326):
http://www.ncbi.nlm.nih.gov/nuccore/NC_004326?report=denbank&from=
445981&to=448500&strand=true
SEQ ID NO: 3

| | |
|---|---|
| 1 | atgcaatcaa tggaaataaa tgataataac agtatcaaga atgaaagtac atctgatgat |
| 61 | gatatattaa ttaataaaat taaacaaaac ttgggtaata ataaatcatg taattctaga |
| 121 | tcttccaaaa aggaatctat aaaaaagcaa aagagcaatt ctgaacttgg tataaaaaag |
| 181 | aacacaaaga aatcattagg tataaaaaaa gaggaagaaa aaaaaaaaca aataagcaaa |
| 241 | agaaaaagta atgaactaaa agaaaaaaat aatttgaaag agggaaaaaa gaaatatgtg |
| 301 | gaaaaaaaat ctagaacagt aaaagatgaa accaagttaa cgaatgttat aaaaaaagaa |
| 361 | actcaaaata ataagaaacc taaaaaatta cttaaaaaat cagaagaaaa ttttgaacca |
| 421 | ataaatagat ggtgggaaaa aatagatgat caaacagata tacaatggaa ttatttagaa |
| 481 | catcgaggat taatattttc ccctccatac gttcaacatc atgtaccaat tttttataaa |
| 541 | agtataaaaa ttgaattaaa tgcaaaatca gaagaattag ctacctattg gtgtagtgca |
| 601 | attggtagtg attattgtac aaaagaaaag tttatattaa atttttttaa aacatttata |
| 661 | aatagtttag aaaatgataa tattataaaa caagagaatg aaacgaaatt aaaaaaagga |
| 721 | gatatatcta attttaagtt tattgatttt atgccaatca aagatcattt attaaaatta |
| 781 | agagaagaaa agttaaataa aacaaaagaa gaaaaagaag aggaaaaaaa aatgagaatg |
| 841 | gaaaaagaat taccatatac atatgcgtta gttgattgga ttcgtgaaaa gatatcaagt |
| 901 | aataaagcag aaccacctgg gttatttaga ggaagaggag aacatccaaa acaaggttta |
| 961 | ttaaaaaaaa gaatttttcc agaagatgtt gtaattaata ttagtaaaga tgcacctgta |
| 1021 | ccacgattat atgataatat gtgtggacat aattggggtg atatatatca tgataataaa |
| 1081 | gtaacatggt tagcttatta taaagatagt ataaatgatc aaataaaata tactttttta |
| 1141 | tctgctcaat caaaatttaa aggatataaa gatcttatga aatatgaaaa tgctcgaaaa |
| 1201 | ttaaaatcat gtgttcataa aattagggaa gattataaaa ataaaatgaa aaataaaaat |
| 1261 | attattgata aacaattagg aacagctgtt tatttaatag attttctagc attaagagta |
| 1321 | ggaggagaaa aagatatcga tgaagaagca gatactgtag gttgttgtag tttaagagta |
| 1381 | gaacatatta gttttgcaca cgatatacct tttaaaagtg tagattcaaa agaacaaaaa |
| 1441 | acaaatgatg aaaaagtaaa taaaatacca ttaccaacaa atttagaaag tatttcatca |
| 1501 | gaagattgtt atataacttt agatttttta ggaaaagata gtatacgata ttttaataca |
| 1561 | gtcaaaatag ataaacaagc atatattaat ataataatat tttgtaaaaa taaaaataga |
| 1621 | gatgaaggag tttttgatca aataacttgt tcaaaattaa atgaatatct aaaagaaatt |
| 1681 | atgcctactt tatcagctaa agtgtttcgt acatataatg cttcaattac attagatcaa |
| 1741 | caattaaaaa gaataaaaga agtttatgga aaaacaacat attcattata ttctggtgaa |
| 1801 | acagaattac acaaatcgaa aaaaagaaaa tctagccatt taacttcaga tacaaatata |
| 1861 | ttaagtgatg caagtgattc tactattaat gatgtaaata acgagtatga tgaaaatgga |
| 1921 | ataaataaaa aactatcata tgctactact gtaggaaaag aaaatgatgt cgatgataaa |
| 1981 | aactcaccaa tagaagttga cgtttcaaat ataaatgaac ttattaattt ttacaataat |
| 2041 | gcaaatagag aagtagccat attatgtaac catcaaagaa gtattccaaa acaacatgat |
| 2101 | acaactatgt caaaaataaa aaaacaaatt gaattatata atgaagatat aaaagaatat |
| 2161 | aaaaaatatt tgcaacattt aaaaaaaaat agtgataaaa aatttatctt tgtttcgaaa |
| 2221 | gtttctactt tagatggaac tttaagacca aataaagtca aagaaaatat gaaagaagaa |
| 2281 | tcttgtaaaa aaaaactaat tactcttata aaaaaagttg aattattaaa taaccaaatg |
| 2341 | aaagtaagag atgataataa aactattgct ttaggtacat ctaaaattaa ttatatggat |
| 2401 | ccaagaataa ctgttgcttt ttgtaaaaaa tttgaaatac ccatagaaaa agtatttaat |
| 2461 | agaagtttaa gacttaaatt tccttgggcc atgtttgcta caaaaaattt tacattttaa. |

//
Plasmodium falciparum Protein sequence (ACCESSION XP_001351663):
http://www.ncbi.nlm.nih.gov/protein/XP_001351663.1
SEQ ID NO: 4

| | |
|---|---|
| 1 | mqsmeindnn siknestsdd dilinkikqn lgnnkscnsr sskkesikkq ksnselgikk |
| 61 | ntkkslgikk eeekkkqisk rksnelkekn nlkegkkkyv ekksrtvkde tkltnvikke |
| 121 | tqnnkkpkkl lkkseenfep inrwwekidd qtdiqwnyle hrglifsppy vqhhvpifyk |
| 181 | sikielnaks eelatywcsa igsdyctkek filnffktfi nslendniik qenetklkkg |
| 241 | disnfkfidf mpikdhllkl reeklnktke ekeeekkmrm ekelpytyal vdwirekiss |
| 301 | nkaeppglfr grgehpkqgl lkkrifpedv viniskdapv prlydnmcgh nwgdiyhdnk |
| 361 | vtwlayykds indqikytfl saqskfkgyk dlmkyenark lkscvhkire dyknkmknkn |
| 421 | iidkqlgtav ylidflalry ggekdideea dtvgccslry ehisfandip fksvdskeqk |
| 481 | tndekvnkip lptnlesiss edcyitldfl gkdsiryfnt vkidkqayin iiifcknknr |
| 541 | degvfdqitc sklneylkei mptlsakvfr tynasitldq qlkrikevyg kttyslysge |
| 601 | telhkskkrk sshltsdtni lsdasdstin dvnneydeng inkklsyatt vgkendvddk |
| 661 | nspievdvsn inelinfynn anrevailcn hqrsipkqhd ttmskikkqi elynedikey |
| 721 | kkylqhlkkn sdkkfifvsk vstldgtlrp nkvkenmkee sckkklitli kkvellnnqm |
| 781 | kvrddnktia lgtskinymd pritvafckk feipiekvfn rslrlkfpwa mfatknftf. |

Substrates:
M.T. Topl
Mycobacterium tuberculosis substrate
TbSub-ID33
SEQ ID NO: 5
5′p-CAGAGTGCGCAGTTGG-CCTCAATGCACATGTTTGGCTCC-
GAGCGAGCTTCCGCT-tgacatcccaata-3′.
Mycobacterium tuberculosis substrate
TbSub-ID33
SEQ ID NO: 6
5′p-CAGAGTGCGCAGTTGG-tctct-CCTCAATGCACATGTTTGGCTCC-tctct-
GAGCGAGCTTCCGCT-tgacatcccaata-3′.
Mycobacterium tuberculosis topoisomerase I target
SEQ ID NO: 7
CGCTtg.
P.F. Topl
Plasmodium falciparum substrate
Tp-Id33/PfTop1(S1)
SEQ ID NO: 8
5′-TCTAGAAAGTATAGGAACTTCGAACGACTCAGAATG-ACTGTGAAGATCGCTTAT-
CCTCAATGCACATGTTTGGCTC-CATTCTGAGTCGTTCGAAGTTCCTATACTTT-3′.
Plasmodium falciparum substrate
sub Tp-IdS3/PfTop1(S2)
SEQ ID NO: 9
5′-CATACATTATACGAAGTTATGAGCGTCTGAGTATG-ACTGTGAAGATCGCTTAT-
CAGTGAATGCGAGTCCgTCTACT-CATACTCAGACGCTCATAACTTCGTATAATGT-3′.
Plasmodium falciparum substrate
PF-subs-Topl primer, may have 3′-amine, ID16
SEQ ID NO: 10
5′-ATTTTTAA-ACTGTGAAGATCGCTTAT-TTAAAAATTTTTCTAAGTCTTTTTTCC-
CCTCAATGCTGCTGCTGTACTAC-GAAAAAAGACTTAGAAAAAT-3′.
Plasmodium falciparum substrate
PF-subs-ver1-Topl/PfTop1(S3)
SEQ ID NO: 11
5′-ATTATAATTTTTTGGAACTTCGAACGACTCAGAATG-ACTGTGAAGATCGCTTAT-
CCTCAATGCACATGTTTGGCTCC-CATTCTGAGTCGTTCGAAGTTCCAAAAAATT-3′.
Plasmodium falciparum substrate
PF-subs-ver2-Topl/PfTop1(S4)
SEQ ID NO: 12
5′-TTATAATTTTTTGGAACTTCGAACGACTCAGAATG-ACTGTGAAGATCGCTTAT-
CCTCAATGCATGTTTGGCTCC-CATTCTGAGTCGTTCGAAGTTCCAAAAAATT-3′.
Plasmodium falciparum substrate
PF-subs-ver5-Topl
SEQ ID NO: 13
5′-TTTATAAAGTATAGGAACTTCGAACGACTCAGAATG-ACTGTGAAGATCGCTTAT-
CCTCAATGCACATGTTTGGCTCC-CATTCTGAGTCGTTCGAAGTTCCTATACTTT.
Plasmodium falciparum substrate
PF-subs-ver3-Flp ID33
SEQ ID NO: 14
5′-AAATTTTTTTTGGAACTTCGAACGACTCAGAATG-AGGCTCAATCTAATGGAC-
CCTCAATGCACATGTTTGGCTCC-CATTCTGAGTCGTTCGAAGTTCCAAAAAA-3′.
Plasmodium falciparum substrate
PF-subs-ver4-Flp ID33
SEQ ID NO: 15
5′-TTTATAAAGTATAGGAACTTCGAACGACTCAGAATG-AGGCTCAATCTAATGGAC-
CCTCAATGCACATGTTTTTTTTGCTCC-CATTCTGAGTCGTTCGAAGTTCCTATACTTT.
Plasmodium falciparum substrate
SEQ ID NO: 16
5′TCTAGTAAGTATAGGAACTTCGAACGACTCAGAATGACTGTGAAGATCGCTTATCCTCAATG
CACATGTTTGGCTCCCATTCTGAGTCGTTCGAAGTTCCTATACTTA.
Plasmodium falciparum substrate
PfTop1(S5)
SEQ ID NO: 17
5′ATTTTTCTAAGTCTTTTAGATCGAACGACTCAGAATGACTGTGAAGATCGCTTATCCTCAAT
GCACATGTTTGGCTCCCATTCTGAGTCGTTCGATCTAAAAGACTTAGA-3′.
Plasmodium falciparum topoisomerase I target
SEQ ID NO: 18
TCTAGTAAG-(N)ₓ-CTTA,
where N is A, T, C, or G, and x is between 5 and 500.
Plasmodium falciparum topoisomerase I target
SEQ ID NO: 19
ATTTTTCTA-(N)ₓ-TAGA
where N is A, T, C, or G, and x is between 5 and 500.
Probe sequence
SEQ ID NO: 20
CCTCAATGCACATGTTTGGCTCC.
Probe sequence
SEQ ID NO: 21
CAGTGAATGCGAGTCCgTCTACT.
Probe sequence
SEQ ID NO: 22
CCTCAATGCTGCTGCTGTACTAC.
Primer binding sequence
SEQ ID NO: 23
ACTGTGAAGATCGCTTAT.
Primer binding sequence
SEQ ID NO: 24
AGGCTCAATCTAATGGAC.
Substrate for human Topoisomerase I, S(Topl).
S(Topl):
SEQ ID NO: 25
5′-AGAAAAATTT TTAAAAAAAC TGTGAAGATC GCTTATTTTT
TTAAAAATTT TTCTAAGTCT TTTAGATCCC TCAATGCTGC TGCTGTACTA
CGATCTAAAA GACTTAGA-AMINE-3′.
Substrate for Flp-recombinase, S(Flp), or pfTop1.
S(Flp):
SEQ ID NO: 26
5′-TCTAGAAAGT ATAGGAACTT CGAACGACTC AGAATGAGGC
TCAATCTAAT GGACCCTCAA TGCACATGTT TGGCTCCCAT TCTGAGTCGT
TCGAAGTTCC TATACTTT-3′.
RCA-primers,
matching S(Topl),
SEQ ID NO: 27
5′-AMINE-CCAACCAACC AACCAAATAA GCGATCTTCA CAGT-3′.;
matching S(Flp) or pfTop1,
SEQ ID NO: 28
5′-AMINE-CCAACCAACC AACCAAGTCC ATTAGATTGA GCCT-3′.;
matching S(Cre),
SEQ ID NO: 29
5′-AMINE-CCAACCAACC AACCAACATA GAGTCCTGGT GAGC-3′.;
detection probes,
p(Topl),
SEQ ID NO: 30
5′-“F”-GTAGTACAGC AGCAGCATTG AGG-3′.;
p(Flp),
SEQ ID NO: 31
5′-“F”-GGAGCCAAAC ATGTGCATTG AGG-3′.;
p(Cre),
SEQ ID NO: 32
5′-“F”- AGACGGACTC GCATTCACTG-3'..
“F” indicates fluorescent labeling, which was Cy5, rhodamine, or FITC

1-55. (canceled)

56. A method of detecting an enzyme in a sample or identifying a microorganism expressing the enzyme in the sample, the method comprising:
a) providing the sample,
b) providing a nucleic acid substrate targeted by the enzyme,
c) loading the sample of step a) and the nucleic acid substrate of step b) into a sample chamber comprising a flow through channel, wherein droplets comprising the sample and the nucleic acid substrate are generated,
d) transfer the droplets from the sample chamber to a droplet retaining means through the flow through channel,
e) capturing one or more single droplets in individual cavities of the droplet retaining means, wherein each single droplet is spatially isolated from other droplets, and
f) detecting, in one or more captured droplets, nucleic acid substrate processed by the enzyme, wherein the presence of processed nucleic acid substrate is indicative of the presence of the enzyme, the microorganism, or both.

57. The method according to claim 56, wherein the enzyme is a DNA-modifying enzyme selected from the group consisting of: nucleases, ligases, recombinases, topoisomerases and helicases.

58. The method according to claim 57, wherein the enzymes is type I topoisomerase.

59. The method according to claim 56, wherein the sample chamber comprises one or more inlet channels, one or more outlet channels for the generated drops, or both.

60. The method according to claim 59, wherein the sample chamber comprises four inlet channels for the individual loading of sample, nucleic acid substrate, cell lysis buffer and oil, respectively.

61. The method according to claim 59, wherein the one or more flow through channels, inlet channels and/or outlet channels have a diameter of 10-50 micrometers, such as approximately 25 micrometers.

62. The method according to claim 56, wherein at least 80% of the droplets comprise one or no cells and/or the droplets have a volume of 500 pL or less, such as between 50 and 200 pL.

63. The method according to claim 56, wherein between approximately 4 and 30% of the droplets comprises one cell, and approximately 0.1 to 10% of the droplets comprise two or more cells.

64. The method according to claim 56, wherein the droplet retaining means is a porous solid support comprising cavities for capturing droplets of 50 pL to 100 microlitres.

65. The method according to claim 56, wherein the enzyme is a type I topoisomerase and the processed nucleic acid substrate cleaved and/or ligated by the type I topoisomerase.

66. The method according to claim 56, wherein the ligation is intramolecular ligation of the 3′-terminus of the nucleic acid substrate to the 5′-terminus of the nucleic acid substrate, thereby generating a circular nucleic acid product.

67. The method according to claim 56, wherein the captured single droplets are exsiccated after being captured.

68. The method according to claim 56, wherein the processed nucleic acid substrate is detected by southern blotting, polymerase chain reaction, RT-PCR, qPCR, RFLD, primer extension, DNA array technology, a linear amplification technique, isothermal amplification and/or rolling circle amplification.

69. The method according to claim 56, wherein the processed nucleic acid substrate is detected by rolling circle amplification.

70. The method of claim 69, wherein the nucleic acid rolling circle amplification is performed by
a) providing to the one or more captured droplets at least one oligonucleotide primer, which is capable of hybridizing to a circularized nucleic acid substrate,
b) hybridizing the at least one oligonucleotide primer to the circularized nucleic acid substrate,
c) providing a nucleic acid polymerase and nucleotides,
d) generating a rolling circle amplification product by extending the at least one oligonucleotide primer using the circularized nucleic acid substrate as template, and
e) detecting the rolling circle amplification product.

71. The method according to claim 70, wherein the at least one oligonucleotide primer is selected from SEQ ID NO: 23-24.

72. The method according to claim 70, wherein the oligonucleotide primer and/or nucleotides is immobilized on a solid support.

73. The method according to claim 70, wherein the oligonucleotide primer and/or nucleotides is immobilized on the droplet retaining means.

74. The method according to claim 70, wherein one or more of the nucleotides comprise one or more detectable labels.

75. The method according to claim 74, wherein the rolling circle amplification product is detected via its incorporation of the nucleotides comprising one or more detectable labels.

76. The method according to claim 74, wherein the rolling circle amplification product is detected by hybridization of a labelled nucleic acid probe to multiple sites of the rolling circle amplification product.

77. The method according to claim 76, wherein the nucleic acid probe is labelled with one or more fluorescent dyes, radioactive nucleotides and/or biotinylated nucleotides.

78. The method according to claim 77, wherein the nucleic acid probe is coupled to an enzyme, wherein the enzyme is capable of converting a substrate into a detectable product.

79. The method according to claim 78, wherein the enzyme is fused with streptavidin and coupled to the nucleic acid probe via interaction with the biotinylated nucleotides incorporated in the nucleic acid probe, and wherein the enzyme is horse-radish peroxidase.

80. The method according to claim 56, wherein the microorganism is Plasmodium falciparum, Mycobacterium tuberculosis, or Mycobacterium bovis.

81. The method according to claim 56, wherein the sample originates from a human being or a bovine subject.

82. The method according to claim 56, wherein the sample is depleted of divalent cations.

83. The method according to claim 56, wherein the nucleic acid substrate is substantially targeted by a type I topoisomerase of the microorganism and at least partially by any type I topoisomerase native to the sample, wherein the type I topoisomerase native to the sample is a human type I topoisomerase or bovine type I topoisomerase.

84. The method according to claim 56, wherein the nucleic acid substrate is at least partly double-stranded, wherein the nucleic acid substrate is provided as a single nucleic acid that folds into a secondary hairpin structure comprising a double-stranded target region.

85. The method according to claim 56, wherein the nucleic acid substrate comprises a sequence selected from any one of SEQ ID NO: 5-24, a sequence at least 90% identical thereto, or a part of at least 5 consecutive nucleotides of any of the sequences.

86. The method according to claim 56, wherein the microorganism is selected from the Plasmodium Genus, and the nucleic acid substrate comprises a sequence selected from any one of SEQ ID NO: 8-19, a sequence at least 90% identical thereto, or a part of at least 5 consecutive nucleotides of any of the sequences.

87. The method according to claim 86, wherein the nucleic acid substrate comprises the sequence TCTAGTAAG-(N)_{X}-CTTA or ATTTTTCTA-(N)_{X}-TAGA, where N is A, T, C, or G, and x is between 5 and 500 (SEQ ID NOs: 18 or 19).

88. The method according to claim 56, wherein the microorganism is selected from the Plasmodium Genus, and the nucleic acid substrate comprises a sequence, with at least 80% identity to any one of SEQ ID NOs: 8-17, and which comprise the sequence TCTAGTAAG-(N)_{X}—CTTA or ATTTTTCTA-(N)_{X}-TAGA, where N is A, T, C, or G, and x is between 5 and 500 (SEQ ID NOs: 18 or 19)

89. The method according to claim 56, wherein the microorganism is selected from the Mycobacterium Genus, and the nucleic acid substrate comprises a sequence selected from any one of SEQ ID NO: 5-7, a sequence at least 90% identical thereto, or a part of at least 5 consecutive nucleotides of any of the sequences.

90. The method according to claim 89, wherein the nucleic acid substrate comprises SEQ ID NO: 7.

91. The method according to claim 56, wherein the microorganism is selected from the Plasmodium Genus, and the nucleic acid substrate comprises a sequence, with at least 80% identity to any one of SEQ ID NO: 5 and 6, and which comprises SEQ ID NO: 7.

92. A method of detecting a disease in a subject, the method comprising identifying a microorganism in a sample from the subject using the method of claim 56, wherein detecting the microorganism in the sample is indicative of the disease being present in the subject.

93. The method according to claim 92, wherein the disease is malaria and the microorganism is selected from the Plasmodium genus; or the disease is human tuberculosis, bovine tuberculosis, or both and the microorganism is selected from the Mycobacterium genus.
